SEQUENCE LISTING (9 sequences)

SEQ ID NO: 1 - RNA, 21 nt, synthetic construct
  source 1..21: mol_type = other RNA
  modified_base 2: mod_base = um
  modified_base 4: mod_base = um
  modified_base 5: mod_base = cm
  modified_base 7: mod_base = um
  modified_base 10: mod_base = cm
  modified_base 11: mod_base = cm
  modified_base 14: mod_base = cm
  modified_base 20: mod_base = OTHER; note = deoxythymidine
  modified_base 18: mod_base = cm
  modified_base 21: mod_base = OTHER; note = deoxythymidine; note = phosphorothioated DNA base
atatcatggccgacaagcatt

SEQ ID NO: 2 - RNA, 21 nt, synthetic construct
  source 1..21: mol_type = other RNA
  modified_base 12: mod_base = cm
  modified_base 17: mod_base = um
  modified_base 20: mod_base = OTHER; note = deoxythymidine
  modified_base 21: mod_base = OTHER; note = deoxythymidine; note = phosphorothioated DNA base
tgcttgtcggccatgatattt

SEQ ID NO: 3 - RNA, 21 nt, synthetic construct
  source 1..21: mol_type = other RNA
  modified_base 2: mod_base = um
  modified_base 4: mod_base = um
  modified_base 6: mod_base = um
  modified_base 7: mod_base = um
  modified_base 10: mod_base = cm
  modified_base 11: mod_base = cm
  modified_base 12: mod_base = um
  modified_base 14: mod_base = um
  modified_base 16: mod_base = um
  modified_base 17: mod_base = um
  modified_base 21: mod_base = OTHER; note = deoxythymidine; note = phosphorothioated DNA base
  modified_base 20: mod_base = OTHER; note = deoxythymidine
atgtattggcctgtattagtt

SEQ ID NO: 4 - RNA, 21 nt, synthetic construct
  source 1..21: mol_type = other RNA
  modified_base 2: mod_base = um
  modified_base 5: mod_base = um
  modified_base 7: mod_base = cm
  modified_base 12: mod_base = cm
  modified_base 15: mod_base = um
  modified_base 17: mod_base = cm
  modified_base 20: mod_base = OTHER; note = deoxythymidine
  modified_base 21: mod_base = OTHER; note = deoxythymidine; note = phosphorothioated DNA base
ctaatacaggccaatacattt

SEQ ID NO: 5 - RNA, 100 nt, synthetic construct
  source 1..100: mol_type = other RNA
  modified_base 1: mod_base = gm; note = phosphorothioated RNA base
  modified_base 2: mod_base = OTHER; note = phosphorothioated 2'-O-methyladenosine
  modified_base 3: mod_base = cm; note = phosphorothioated RNA base
  modified_base 97: mod_base = um; note = phosphorothioated RNA base
  modified_base 98: mod_base = um; note = phosphorothioated RNA base
  modified_base 99: mod_base = um; note = phosphorothioated RNA base
gaccaggatgggcaccacccgttttagagctagaaatagcaagttaaaataaggctagtccgttatcaacttgaaaaagtggcaccgagtcggtgctttt

SEQ ID NO: 6 - RNA, 100 nt, synthetic construct
  source 1..100: mol_type = other RNA
  modified_base 1: mod_base = OTHER; note = phosphorothioated 2'-O-methyladenosine
  modified_base 2: mod_base = um; note = phosphorothioated RNA base
  modified_base 3: mod_base = um; note = phosphorothioated RNA base
  modified_base 97: mod_base = um; note = phosphorothioated RNA base
  modified_base 98: mod_base = um; note = phosphorothioated RNA base
  modified_base 99: mod_base = um; note = phosphorothioated RNA base
atttcaggtaagccgaggttgttttagagctagaaatagcaagttaaaataaggctagtccgttatcaacttgaaaaagtggcaccgagtcggtgctttt

SEQ ID NO: 7 - RNA, 100 nt, synthetic construct
  source 1..100: mol_type = other RNA
  modified_base 1: mod_base = gm; note = phosphorothioated RNA base
  modified_base 2: mod_base = cm; note = phosphorothioated RNA base
  modified_base 3: mod_base = um; note = phosphorothioated RNA base
  modified_base 97: mod_base = um; note = phosphorothioated RNA base
  modified_base 98: mod_base = um; note = phosphorothioated RNA base
  modified_base 99: mod_base = um; note = phosphorothioated RNA base
gctgaagcactgcacgccgtgttttagagctagaaatagcaagttaaaataaggctagtccgttatcaacttgaaaaagtggcaccgagtcggtgctttt

SEQ ID NO: 8 - DNA, 127 bp, synthetic construct
  source 1..127: mol_type = other DNA
  modified_base 1: mod_base = OTHER; note = phosphorothioated DNA base
  modified_base 2: mod_base = OTHER; note = phosphorothioated DNA base
  modified_base 125: mod_base = OTHER; note = phosphorothioated DNA base
  modified_base 126: mod_base = OTHER; note = phosphorothioated DNA base
gccacctacggcaagctgaccctgaagttcatctgcaccaccggcaagctgcccgtgccctggcccaccctcgtgaccaccctgagccacggcgtgcagtgcttcagccgctaccccgaccacatga

SEQ ID NO: 9 - protein, 12 aa, synthetic construct
YHWYGYTPQNVI

(19) United States
(12) Patent Application Publication
(10) Pub. No.: US 2026/0159854 A1
(43) Pub. Date: Jun. 11, 2026

(54) NOVEL CARRIERS FOR NUCLEIC ACID AND/OR PROTEIN DELIVERY

(71) Applicant: LUDWIG-MAXIMILIANS-UNIVERSITÄT MÜNCHEN, München (DE)

(72) Inventors: Ernst WAGNER, München (DE); Lun PENG, München (DE); Simone BERGER, München (DE); Sophie THALMAYR, München (DE); Paul FOLDA, München (DE); Franziska HAASE, München (DE); Melina GRAU, München (DE); Janin GERMER, München (DE); Mina YAZDI, München (DE); Eric WEIDINGER, München (DE); Tobias BURGHARDT, München (DE); Ricarda DURBEN, München (DE); Johanna SEIDL, München (DE)

(21) Appl. No.: 19/132,459

(22) PCT Filed: Nov. 21, 2023
(86) PCT No.: PCT/EP2023/082604

§ 371 Date: May 23, 2025

(30) Foreign Application Priority Data
Nov. 23, 2022 (EP) 22209198.5

(51) Int. Cl. C12N 15/88; A61K 48/00

(52) U.S. Cl. CPC C12N 15/88; A61K 48/0033

(57) ABSTRACT

The invention relates to a carrier comprising at least one polar cationizable domain (PCD), two or more apolar cationizable domains (ACD) and at least one branching connector (BC), wherein the two or more ACDs are linked by at least one branching connector to at least one PCD, wherein the PCD is an oligo(alkylamino) acid, an ε-poly-L-lysine or an ε-poly-L-lysine-6-Ahx, and the ACD is a lipo amino fatty acid (LAF) comprising a tertiary amine. The invention further relates to nanoparticles comprising said carrier and a cargo, wherein the cargo comprises a nucleic acid and/or a protein and to a pharmaceutical composition comprising said nanoparticles and to its use in therapy or in in vitro culture.

### FIELD OF THE INVENTION

The invention relates to a carrier comprising at least one polar cationizable domain (PCD), two or more apolar cationizable domains (ACD) and at least one branching connector (BC), wherein the two or more ACDs are linked by at least one branching connector to at least one PCD, wherein the PCD is an oligo(alkylamino) acid, an ε-poly-L-lysine or an ε-poly-L-lysine-6-Ahx and the ACD is a lipo amino fatty acid (LAF) comprising a tertiary amine. The invention further relates to nanoparticles comprising said carrier and a cargo, wherein the cargo comprises a nucleic acid and/or a protein and to a pharmaceutical composition comprising said nanoparticles and to its use in therapy or in in vitro culture.

### BACKGROUND

Gene therapy is a potent field of modern, personalized medicine for tumor and genetic diseases, and its impact is growing continuously. There are still numerous challenges with regard to delivery, particularly in the case of synthetic therapeutic nucleic acid carriers. Cellular internalization of nucleic acids across lipid membranes is hindered by size and negative charge. Thus, appropriate carrier systems are required for efficient delivery of nucleic acid, comprising extracellular stabilization and protection, efficient cellular uptake and intracellular cargo release. In addition, these shuttles should be biocompatible and ideally non-toxic. Suitable carrier systems for non-viral gene delivery are nanoparticles, such as lipid nanoparticles (LNPs) and polymer complexes (polyplexes). LNPs have recently become well known by the tremendous success of the messenger RNA (mRNA) vaccines against COVID-19 from Pfizer-BioNTech (BNT162b2, also known as Comirnaty®) and Moderna (mRNA-1273, also known as Spikevax®). Another approved drug using LNPs is Onpattro®, the first small interfering RNA (siRNA) therapeutic approved, which silences the expression of the protein transthyretin in the liver causing transthyretin amyloidosis (ATTR). Polyplexes are formed by electrostatic interaction between cationic polymers and anionic nucleic acids and are also used for nucleic acid delivery, such as plasmid DNA (pDNA), mRNA or siRNA.

Cationic transfection agents comprise cationic lipids as well as cationic polymers and peptides for complexation of negatively charged nucleic acids into lipoplexes or polyplexes, respectively (P. L. Felgner et al., Human gene therapy 1997, 8, 511). The cationic carriers support attachment of positively charged nucleic acid nanoparticles to cells and endocytosis via various intracellular routes. With regard to crossing cellular lipid membranes unto the cytosol, lipoplexes take advantage of fusion of cationic lipids with anionic lipids of the endosome host membrane. In contrast, endosomal escape of polyplexes appears less effective and is mechanistically not completely clarified. Various mechanisms are considered, including host membrane rupture after cationic polymer interactions with negatively charged endosomal membrane components such as bis(monoacylglycero)phosphate (D. J. Brock et al., Cell Chem Biol 2020, 27, 1296). In several cases, inclusion of endosomolytic peptides or hydrophobic domains was found to strongly enhance carrier efficiencies. Cationizable polymers (‘proton sponges’) such as polyethylenimine (O. Boussif et al., Proc. Natl. Acad. Sci. U.S.A 1995, 92, 7297; B. A. Demeneix et al., Artificial Self-Assembling Systems for Gene Delivery, American Chemical Society, Washington, USA 1996; A. Hall et al., Mol Ther 2017, 25, 1476) display favorable transfection properties for several reasons. Endosomal protonation enhances their positive charge density for localized phospholipid membrane disruption, which has been postulated to be additionally promoted by osmotic swelling of endosomes. Importantly, endosomal pH-specific protonation is considered to avoid a direct damage of the cytosolic membrane and thus to reduce cytotoxic side effects. Indeed, the endosomal protonation strategy was also successfully applied in the development of cationizable lipids, lipidoids and corresponding lipid nanoparticle (LNP) formulations (Y. Sato et al., J Controlled Release 2019, 295, 140; K. T. Love et al., Proc Natl Acad Sci USA 2010, 107, 1864; P. R. Cullis and M. J. Hope, Mol Ther 2017, 25, 1467). Nevertheless, even for LNP formulations the endosomal barrier is still significant. For initial LNP versions, a limited escape of only 1-2% of cargo was reported (J. Gilleron et al., Nature biotechnology 2013, 31, 638; A. Wittrup et al., Nature biotechnology 2015, 33, 870).

Previously, the cationizable polyethylenimine motif has been introduced into artificial oligoamino acids. Using solid-phase synthetic technology and these artificial oligoamino acids, small libraries of sequence-defined artificial peptides with precise chemical structure and topology were designed and evaluated for delivery of various nucleic acid or protein cargos (D. Schaffert et al., Org Lett 2011, 13, 1586; D. Schaffert et al., Angewandte Chemie (International ed. in English) 2011, 50, 8986; Y. Wang and E. Wagner, Pharmaceutics 2020, 12; D. J. Lee et al., Biomaterials 2016, 77, 98; R. Order et al., Pharmaceutical research 2017, 34, 161; P. Zhang et al., Advanced Functional Materials 2015, 25, 6627; D. J. Lee et al., Bioconjugate chemistry 2017; P. M. Klein et al., Biomaterials 2018, 178, 630). Apart from cationizable amino acids, tested libraries included also additional hydrophilic or lipophilic domains and residues as well as different topologies based on branching points. Notably, upon such chemical evolution of nanocarrier libraries (F. Freitag and E. Wagner, Adv Drug Deliv Rev 2021, 168, 30) distinct expected differences in cargo requirements were observed (C. Scholz and E. Wagner, J Controlled Release 2012, 161, 554). pDNA polyplexes containing only hydrophilic cationizable domains and optionally polyethyleneglycol (PEG) shielding and receptor targeting domains were found suitable for pDNA compaction, intravenous delivery and tumor-specific therapeutic gene transfer in vivo (P. Kos et al., Nanoscale 2015, 7, 5350; S. Urnauer et al., Mol Ther 2016, 24, 1395). For the far smaller double-stranded siRNA, lipidic residues and additional stabilizing measures were important for formation of stable siRNA lipopolyplexes and efficient in vivo gene silencing (J. Luo et al., J Controlled Release 2021, 329, 919). Also, for Cas9/single guide RNA (sgRNA) ribonucleoprotein (RNP) polyplexes and phosphorodiamidate morpholino oligomers (PMO) conjugates, incorporation of lipidic residues was advantageous (J. Kuhn et al., Advanced Functional Materials 2019, 29, 1906432; J. Kuhn et al., Bioconjugate Chemistry 2020). For larger nucleic acid chains, such as pDNA and mRNA, hydrophobic stabilization presents a double-edged sword; high stability may be useful during the delivery process, but also hamper release at the intracellular target site of action (S. Hama et al., Mol Ther 2006, 13, 786; S. Berger et al., Biomacromolecules 2021, 22, 1282; A. Krhač Levačić et al., J Controlled Release 2021, 339, 27). For example, in a library of T-shaped lipo-oligomers containing saturated fatty acids with chain lengths between C2 and C18, the carriers containing short C6 to C10 fatty acids displayed lower polyplex stability, but a higher endosomolytic activity and a 500-fold higher gene expression than the C18 carrier analogues (S. Berger et al., Biomacromolecules 2021, 22, 1282). For mRNA lipopolyplexes the balancing act between stability and cargo release was even more difficult and was handled best by placing a bioreducible disulfide bond between the cationizable hydrophilic polycationic backbone and the hydrophobic domain for dynamic mRNA release in the cytosol (A. Krhač Levačić et al., J Controlled Release 2021, 339, 27).

Despite these advances, there is still a need for further improved carriers for delivery of various nucleic acid and/or protein cargos into cells in vitro and in vivo.

### SUMMARY OF THE INVENTION

In the carriers of the present invention a dynamic protonation-triggered change in lipophilicity of the lipidic carrier subdomain is provided. In contrast to previous carriers based on polar cationizable backbones modified with standard lipidic residues, established polar cationizable backbones were combined with a new lipidic domain which is cationizable. As demonstrated in the following, this strategy results in novel carriers for pDNA, mRNA, and siRNA transfer with up to several hundred-fold higher efficiency than the previous carrier generation even at very low dosage.

The present invention relates to a carrier comprising at least one polar cationizable domain (PCD), two or more apolar cationizable domains (ACD) and at least one branching connector (BC), wherein the two or more ACDs are linked by at least one branching connector to at least one PCD, wherein
(a) the PCD
   (i) is an oligo(alkylamino) acid of formula I:

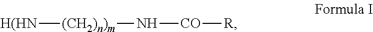

wherein n=2 or 3, alternating 2 and 3 or alternating 3 and 2; and m=3 or 4; and wherein R is
   —(CH₂)_{y}—CO₂H, wherein y=2, 3 or 4;

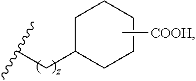

wherein z=0 or 1, such as

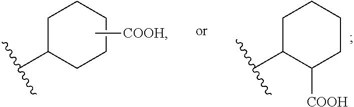

—CH₂-(cyclohexylene)-CH₂—CO₂H; or
—CH₂—C(CH₃)₂—CH₂—CO₂H or CH₂—C(CH₂—CH₃)₂—CH₂—CO₂H
—CH₂—O—CH₂—CO₂H; or
—CH₂—NH—CH₂—CO₂H; or
(ii) is an epsilon-poly-L-lysine comprising structure, preferably an (ε-lysine)ₙ-6-amino hexanoic acid ((^{ε}K)ₙ-6-Ahx), (ε-lysine)ₙ₊₁ ((K)ₙ₊₁) or (ε-lysine)ₙ ((^{ε}K)ₙ), wherein n=2, 3 or 4, preferably 2 or 3;
(b) the BC is a bisamide connector selected from the group consisting of L-lysine, L-ornithine and an artificial amino acid comprising two amino groups and a carboxyl group; and
(c) the ACD is a lipo amino fatty acid (LAF) comprising a tertiary amine of formula II:

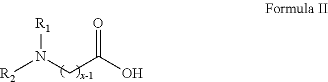

wherein x is 4-12, and R1 and R2 is an acyclic alkane or alkene, wherein R1 is C6-C16 and R2 is C1-C16 and wherein R1 and R2 may be the same or different.

The carrier according to the invention is for nucleic acid and/or protein delivery, preferably for nucleic acid delivery or cargo comprising nucleic acid. The free C-terminal carboxyl group of the PCD in the carrier may further be modified or further coupled. In certain embodiments, the oligo(alkylamino) acid is a tetraethylenepentamine or a triethylenetetramine of formula Ib: H(HN—(CH₂)₂)_{m=3 or 4}—NH—CO—R; preferably a tetraethylenepentamine. The carrier may further comprise (a) a disulfide building block between the at least one PCD and the two or more ACDs, and/or (b) a spacer between the at least one PCD and the two or more ACDs. The carrier according to the invention may further comprise one or more histidine, cysteine and/or arginine.

In certain embodiments, the carrier further comprises a terminal functional group selected from an azido-group and a thiol group, preferably a terminal azido-lysine, azido-hexane, cysteine or homocysteine. The carrier may further comprise a targeting ligand coupled via the azido-group or the thiol group.

The carrier according to the invention preferably has a topology selected from the group consisting of:
(a) a bundle structure

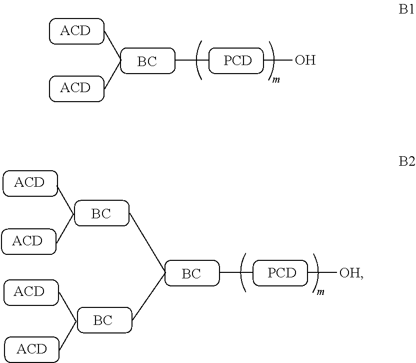

wherein m=1, 2 or 3; and
(b) a U-shape structure

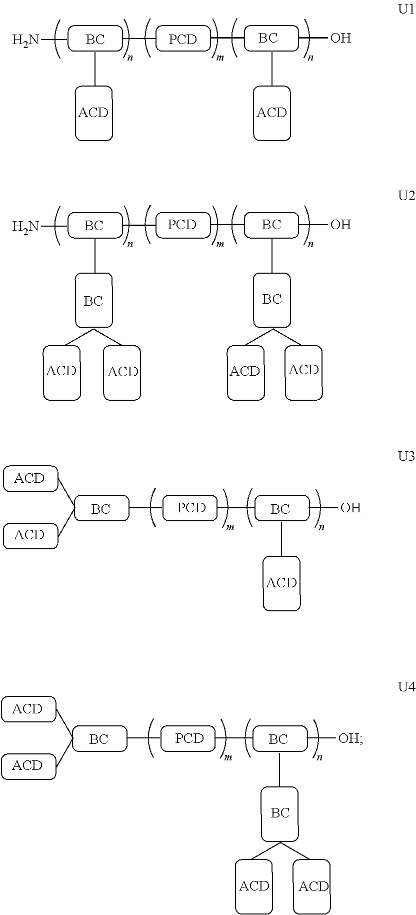

wherein n=1 or 2 and m=1, 2 or 3, preferably 1 or 2.

The PCD to ACD ratio is preferably between 1:2 to 1:4 in the carrier, preferably 1:2 for U-shapes (wherein PCD is 1 or 2) and/or 1:2 to 1:4 for B2 structures. In some embodiments, the carrier is produced by solid phase synthesis.

In another aspect, the invention relates to a nanoparticle comprising the carrier of the invention, further comprising a cargo, wherein the cargo comprises a nucleic acid and/or a protein, preferably a nucleic acid. In certain embodiments the cargo comprises RNA or DNA, preferably the cargo comprises mRNA, Cas mRNA/gRNA, siRNA, microRNA (miRNA), polyinosinic:polycytidylic acid (poly(I:C)), a phosphodiamidate-morpholino-oligomer (PMO), a non-viral DNA expression vector (e.g., pDNA), Cas protein/gRNA ribonucleoprotein (RNP) or mixtures thereof. The nanoparticle of the invention may be a complex (polyplex) formed by the carrier mixed with the cargo, a lipid nanoparticle (LNP) comprising the carrier loaded with the cargo, or a complex formed by the cargo covalently coupled to the carrier.

In yet another aspect, the invention relates to a pharmaceutical composition comprising the nanoparticle of the invention further comprising at least one pharmaceutically acceptable excipient.

In yet another aspect, the invention relates to the nanoparticle according to the invention for use in therapy.

In yet another aspect, the invention relates to the nanoparticle according to the invention for use in treating or preventing cancer, a genetic disease, an infectious disease or an autoimmune disease.

In yet another aspect of the invention, an in vitro method for introducing a nucleic acid and/or a protein into mammalian cells is provided, comprising contacting a mammalian cell in vitro with the nanoparticle according to the invention comprising a nucleic acid and/or a protein as cargo. The nucleic acid may be RNA or DNA. Preferably the cargo is mRNA, Cas mRNA/gRNA, siRNA, miRNA, polyinosinic:polycytidylic acid (poly(I:C)), phosphodiamidate-morpholino-oligomer (PMO), non-viral DNA expression vector (e.g., pDNA), or Cas protein/gRNA ribonucleoprotein (RNP).

In yet another aspect of the invention a use of the carrier according to the invention or the nanoparticle according to the invention for cellular delivery of a nucleic acid and/or a protein, preferably of a nucleic acid, is provided.

### DESCRIPTION OF THE FIGURES

FIG. 1: Library design of novel lipo amino fatty acid (LAF) containing carriers. Building blocks (A) used to synthesize nucleic acid carriers with apolar and polar cationizable domains (B) connected via lysine into different topologies (for combs, m=1, 2 or 4 and n=2, 4, 6; for bundles m=1 or 2; for U-shapes m=1 or 2 and n=1 or 2) (C); and chemical formula of B2 and U1 topologies with Stp and m=1 or 2 and n=1 or 2 (D). 6-Ahx, 6-aminohexanoic acid; LAF, lipo amino fatty acid; (L)-K, lysine; Stp, succinyl-tetraethylenepentamine. Nomenclature of LAFs: The number (8, 10, 12, 14, 16) expresses the length of the terminal alkyl chains; the two letters represent the used amino fatty acid (“Oc”, 8-aminooctanoic acid; “He”, 6-aminohexanoic acid; “Bu”, 4-aminobutanoic acid).

FIG. 2: Luciferase expression in N2a cells at 24 h after transfection. LAF 12Oc containing carriers formulated as pDNA polyplexes at N/P 12 and tested at a dose of 200 ng pCMVLuc/well in comparison to positive control LPEI at N/P 6. A) LAF carriers with comb and T-shape topologies; B) LAF carriers with bundle and U-shape topologies. C) Dose titration of pDNA polyplexes formed with different 12Oc carriers at N/P 12 in comparison to positive control LPEI N/P 6. Evaluation of 200, 100, 50, 26, and 14 ng pCMVLuc/well. The ratio specified for the different topologies, such as in U1-2:4 indicates the PCD:ACD content.

FIG. 3: Influence of LAF variations on transfection results in N2a cells. Polyplexes formed with LAF analogs of different topologies (A, B2-1:4; B, U1-1:2; C, U1-2:4) at indicated N/P ratios (B2-1:4, N/P 18; U1-1:2, N/P 18; U1-2:4, N/P 12) were tested on N2a cells. Luciferase expression of N2a cells at 24 h after transfection with pDNA polyplexes at a dose of 200 ng pCMVLuc/well (n=3; mean±SD). As positive control LPEI N/P 6 was used. The ratio specified for the different topologies, such as in U1-2:4, indicates the PCD:ACD content.

FIG. 4: Dose titration of mRNA polyplexes formed with different 12Oc containing carriers. Luciferase expression in N2a cells at 24 h after transfection with mRNA polyplexes formed at indicated N/P ratios in comparison to positive controls succPEI (w/w 4) and 1218 (N/P 12). Evaluation of 63, 31, and 16 ng mRNA/well (CleanCap Fluc mRNA (5moU), Trilink). Positive controls were also transfected at a high dose of 250 ng mRNA/well. Luciferase expression evaluated after 1:100-dilution and shown as RLU values after background subtraction (HBG-treated control cells) (n=3, mean±SD). The ratio specified for the different topologies, such as in U1-2:4 indicates the PCD:ACD content.

FIG. 5: Luciferase expression in N2a cells at 24 h after transfection. Comparison of the LAF containing carrier 1722 with its analog 1725 containing the control motif DodOc (amide) instead of the LAF (tertiary amine). Carriers were tested in N2a cells with A) both carriers formulated as pDNA polyplexes at an N/P ratio of 12 and tested at a dose of 200 ng pCMVLuc/well, and B) both carriers formulated as mRNA polyplexes at N/P 12 at a dose of 62.5 ng CleanCap® FLuc mRNA (5moU)/well. Luciferase expression in mRNA-treated cells evaluated after 1:100-dilution of cell lysate in PBS and shown as RLU values after background subtraction (HBG-treated control cells).

FIG. 6: Luciferase expression in N2a and DU145 cells. Comparison of different polar domains (Stp vs ^{ε}K-^{ε}K-6-Ahx) in LAF containing carriers of the same topology (U1). mRNA polyplexes formulated at indicated N/P ratio with CleanCap® FLuc mRNA (5moU) and tested at a dose of 62.5 ng mRNA/well in A) N2a cells; B) DU145 cells. Luciferase expression in mRNA-treated cells evaluated after 1:10-dilution (A, N2a) or 1:100-dilution (B, DU145) of cell lysates in PBS and shown as RLU values after background subtraction (HBG-treated control cells).

FIG. 7: Influence of LAF variations on transfection results. Polyplexes formed with LAF analogs of different topologies (A, B2-1:4; B, U1-1:2; C, U1-2:4) at indicated N/P ratios (B2-1:4, N/P 18; U1-1:2, N/P 18; U1-2:4, N/P 12) were tested on N2a cells (A-C) and DU145 cells (D). Luciferase expression of N2a cells at 24 h after transfection with mRNA polyplexes at a dose of 31 ng CleanCap® FLuc mRNA (5moU)/well as well as of DU145 cells at 24 h after transfection with mRNA polyplexes at a dose of 63 ng CleanCap® FLuc mRNA (5moU)/well (n=3; mean±SD). The positive control succPEI (w/w 4) was transfected at a dose of 250 ng mRNA/well. Luciferase expression in mRNA-treated cells evaluated after 1:100-dilution of cell lysate in PBS and shown as RLU values after background subtraction (HBG-treated control cells). The ratio specified for the different topologies, such as in U1-2:4 indicates the PCD:ACD content.

FIG. 8: In vivo experiment in N2a tumor-bearing A/J mice. Ex vivo luciferase expression assay of indicated organs after intravenous injection of 150 μL of mRNA polyplexes (CleanCap® Fluc mRNA (5moU) Trilink, San Diego, CA, USA). A) Comparison of the transfection efficiency of polyplexes with 10 μg mRNA/animal at different time-points (i.e., 6 h and 24 h after injection). SuccPEI formulated at w/w 2 and 1611 formulated at N/P 18. B) Transfection efficiency of U1-2:4 carriers 24 h after injection. Formulation with 10 μg mRNA/animal at N/P 12. C) Injection of indicated doses of mRNA polyplexes, read-out at 6 h after injection (succPEI w/w 2, 1611 N/P 18, 1752 N/P 18). Experiments were carried out in groups with n=5 animals and results are shown as mean±SD. n.d.=not detectable. The ratio specified for the different topologies, such as in U1-2:4 indicates the PCD:ACD content.

FIG. 9: Co-delivery efficiency of mRNA/pDNA polyplexes. Polyplexes formed with different LAF carriers at N/P 18 containing both mCherry mRNA and pEGFP-N1 pDNA at fixed weight ratio of 1:1, were evaluated at indicated (25 or 50 ng) combined nucleic acid doses per well in N2a cells. Successful co-delivery of both nucleic acid cargos shown by combined mCherry and GFP expression in the same cell as determined by flow cytometry.

FIG. 10: Particle size (Z-average), polydispersity index (PDI), and zeta potential of siRNA polyplexes. Different LAF containing carriers and siCtrl (25 μg/mL) were formulated at N/P 18 followed by A) size and PDI, and B) zeta potential measurement via DLS & ELS (n=3, mean±SD).

FIG. 11: Gene silencing activity of siRNA polyplexes in N2a/eGFPLuc cells. siRNA polyplexes (25 μg/mL siRNA) were formulated with A) different LAF containing carriers at N/P 18 and tested at doses of 62.5, 31.2, and 15.6 ng siRNA/well in comparison to the dose titration of B) positive controls of succPEI (w/w 4) and 1214 (N/P 12). As siRNAs, eGFP-targeted siRNA (siGFP) and control siRNA (siCtrl) were used. Luciferase expression was measured 48 h after transfection without change of the medium. RLU was presented as percentage of the luciferase gene expression of the HBG buffer-treated cells (n=3, mean±SD).

FIG. 12: Gene silencing activity of siRNA polyplexes (N/P 18) in three different cell lines (N2a/eGFPLuc, DU145/eGFPLuc, and KB/eGFPLuc). siRNA polyplexes were formulated with different LAF containing carriers (N/P 18) and tested at dose of 15.6 ng siRNA/well in comparison to positive controls succPEI (w/w 4) and 1214 (N/P 12) polyplexes both at a concentration of 500 ng siRNA/well. As siRNAs, eGFP-targeted siRNA (siGFP) and control siRNA (siCtrl) were used. Luciferase expression was measured 48 h after transfection without change of the medium. RLU was presented as percentage of the luciferase gene expression of the HBG buffer-treated cells (n=3, mean±SD).

FIG. 13: Luciferase gene expression of mRNA LNPs. N2a (A), HepG2 (B) and Huh7 (C) cells were transfected with mRNA LNPs at 62.5 ng CleanCap® FLuc mRNA (5moU) per well. LNPs were prepared at N/P 9 and different molar ratios and compared to MC3 and SM-102 LNPs at N/P 4.5 as positive controls (LNP compositions see Table A in methods section “mRNA and siRNA lipid nanoparticle (LNP) formulations”). Luciferase gene expression in the cell lysates (after 1:10-dilution in PBS in the case of N2a and HepG2 cells, no dilution in the case of Huh7 cells) was measured at 24 h post transfection and is shown as RLU values after background subtraction (HBG-treated control cells).

FIG. 14: Ex vivo luciferase gene expression of mRNA LNPs in different organs. N2a tumor-bearing A/J mice were intravenously injected with LNP formulations containing 3 or 10 μg CleanCap® FLuc mRNA (5moU) in 150 μL HBG per mouse. LNPs were prepared at N/P 9 in different molar ratios with and without post-functionalization with human transferrin (hTf) (LNP compositions see Table A+B in methods section “mRNA and siRNA lipid nanoparticle (LNP) formulations”). Luciferase gene expression per gram (g) organ at 20 h post injection (n=3; mean±SD).

FIG. 15: Gene silencing activity of siRNA LNPs in three different cell lines (N2a/eGFPLuc, KB/eGFPLuc, and CT26/eGFPLuc). siRNA LNPs were prepared with novel LAF carriers at N/P 9 and tested at doses of 63 and 31 ng siRNA/well (LNP compositions see Table A in methods section “mRNA and siRNA lipid nanoparticle (LNP) formulations”). MC3 and SM-102 LNPs (N/P 4.5) were included as positive controls at similar concentrations. As siRNAs, eGFP-targeted siRNA (siGFP) and control siRNA (siCtrl) were used. Luciferase expression was measured 48 h after transfection without change of the medium. RLU was presented as percentage of the luciferase gene expression of the HBG buffer-treated cells (mean±SD; n=3).

FIG. 16: Efficiency of Cas9 mRNA/sgGFP1 polyplex carriers determined by flow cytometry. Dose titration of Cas9 mRNA/sgRNA polyplexes formed with best performing carriers containing LAFs 12Oc or 8Oc at indicated N/P ratios. eGFP knock out efficiency in dependency of the total amount of RNA (Cas9 mRNA and sgGFP1 at weight ratio 1:1), evaluated by the percentage of eGFP negative cells at 72 h after treatment of N2a/eGFPLuc cells.

FIG. 17: Efficiency of Cas9 mRNA/sgGFP1 polyplex carriers determined by flow cytometry. Dose titration of Cas9 mRNA/sgRNA polyplexes formed with carriers containing indicated LAFs at N/P ratio 18 and 24. eGFP knock out efficiency in dependency of the total amount of RNA (Cas9 mRNA and sgGFP1 at weight ratio 1:1), evaluated by the percentage of GFP negative cells at 72 h after treatment of N2a/eGFPLuc cells.

FIG. 18: Efficiency of Cas9 mRNA/sgRNA polyplex carriers determined by flow cytometry. Transfection efficiency of Cas9 mRNA/sgRNA polyplexes formed with LAF containing carriers at indicated N/P ratios in HeLa mCherry-DMDex23-eGFP cells in dependency of the total amount of RNA (Cas9 mRNA and sgDMDex23 at weight ratio 1:1), determined by the percentage of cells expressing mCherry protein 3 d post treatment. The lowest RNA doses of 5 and 2.5 ng per well were only tested in the case of 1621 polyplexes.

FIG. 19: Efficiency of Cas9 mRNA/sgRNA polyplex carriers after incubation in 90% serum by flow cytometry. Polyplexes formed with carrier 1621 at different N/P ratios were pre-incubated for 2 h in 90% FBS and transfected on HeLa mCherry-DMDex23-eGFP cells at different doses of total RNA (Cas9 mRNA and sgDMDex23 at weight ratio 1:1). Transfection efficiency was evaluated by the percentage of cells expressing mCherry protein 3 d post treatment.

FIG. 20: HDR-mediated GFP to BFP conversion and NHEJ-mediated GFP knock out efficiency of Cas9 mRNA/sgRNA/ssDNA polyplexes. Polyplexes formed with carrier 1611 at N/P 18 containing Cas9 mRNA and sgRNA at fixed ratio of 1:1 were evaluated in HeLa GFPd2 cells. A) Dose titration of total transfected nucleic acid from 150 ng to 25 ng per well at fixed weight ratios of total RNA to the ssDNA template 1:0.5. B) Testing of different weight ratios of total RNA to ssDNA (ranging from 1:0 to 1:10), at a fixed dose of 100 ng total nucleic acid per well.

FIG. 21: Comparison of RNP and Cas9 mRNA/sgRNA lipopolyplex carriers at N/P ratio 24. Gene editing efficiency evaluated on HeLa mCherry-DMDex23-eGFP by the percentage of cells expressing mCherry protein 3 days post treatment determined by flow cytometry. (A) Transfection efficiency of 2.5 nM, 5 nM and 10 nM RNP (Cas9 Protein and sgRNA at a molar ratio of 1:1) containing carriers formulated with LAF structures. This is equivalent to 8 ng, 16 ng and 32 ng sgRNA per well. (B) Transfection efficiency of 2.5 nM, 5 nM and 10 nM sgRNA (Cas9 mRNA and sgDMDex23 weight ratio 1:1) containing carriers formulated with LAF structures being equivalent to 8 ng, 16 ng and 32 ng sgRNA per well.

FIG. 22: Carriers replacing Stp by different polar building blocks (Stp analogs) in PCD. (A) Structures of novel Stp analogs dmGtp and Stt, and topologies of novel carriers. (B-D) Polyplexes formed with carriers containing different Stp analogs at indicated N/P ratios (B, C, 12Oc-U1-1:2 (N/P 18); D, 12Oc-U1-2:4 (N/P 12) and 12Bu-B2-1:4 (N/P 18)) were tested in N2a cells (B, D) and DC2.4 cells (C). Luciferase expression at 24 h after mRNA transfection. Luciferase expression in mRNA-treated cells evaluated after 1:100-dilution (N2a cells) or 1:10-dilution (DC2.4 cells) of cell lysate in PBS and shown as RLU values after background subtraction (HBG-treated control cells) (n=3; mean+SD) for indicated doses of mRNA/well (CleanCap FLuc mRNA (5moU), Trilink). Positive control succPEI (w/w 4) was also transfected at a higher mRNA dose of 250 ng/well.

FIG. 23: Novel carriers connecting Stp and LAF via ornithine as branching connector (BC). (A) Chemical structures of ornithine carriers 1813, 1814, 1827. (B-C) Comparison of polyplexes either formed with ornithine connector carriers or corresponding lysine connector analogs (B, C, 12Oc-U1-1:2, 8Oc-B2-1:4, C, 12Bu-B2-1:4). (B) Transfection efficacy of pDNA formed at N/P 18 at a pCMVLuc dose of 50 or 15 ng per well in Huh7 cells. Luciferase gene expression was determined at 24 h after transfection (n=3; mean+SD). (C) Transfection efficacy of mRNA polyplexes formed at N/P 18 at a CleanCap FLuc mRNA (5moU) dose of 50 or 15 ng per well in Huh7 cells. Luciferase expression in Huh7 cells at 24 h after transfection (n=3; mean+/−SD), evaluated after 1:100-dilution and shown as RLU values after background subtraction (HBG-treated control cells).

FIG. 24: Bioreducible carriers containing disulfide building blocks. (A) Chemical structures of carriers. (B-E) pDNA transfections. (B) Transfection efficacy of pDNA polyplexes formed with either 1611 or disulfide containing analog 1821 (both 12Oc-U1-1:2) at N/P 18 in comparison to LPEI (N/P 6) at a pCMVLuc dose of 200 ng/well in HeLa cells. Luciferase gene expression was determined at 24 h after transfection (n=3; mean±SD). (C) Relative metabolic activity of HeLa cells (5000 cells/well) after treatment with pDNA polyplexes at a DNA dose of 200 ng/well, determined via CellTiter-Glo assay at 24 h after transfection. Data presented in relation to HBG buffer-treated control cells. (D) Transfection efficacy of pDNA polyplexes of 1730 and its disulfide containing analog 1824 (both 8Oc-B2-2:4) formed at N/P 18 in comparison to LPEI (N/P 6) at a pCMVLuc dose of 200 ng/well in HeLa cells. Luciferase gene expression was determined at 24 h after transfection (n=3; mean±SD). (E) Relative metabolic activity of HeLa cells (5000 cells/well) after treatment with pDNA polyplexes at a DNA dose of 200 ng/well for 24 h. Data presented in relation to HBG buffer-treated control cells. (F-G) mRNA transfections. mRNA polyplexes formed at N/P 24 with 1621 or its bioreducible ssbb containing analog 1792 (both 8Oc-B2-1:4) were tested on HeLa cells (5000 cells/well) at indicated doses of CleanCap FLuc mRNA (5moU) per well. (F) Luciferase expression in HeLa cells at 24 h after transfection (n=3; mean±SD), evaluated after 1:100-dilution and shown as RLU values after background subtraction (HBG-treated control cells). (G) Relative metabolic activity of HeLa cells at 24 h after transfection, determined via CellTiter-Glo Assay in relation to HBG-treated control cells (n=3; mean±SD). (H-1) Annexin/PI assay on HeLa cells (40,000 cells/well) at 24 h after treatment with (H) pDNA polyplexes formed with either 1611 or disulfide analog 1821 (both 12Oc-U1-1:2), or 1730 and its disulfide analog 1824 (both 8Oc-B2-2:4) at N/P 18 (125 ng/well) in comparison to HBG-buffer treated cells; or (1) mRNA polyplexes formed with either 1621 or disulfide-analog 1792 (both 8Oc-B2-1:4) at N/P 18 (78 ng/well) in comparison to HBG-buffer treated cells.

FIG. 25: Novel two-arm carrier 1851 containing εK as polar cationizable domain (PCD) instead of Stp. (A) Chemical structure of carrier 1851. (B) Luciferase expression in N2a cells at 24 h after mRNA transfection (n=3; mean+SD) with Stp-free 12Oc symmetric 2-arm carrier 1851 at indicated N/P ratios and at doses of 50 and 15 ng CleanCap FLuc mRNA (5moU) per well, in comparison to positive control Stp2 containing carrier 1717. Evaluation after 1:100-dilution and results shown as RLU values after background subtraction (HBG-treated control cells).

FIG. 26: Carriers applied for delivery of cytotoxic poly(inosine:cytosine), poly(I:C). Anti-tumoral activity of poly(I:C) polyplexes against KB (A), U87 (B), and HCT116 cells (5000 cells/well) (C) were evaluated upon a 48-h treatment via an MTT assay. Poly(I:C) polyplexes were formulated with different carriers of 1611 (N/P18), 1719 (N/P12), and 1752 (N/P24) in HBG (12.5 μg/μL poly(I:C)) and tested at different doses (100, 50, 25, and 10 ng) of poly(I:C) in a total volume of 100 μL medium per well. For each dose, the same doses of poly(I) polyplexes were applied as negative control. (D) LPEI (N/P 6) and Lipofectamine 2000 in KB cells were used as positive controls.

FIG. 27: EGF receptor-targeted PEG-shielded pDNA polyplexes. (A) Click synthesis of lipidic anchor DSPE-PEG70-GE11. (B) Formation of pDNA polyplexes. (C) Luciferase expression of Huh7 cells at 24 h after pDNA polyplextransfection (n=3; mean+SD). Unmodified, shielded (25% PEG), and targeted (12.5% PEG; 12.5% PEG70-GE11) polyplexes were transfected in comparison to HBG negative and LPEI positive control at N/P 12 and at indicated pCMVLuc pDNA doses.

FIG. 28: In vivo expression of luciferase mRNA nanoparticles after intramuscular injection of polyplexes or 1621 LNPs. Intramuscular application of 3 μg CleanCap FLuc (5moU) mRNA polyplexes or LNPs in 50 μL HBG into the musculus biceps femoris of 6 week old BALB/c mice (n=4; mean+SD). Luciferase expression was evaluated at 6 h post treatment via an ex vivo luciferase assay of the injected muscle. Polyplexes of LAF carriers 1611, 1752, and 1762 were all formed at an N/P ratio of 24. For LNPs, N/P ratio of the LAF carrier 1621 and SM-102 was 24 and 6, respectively. Molar ratios of lipidic components in applied LNP formulations were 38.5:10:1.5:50 mol % (Chol:DSPC:PEG-DMG:SM-102) and 47.6:23.8:4.8:23.8 mol % (Chol:DOPE:PEG-DMG:1621) for SM-102 and 1621 LNPs, respectively.

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. Similarly, the term “encoding” or “encodes” is intended to be open-ended and allows the presence or addition or one or more other features, elements or components. Furthermore, singular and plural forms are not used in a limiting way. As used herein, the singular forms “a”, “an” and “the” designate both the singular and the plural, unless expressly stated to designate the singular only.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

A “peptide bond” as used herein is an amide type of covalent chemical bond linking two amino acids via the carboxylic group of one amino acid with the amino group of the other amino acid. Typically, the peptide bond refers to the bond between two α-functional groups of two amino acids between C-1 of one amino acid (carboxylate) and the α-amino group the other amino acid, which may also be referred to as α-peptide bond in contrast to an amide bond, e.g., with the ε-amino group of lysine (side chain amino group) or the δ-amino group of ornithine (side chain amino group of the non-proteinogenic amino acid). For the artificial amino acids described herein the peptide bond is analogously formed e.g., between a C-terminal carboxylic group of one artificial amino acid and the N-terminal primary amino group of the adjacent amino acid (natural or artificial).

The term “nucleic acid sequence” is used interchangeably with “polynucleotide” and refers to DNA or RNA of any length. The DNA may be a vector, particularly a non-viral DNA expression vector, or a linear double stranded or single stranded DNA molecule. A non-viral DNA expression vector includes without being limited thereto, a plasmid (pDNA), a plasmid-derived minicircle DNA, a dumbbell-shaped DNA (dbDNA, doggybone DNA), a cosmid, a bacterial artificial chromosome (BAC), a yeast artificial chromosome (YAC) or a mammalian artificial chromosome (MAC). The DNA may be as well a linear double stranded or single stranded DNA molecule, including without being limited thereto, a donor DNA, or a coding DNA sequence with or without expression regulating elements, such as a promoter and a termination site. The person skilled in the art would understand that although the non-viral DNA expression vector is typically circular, it may also be linearized, i.e., packaged or complexed following linearization. Also, antisense oligonucleotides (ASO) may be single strands of DNA or RNA. The term “pDNA” as used herein refers to a plasmid DNA. The term “RNA” as used herein refers to ribonucleic acid. RNA includes without being limited thereto messenger RNA (mRNA), as well as transfer RNA (tRNA), ribosomal RNA (rRNA), small nuclear RNA (snRNA) and other non-coding RNA, such as micro RNA (miRNA), small interfering RNA (siRNA), polyinosinic:polycytidylic acid (poly(I:C)), piwi-interacting RNA (piRNA), small nucleolar ribonucleic acid (snoRNA), long-non-coding RNA (LncRNA), small hairpin RNA (shRNA) or guide RNA (gRNA).

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as human or rodent cells, including without being limited thereto Chinese hamster ovary (CHO) cells, Neuro-2a cells, BHK cells, HEK293 cells, HeLa cells, HepG2 cells or derivatives thereof as well as primary cells, particularly human primary cells. Mammalian cells as used herein refer to all cells of mammalian origin, such as human or rodent cells.

The term “about” as used herein refers to a variation of 10% of the value specified, for example, about 50% carries a variation from 45 to 55%.

The term “sequence-defined” as used herein refers to a sequential assembly of the compounds, particularly using a solid-phase supported synthesis, such as common Fmoc solid-phase synthesis (SPS). In the present context this involves the use of artificial amino acids with appropriate protecting groups, such as compatible with Fmoc SPS together with Fmoc α-amino acids.

The term “guide RNA” abbreviated to gRNA as used herein refers to an RNA that is partially complementary to a target DNA locus and guides the Cas protein endonuclease to this site. The gRNA may be a CRISPR RNA (crRNA), a crRNA that pairs with trans-activating crRNAs (tracrRNA), an artificial single-guide RNA, an artificial prime editing guide RNA (pegRNA) or other RNA molecules which form a complex with a Cas protein and guide it to the target DNA sequence. The term “single-guide RNA” abbreviated to sgRNA as used herein refers to an artificial RNA consisting of tracr RNA, crRNA and an artificial RNA linker. It also includes modified/improved sgRNAs, such as tru-gRNA, using a spacer sequence with <20 nucleotides complementary to the protospacer target, and hp-sgRNA, comprising an extension on the 5′end of the spacer. Wherein modified/improved means compared to the canonical guide RNA. Many Cas12 nucleases are guided by a single crRNA. The term “artificial” in the context of an RNA means an engineered non-naturally occurring RNA. A guide RNA may also be chemically modified to increase stability, reduce TLR activation and increase specificity.

The binding of the gRNA to the Cas protein results in the formation of a ribonucleoprotein (RNP) complex. The CRISPR/Cas system composed of the gRNA and a Cas protein as a targeted nuclease can identify a targeting sequence next to a protospacer adjacent motif (PAM) through guidance by a gRNA which is specific for the targeting sequence and then cleave the DNA (or RNA in specific cases) at specific sites. The gRNA therefore confers sequence specificity to the RNP complex and several gRNA (with different target specificity) can be used with the CRISPR/Cas system. In a preferred embodiment the gRNA is a sgRNA. The RNP complex may be delivered as Cas mRNA/gRNA or Cas protein/gRNA RNP.

The terms “Cas” and “Cas protein” are used interchangeably herein and refers to a CRISPR-associated endonuclease. Suitable Cas proteins include, without being limited thereto, type II Cas proteins, e.g., Cas9 (such as SpCas9, SaCas9, CjCas9, StCas9 or NmeCas9); type V Cas proteins, e.g., Cas12a (formerly Cpf1), Cas12f (formerly Cas14), Cas12b (formerly c2c1), Cas12i, Cas12e (formerly CasX) or Cas12g; and type VI Cas proteins, e.g., Cas13a, all of which include engineered variants thereof (engineered Cas variants). Engineered Cas variants include, without being limited thereto, variants with altered PAM compatibilities, such as less restrictive or different PAM compatibility of Cas9 or Cas12 variants (e.g., Anzalone et al., Nature Biotechnology, 38, 2020: pages 824-844, supplementary Table 1); variants with higher DNA specificity, such as variants with reduced off-target Cas nuclease activity (e.g., eSpCas(1.1), SpCas9-HF1, HypaCas9, evoCas9, Sniper-Cas9, HiFiCas9, enAsCas12a-HF1); base editors (e.g., as listed in Anzalone et al., Nature Biotechnology, 38, 2020: pages 824-844, supplementary Tables 2 and 3); CRISPR-associated transposases and engineered Cas-domain-fused transposase and recombinase systems; and prime editors, as well as mutated or truncated variants, such as Cas nickase and dCas. For example, a Cas nickase (e.g., nickase Cas9n and Cas9D10A) comprising an inactivating mutation in one or more of the nuclease domains (cleaving only one of the DNA strands) and a nuclease-deficient dCas mutant with only sgRNA binding ability, optionally further fused to another enzyme, expanded the conventional editing applications. A Cas protein ortholog refers to one of two or more homologous Cas proteins derived from different species, for example Cas9 orthologs include, without being limited thereto, Cas9 protein derived from a different bacterial species, such as SpCas9 derived from Streptococcus pyogenes, SaCas9 derived from Staphylococcus aureus, CjCas9 derived from Campylobacter jejuni, StCas9 derived from Streptococcus thermophilus, and NmeCas9 from Neisseria meningitidis. Cas orthologs typically differ in the recognized PAM sequences and size. The most often used Cas9 protein is SpCas9.

The term “engineered” in the context of a protein, particularly a Cas protein, means an artificial, non-naturally protein, particularly Cas protein, such as a protein with a deleted domain and/or a fusion protein and/or a mutated protein, wherein the mutation may for example result in a different specificity, e.g., a different PAM specificity, or an inactivated or enhanced enzyme activity of the protein or of one or more of the distinct nuclease domain(s) (e.g., RuvC and/or HNH of Cas9).

The term “Cas9 protein” as used herein refers to Cas9 nucleases that are guided by guide RNAs to generate predominantly blunt-end DSBs using two distinct nucleases (RuvC and HNH), as well as engineered variants thereof, e.g., Cas9 nickase comprising an inactivated HNH and/or RuvC nuclease domain and the nuclease-deficient dCas9.

Typically, a double-strand break at the target site in the cellular genome is introduced. Strand breaks can be repaired by non-homologous end joining (NHEJ), which can introduce insertions or deletions (indels) or in the presence of a donor DNA by homology-directed repair (HDR). The donor DNA may be double-stranded DNA (dsDNA) or single-stranded DNA (ssDNA), such as single stranded oligonucleotide donors (ssODNs). It can be delivered as plasmid, linear double-stranded DNA or single stranded DNA. Also, the donor DNA may be co-delivered together with the RNP complex or may be delivered separately by non-viral or viral delivery. For example, the donor DNA may be delivered using a separate nanoparticle, wherein the carrier may be the same, i.e., the carrier according to the invention or the carrier may be different. Exemplary viral-delivery methods include, e.g., adeno-associated virus (AAV), lentivirus or adenovirus, preferably AAV.

The term “indel” or “indels” as used herein refers to a variety of insertions and deletions, typically introduced by error-prone non-homologous end joining processes during the cellular repair of double-stranded DNA breaks (DSBs). Indel products that result from DSB cannot be controlled, but are not random. In open reading frames they usually generate frameshift mutations in coding sequences that abrogate protein function.

The term “chimeric single-guide RNA” abbreviated to cgRNA as used herein refers to a modified sgRNA which carries a first sequence to generate double-stranded breaks and a second sequence for homology-directed repair.

The term “base editor” as used herein introduces targeted point mutations without the requirement of DSBs or donor DNA template. Typically, a base editor containing a catalytically impaired CRISPR-Cas nuclease (that cannot make DSBs), such as Cas9 nickases or dCas9, fused to a DNA deaminase enzyme. Two main classes of base editors have been developed, cytosine base editors (CBEs), which catalyze the conversion from C/G base pairs to T/A base pairs (cytidine deaminase); and adenine base editors (ABEs), which catalyze conversion from A/T base pairs to G/C base pairs (adenosine deaminase). The base editor may optionally further be fused to proteins that modify the DNA repair machinery, (e.g., uracil glycosylase inhibitor domain (UGI) for CBEs or N-methylpurine DNA glycosylase for ABEs. Most base editors, such as CBEs BE3, BE4, BE4max and ABEs ABE7.10, ABEmax use a Cas nickase. The person skilled in the art understands that the Cas nickase nicks the non-deaminated DNA strand. Alternatively, a base editor may comprise a dCas mutant.

The term “prime editor” as used herein refers to a combination of a Cas9 nickase domain (inactivated HNH nuclease) and an engineered reverse transcriptase domain, which may be fused or untethered. Prime editors can introduce all possible types of point mutations, including all base pair conversions, small insertions and small deletions in a precise and targeted manner with favorable editing to indel ratios. The prime editor is targeted to the editing site by an engineered prime editing guide RNA (pegRNA), which specifies the target site in its spacer sequence and the desired edit in an extension that is typically at the 3′end of the pegRNA. Upon targeted binding, the Cas9 RuvC nuclease domain nicks the PAM-containing DNA strand and uses the newly liberated 3′ end at the target DNA site to prime reverse transcription using the extension of the pegRNA. Successful priming requires that the extension in the pegRNA contain a primer binding sequence (PBS) that hybridizes with the 3′end of the nicked target DNA strand to form a primer-template complex. The reverse transcriptase domain then copies the template from the pegRNA extension into the genomic DNA directly adding the edited sequence to the target locus. The edited 3′flap replaces the redundant 5′flap, presumably by cellular DNA repair processes. Finally, the non-edited complementary strand is replaced by DNA repair using the edited strand as a template. This is facilitated by the addition of a sgRNA to nick the non-edited strand, which stimulates resynthesis of the non-edited strand using the edited strand as a template, resulting in a double-stranded edited DNA. Known prime editors, without being limited thereto are PE1 (fusion of Cas9 nickase to wild-type Moloney murine leukemia virus (M-MLV) reverse transcriptase (RT), PE2 (fusion of Cas9 nickase to engineered pentamutant M-MLV RT with increased editing efficiency), PE3 (PE2 and pegRNA and additional sgRNA), PE3b (PE3 using a nicking sgRNA that targets only the edited sequence), PE4 (PE2 in combination with DNA mismatch repair inhibiting protein MLH1dn), PE5 (PE3 in combination with MLH1dn) and PEmax (optimization of PE2) or split Prime editors, such as Split-PE (Cas9 nickase and reverse transcriptase are expressed separately and fused at the mRNA or protein level). The use of prime editors is known in the art, such as derivable from Anzalone et al., (Nature Biotechnology, 38, 2020: pages 824-844), Chen et al., (Cell, 184(22), 2021: pages 5635-5652.e29), Liu, B. et al., (Nat Biotechnol 40, 2022: 1388-1393) and Grunewald, J. et al., (Nat Biotechnol (2022); doi: 10.1038/s41587-022-01473-1).

The term “phosphodiamidate-morpholino-oligomer (PMO)” as used herein refers to an oligomer molecule of a nucleic acid analog used to modify gene expression, particularly to knock down gene function. It contains DNA bases attached to a backbone of methylenemorpholine rings linked through phosphorodiamidate groups. PMOs block access of other molecules to small (˜25 base) specific sequences of the base-pairing surface of ribonucleic acid (RNA).

The term “polyinosinic:polycytidylic acid (poly(I:C))” as used herein refers to mismatched double-stranded RNA with one strand being a polymer of inosinic acid, the other of a polymer of cytidylic acid. It is structurally similar to double-stranded RNA and mimics double stranded viral DNA. It is used, typically in the form of its sodium salt, as an immunostimulant that simulates viral infections and is known to interact with toll-like receptor 3 (TLR3).

The term “click chemistry” as used herein refers to a class of highly specific, in many cases biorthogonal, covalent conjugation reactions, that are modular, efficient, relatively insensitive to solvent parameters, water and oxygen. Typical click reactions, without being limited thereto are copper-catalyzed azide-alkylene cycloaddition (CuAAC) (copper-catalyzed reaction of an azide with an alkyne), copper-free azide-alkyne cycloaddition, such as strain-promoted azide-alkyne cycloaddition (SPAAC), Diels-Alder or inverse electron Diels-Alder reaction, thiol-ene or thiol-yne reaction, and alkene-tetrazole photoclick reaction.

The term “targeting ligand” as used herein refers to a ligand that binds to a receptor resulting in receptor-mediated endocytosis. Coupling a targeting ligand to the carrier of the invention allows targeted delivery and hence receptor or even cell specific delivery. For example, by incorporating an azido functional group or a thiol group into the carrier the nanoparticle can be converted to a receptor-targeted nanoparticle, e.g, via copper-free click chemistry for reacting azido functional groups with dibenzocyclooctyne (DBCO)-containing targeting ligands or thiol chemistry including thiol-maleimide addition for coupling targeting ligands such as folic acid (FolA)-PEG for folate receptor α (FRα)-specific delivery.

The term “carrier” as used herein relates to a non-viral carrier for the delivery of nucleic acid and/or protein. In the context of the present invention the carrier is a sequence-defined artificial polymer or lipo-oligomer, more specifically a carrier comprising polar and apolar cationizable domains (such as artificial amino acids (PCS), e.g., Stp, and lipo-amino fatty acids (LAFs)) and that forms nanoparticles, such as a complex (polyplex) with its cargo or a lipid nanoparticle (LNP) loaded with its cargo.

The term “nanoparticle” as used herein relates to small particles in the nanomolar range and include complexes of the carrier (e.g., the sequence-defined lipo-oligomer) formed with its cargo (e.g. mRNA, pDNA or Cas protein/gRNA RNP complex) and can be as small as 6 nm up to several hundreds of nanometers. Such nanoparticles are also sometimes referred to as polyplexes. Nanoparticles also include LNPs. Nanoparticles, both polyplexes and LNPs, are used for cellular delivery and important factors for successful delivery are size, structure, stability, nucleic acid complexation or encapsulation efficiency, cellular uptake and endosomal escape.

The term “lipid nanoparticle (LNP)” are nanoparticles comprising lipids. LNPs are typically spherical and have an average diameter between 10 and 1000 nm. LNPs, such as mRNA vaccines for SARS-CoV-2 (BioNTech: BNT162b2, also known as Comirnaty® and Moderna: mRNA-1273, also known as Spikevax®), are made of four types of lipids, an ionizable cationic lipid (whose positive charge binds to negatively charged mRNA) and helper lipids, including a PEGylated lipid (for stability), a phospholipid (for structure, e.g., 1,2-distearoyl-sn-glycero-3-phosphochloline (DSPC)), and cholesterol (for structure) (see e.g., Albertsen et al., Advanced Drug Delivery Reviews 188, 2022:114416:1-17).

The term “polyplex” as used herein refers to polymer complexes formed by electrostatic interactions between cationic polymers and anionic nucleic acids (e.g., non-viral DNA expression vector, such as pDNA, miRNA or siRNA) or other negatively charged cargo.

Precise sequence-defined artificial lipo-oligomers can be generated to meet the requirements for specific delivery. These sequence-defined artificial lipo-oligomers may be formed by peptide-like artificial macromolecular structures comprising an oligo(alkylamino) acid (also referred to as artificial amino acid) and two or more lipo amino fatty acids (LAF) connected by branching connector(s) to different topologies (e.g., U-shape structure or bundle structure), which may be generated e.g., by solid-phase assisted peptide synthesis (SPPS). The sequence-defined artificial lipo-oligomers, such as the LAF containing carriers according to the invention are also referred to as “carrier” herein. According to the present invention the sequence-defined artificial lipo-oligomers is a carrier comprising at least one polar cationizable domain and two or more apolar cationizable domains connected by branching connectors.

### The Novel Lipo Amino Fatty Acid (LAF) Containing Carrier

The present invention provides a carrier comprising at least one polar cationizable domain (PCD), two or more apolar cationizable domains (ACD) and at least one branching connector (BC), wherein the two or more ACDs are linked by at least one branching connector to at least one PCD, wherein
(a) the PCD
   (i) is an oligo(alkylamino) acid of formula I:

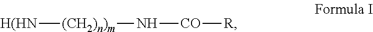

wherein n=2 or 3, alternating 2 and 3 or alternating 3 and 2; and m=3 or 4; and
wherein R is
   —(CH₂)_{y}—CO₂H, wherein y=2, 3 or 4;

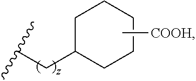

wherein z=0 or 1, such as

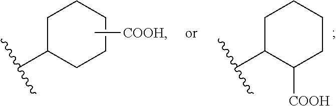

—CH₂-(cyclohexylene)-CH₂—CO₂H;
—CH₂—C(CH₃)₂—CH₂—CO₂H or CH₂—C(CH₂—CH₃)₂—CH₂—CO₂H;
—CH₂—O—CH₂—CO₂H; or
—CH₂—NH—CH₂—CO₂H; or
(ii) is an epsilon-poly-L-lysine comprising structure, preferably an (ε-lysine)ₙ 6-amino hexanoic acid ((^{ε}K)ₙ-6-Ahx), (ε-lysine)ₙ₊₁ ((^{ε}K)ₙ₊₁) or (ε-lysine)ₙ ((^{ε}K)ₙ), wherein n=2, 3 or 4, preferably 2 or 3;
(b) the BC is a bisamide connector selected from the group consisting of L-lysine, L-ornithine and an artificial amino acid comprising two amino groups and a carboxyl group; and
(c) the ACD is a lipo amino fatty acid (LAF) comprising a tertiary amine of formula II:

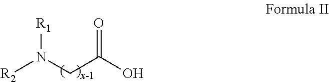

wherein x is 4-12, and R1 and R2 is an acyclic alkane or alkene, wherein R1 is C6-C16 and R2 is C1-C16 and wherein R1 and R2 may be the same or different.

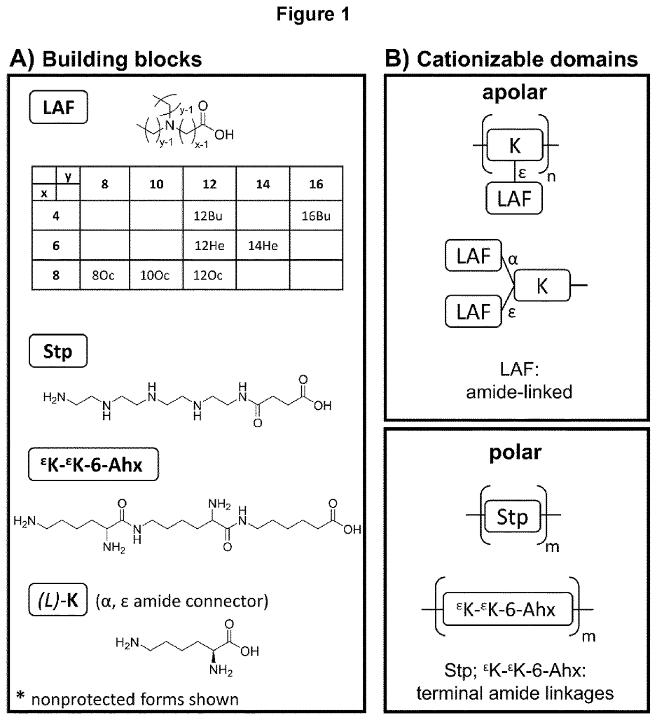

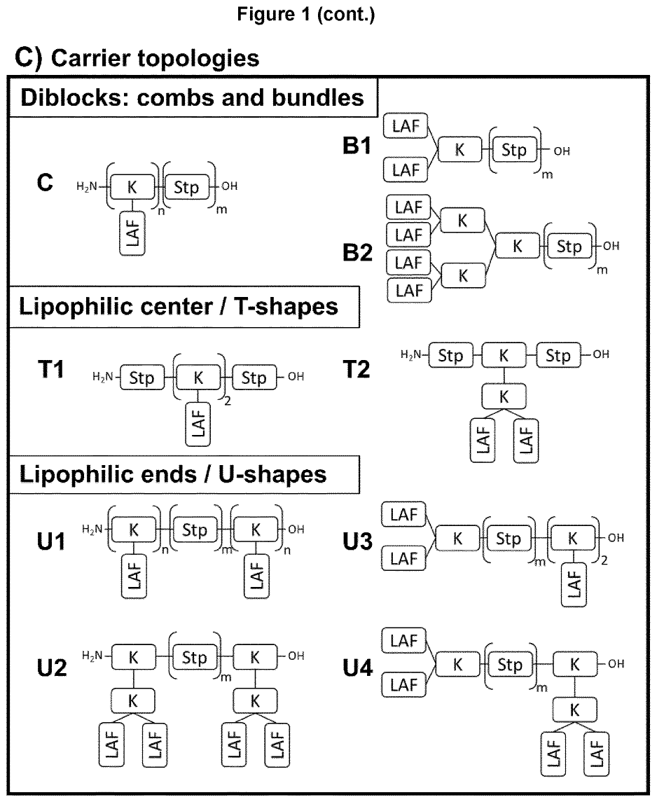

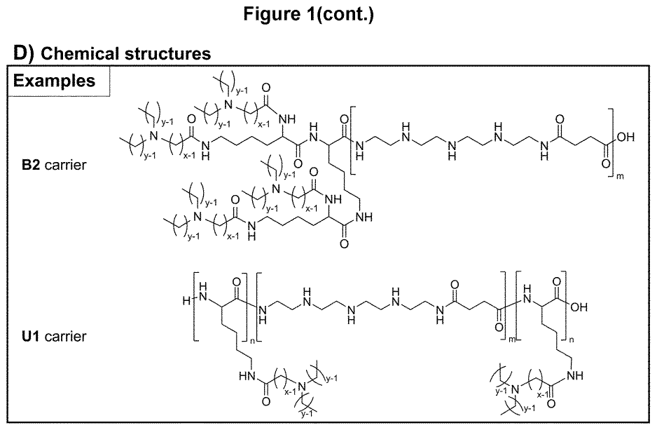

The carrier comprises a novel combination of building blocks combining hydrophilic building blocks (PCDs) and lipophilic building blocks (ACDs), which are both cationizable, comprising two or more tertiary amine groups amenable to pH specific protonation. The C-terminal carboxyl group of the PCD may further be modified or further coupled. The branching connector(s) (BC) allow(s) linking the PCDs and ACDs into structures of various topologies and/or ratios. The at least one PDC, the two or more ACDs and the at least one BC are covalently linked via amid bonds. The person skilled in the art would understand considering the structure of the PCDs, ACDs and BCs that the amid bond is formed between a carboxyl group and an amino group of the respective building blocks (amide-linked, FIG. 1B) and hence may be, e.g., an amide bond formed by an α-carboxyl and an α-amino group (such as in a peptide bond) or another amid bond, such as formed by an α-carboxyl and an ε-amino group (e.g., in lysine) or an δ-amino group (e.g., in ornithine). The carrier comprises typically 1, 2 or 3 PCDs, preferably 1 or 2 PCDs and 2, 4 or 8 ACDs, preferably 2 or 4 ACDs. In certain embodiments the PCD to ACD ratio is between 1:2 to 1:4. Depending on the topology, the optimal ratio may be 1:2 for U-shapes (wherein PCD is one or two) and/or 1:2 to 1:4 for B2 structures, such as 1:4 for B2 structures (wherein PCD is one) or 1:2 for B2 structures (wherein PCD is two). In some embodiments, the carrier is produced by solid phase synthesis. Thus, the PCD:ACD content may be 1:2 or 2:4 for U-shape structures (more specifically 1 PCD and 2 ACDs, or 2 PCDs and 4 ACDs) and/or 1:4 or 2:4 for B2 structures (more specifically 1 PCD and 4 ACDs, or 2 PCDs and 4 ACDs). The PCD:ACD ratio as used herein, if not otherwise stated, refers to the smallest ratio, i.e., a PCD:ACD ratio of 1:2 encompasses an PCD:ACD content of 1:2 and 2:4. Thus, in case a ratio of 2:4 is used, such as in the example, the person skilled in the art would immediately understand that this refers to a PCD:ACD content, i.e., defining the number of PCDs and the number of ACDs, respectively, within the carrier.

The branching connector (BC) is a bisamide connector selected from the group consisting of L-lysine, L-ornithine and an artificial amino acid comprising two amino groups and a carboxyl group. The person skilled in the art would understand that, e.g., the bisamide connector L-lysine is an α, ε-amide connector, while L-ornithine is an α, δ-amide connector. In principle the branching connector may be any amino acid molecule comprising two amino groups and a carboxyl group, including artificial amino acids. Depending on the topology one or more BC may be required in order to link the PCDs and the ACDs. Thus, the two or more ACDs are linked by at least one branching connector to at least one PCD, wherein each ACD may be linked by one or more BC to a PCD. Particularly where a BC is linked to two ACDs, a further BC is needed for connecting the ACD to the PCD. Also, two BCs each carrying a single ACD may be linked to the PCD. The different building blocks are linked via amid bonds to each other (amide-linked) as may be taken from FIG. 1B.

The carrier according to the invention may further comprise a disulfide building block between the at least one PCD and the two or more ACDs and/or a spacer between the at least one PCD and the two or more ACDs. For example, a glycine or any other inert linker between two BCs or between a BC and an ACD or a PCD may serve as a spacer. A disulfide-building block may consist of succinyl-cystamine (P. Klein et al., Nanoscale. 2016, 8(42):18098-18104; S. Berger et al., Biomacromolecules 2021, 22, 1282). Located between the PCD and the ACD it may further facilitate redox-sensitive release of the nucleic acid cargo in the cytosol (A. Krhac-Levacic et al., J. Control Release 2021, 339, 27-40).

### The Apolar Cationizable Domain

According to the invention a cationizable tertiary amine is placed into an apolar lipidic domain to form an apolar cationizable domain (ACD). The ACD is a lipo amino fatty acid (LAF) comprising a tertiary amine of formula II:

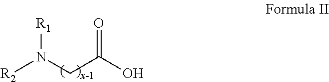

wherein x is 4-12, and R1 and R2 are an acyclic alkane or alkene, wherein R1 is C6-C16 and R2 is C1-C16 and wherein R1 and R2 may be the same or different.

A lipo amino fatty acid (LAF) is a tertiary amine linked to a fatty acid and two acyclic hydrocarbon chains (R1 and R2) (e.g., acyclic alkyl/alkene chains). The nomenclature of LAFs as used herein uses a number and a two letter abbreviation, wherein the number (8, 10, 12, 14, 16) expresses the length of the terminal hydrocarbon chains (if not otherwise specified alkyl chains, also referred to as R1 or R2 in Formula II) and the two letters represent the used amino fatty acid (Oc”, 8-aminooctanoic acid; “He”, 6-aminohexanoic acid; “Bu”, 4-aminobutanoic acid) (also defined by x-1 in Formula II). In Formula II, x may be 4-12, e.g., 4 (4-aminobutanoic acid), 6 (6-aminohexanoic acid), 8 (8-aminooctanoic acid), 10 (10-aminodecanoic acid) or 12 (12-aminododecanoic acid), preferably 4-10, e.g., 4, 6, 8 or 10, more preferably 4-8, e.g., 4, 6 or 8, even more preferably 6-8, e.g., 6 or 8. Further, R1 and R2 in Formula II each are independently an acyclic alkane or alkene, wherein R1 is C6-C16 and R2 is C1-C16 and wherein R1 and R2 may be the same or different. Preferably R1 and R2 are an acyclic alkane. Without being bound by theory, it is believed that at least one long hydrocarbon chain (2 C6) is required while the other hydrocarbon chain may be shorter. It is therefore not necessary that R1 and R2 are the same. The person skilled in the art would therefore understand that the apolar cationizable domain may be a symmetrical or asymmetrical lipophilic amine. Yet, the synthesis of the LAF is simpler if R1 and R2 are the same and hence the generated APCs are symmetrical dialkyl-amino fatty acids (lipoamino fatty acids). Thus, preferably R1 and R2 are the same and/or are an acyclic alkane or alkene of C6-16, preferably an acyclic alkane of C6-C16.

Overall, the length of the terminal hydrocarbon chains (R1 and R2) seems to be more important compared to the length of the amino fatty acid. However, an overall size of the LAF of C16 to C22, preferably C16 to C20 (e.g., 12Oc), interrupted by the tertiary amine seems to be advantageous. The optimal size of the LAF may vary with the topology. For example, a slight preference for LAFs with an overall size of C20 (e.g., 12Oc) was found for U-shape structures, particularly U1 structures, and a preference for C16 (e.g., 8Oc) was found for B2 structures. In certain embodiments, the LAF has an overall length of C18-C20 (e.g., 12He, 14He or 12Oc) for U-shape structures, particularly C20 (e.g. 14He or 12Oc) for U1 structures. Thus, in the ACD x is 4-12, preferably 6-10, more preferably 6-8, and R1 and R2 are C6-16, preferably C8-C14, more preferably 12-14. In certain embodiments, the LAF has an overall length of C16-C18 (e.g., 8Oc, 12Bu, 10Oc or 12He), preferably C16 (e.g., 8Oc or 12Bu) for B2 structures. Thus, in the ACD x is 4-10, preferably 4-8, and R1 and R2 are C6-14, preferably C8-C12. The nomenclature used herein for LAFs is as follows: The number (8, 10, 12, 14, 16) refers to carbon atoms (C) and expresses the length of the terminal alkyl chains; the two letters represent the used amino fatty acid (“Oc”, 8-aminooctanoic acid; “He”, 6-aminohexanoic acid; “Bu”, 4-aminobutanoic acid). The overall length refers to the sum of the carbon atoms (C) in the terminal alkyl chain and the amino fatty acid.

It has been observed previously that the chain length of saturated fatty acids modulates the lytic activity of lipo-oligoaminoamides. The highest membranolytic activity was found for carbon chain lengths of C8 to C14, the lowest for fully saturated C18 chains (S. Berger et al. Biomacromolecules 2021, 22, 1282). Introduction of double bonds or hydroxylation in the mid of the chain favorably changed the fusogenic potential of carriers. Hydroxystearic acid, for example, was found to be superior to stearic acid regarding delivery of Cas9/sgRNA ribonucleoprotein (S. Reinhard et al., Chem Med Chem 2017, 12, 1464; J. Kuhn et al., Bioconjugate Chem 2020, 31, 729-742). We therefore hypothesized that introducing a cationizable tertiary amine into the center of a hydrocarbon fatty acid might drastically change the lytic activity upon reversible cationization.

Moreover, incorporation of fatty acids in nucleic acid carriers such as lipo-oligoaminoamides promote chain-length dependent nanoparticle stabilization due to hydrophobic interactions. The longer the fatty acid chain length, the more stable the nanoparticles. However, shorter fatty acids with lengths around C6 to C10 were figured out to be more beneficial for transfection efficiency (S. Berger et al., Biomacromolecules 2021, 22, 1282), suggesting that an optimal balance between extracellular nanoparticle stability and sufficient intracellular cargo release has to be found. A polar amide bond in the center of a C18 chain resulted in similar behavior of the nanocarrier in terms of nanoparticle stability and transfection efficiency than shorter C9 fatty acid (A. Krhač Levačić et al., Journal of controlled release: 2021, 339, 27). Therefore, the hydrophobic character of the fatty acid was reversibly disrupted in a pH-dependent manner by introducing a cationizable tertiary amine.

Isolated tertiary amino groups have a high pKa value far above neutrality, but this is strongly dependent on the surrounding microenvironment. The concept of reversible protonation/deprotonation of tertiary amines in hydrophobic environment was applied to alter the hydrophobic character of the lipidic domain within the carriers in a dynamic pH-dependent manner. By this, the carriers and corresponding nucleic acid nanoparticles may adapt to the microenvironment like chameleons, switching between water-solubility and -insolubility in dependence on their protonation state. Additionally, nanoparticle stability might be reduced upon protonation of the tertiary amines due to less hydrophobic interactions. All of this, together with the enhanced membranolytic activity upon protonation, is believed to be helpful in terms of membrane transfer and effective cargo release at its site of action.

The novel LAF carriers comprise a polar cationizable domain (PCD), e.g. one or more succinyl-tetraethylenepentamine (Stp) units, and two or more apolar cationizable domain (ACD) consisting of the novel lipo amino fatty acids (LAFs). These domains are covalently connected via branching connector, such as lysines, in order to form different topologies with varying position of the responsive domains, resulting in hydrophilic/lipophilic diblocks (i.e., combs and bundles), T-shapes with lipophilic center, and U-shapes with lipophilic ends (FIG. 1). An important consideration was found to be the ratio of PCD to ACD units (e.g., Stp to LAF units) and thus the hydrophilic/lipophilic balance. Standard Fmoc solid-phase assisted peptide synthesis was utilized to generate the library of sequence-defined carriers tested in the Examples. The synthetized and tested structures are listed in Tables 1.1-1.3. The identity of the carriers was proved via MALDI (matrix-assisted laser desorption/ionization) mass spectrometry and 1H-NMR (nuclear magnetic resonance) spectroscopy. The novel LAF building blocks were obtained by reductive amination of different amino fatty acids with fatty aldehydes of various lengths and were analyzed via ESI (electron spray ionization) mass spectrometry and 1H-NMR spectroscopy (for analytical data see methods section). Commercially non-available fatty aldehydes (i.e., tetradecanal, hexadecanal) were generated from corresponding alcohols and confirmed by EI (electron ionization) mass spectrometry, ¹H-NMR, and ¹³C-NMR spectroscopy (for analytical data see methods section). Upon variation of the carbon chain lengths of both amino fatty acids and fatty aldehydes, the position of the tertiary amine within the LAFs is altered, which might have an impact on the lytic activity of the corresponding carriers. The synthesized LAF carriers were evaluated in terms of physicochemical properties and their ability to efficiently complex and deliver different nucleic acid cargos as polyplexes (non-viral DNA expression vector (e.g., pDNA), mRNA, mRNA/pDNA, siRNA and Cas mRNA/sgRNA or Cas mRNA/sgRNA/ssDNA) or LNPs (mRNA and siRNA).

### The Polar Cationizable Domain

The PCD comprised in the carrier according to the invention is an artificial amino acid, more specifically an oligo(alkylamino) acid, or an epsilon-poly-L-lysine with or without a terminal δ-amino hexanoic acid (6-Ahx). In certain embodiments the at least one PCD is an oligo(alkylamino) acid of formula I:

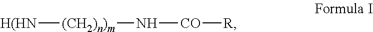

wherein n=2 or 3, alternating 2 and 3 or alternating 3 and 2; and m=3 or 4; and wherein R is
   —(CH₂)_{y}—CO₂H, wherein y=2, 3 or 4;

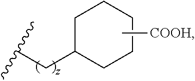

wherein z=0 or 1, such as

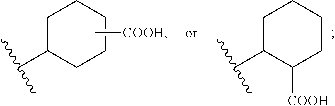

—CH₂-(cyclohexylene)-CH₂—CO₂H;
—CH₂—C(CH₃)₂—CH₂—CO₂H or CH₂—C(CH₂—CH₃)₂—CH₂—CO₂H;
—CH₂—O—CH₂—CO₂H; or
—CH₂—NH—CH₂—CO₂H;
preferably, R is
   —(CH₂)_{y}—CO₂H, wherein y=2, 3 or 4; or
   —CH₂—NH—CH₂—CO₂H.

The oligo(alkylamino) acid of formula I contains 3 or 4 (m=3 or 4) protonatable alkylamino repeats, particularly 3 or 4 repeats of protonatable amino ethylene (HN—(CH₂)₂; i.e., n=2) or amino propylene motifs (HN—(CH₂)₃; i.e., n=3) or alternating amino ethylene and amino propylene motifs (n=2 and 3, preferably alternating) or vice versa. In certain embodiments n is 2, 2, 2 (for m=3) or 2, 2, 2, 2 (for m=4), or n is 3, 3, 3 (for m=3) or 3, 3, 3, 3 (for m=4), or n is 2, 3, 2 (for m=3) or 2, 3, 2, 3 (for m=4), or n is 3, 2, 3 (for m=3) or 3, 2, 3, 2 (for m=4).

In certain embodiments, the oligo(alkylamino) acid is a tetraethylenepentamine or a triethylenetetramine of formula Ib:

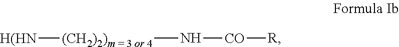

preferably a tetraethylenepentamine of formula Ic:

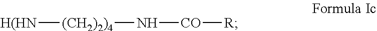

wherein R is
   —(CH₂)_{y}—CO₂H, wherein y=2, 3 or 4;

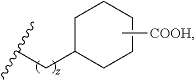

wherein z=0 or 1, such as

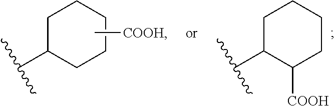

—CH₂-(cyclohexylene)-CH₂—CO₂H;
—CH₂—C(CH₃)₂—CH₂—CO₂H or CH₂—C(CH₂—CH₃)₂—CH₂—CO₂H;
—CH₂—O—CH₂—CO₂H; or
—CH₂—NH—CH₂—CO₂H;
preferably, R is
   —(CH₂)_{y}—CO₂H, wherein y=2, 3 or 4; or
   —CH₂—NH—CH₂—CO₂H.

In certain embodiments, the oligo(alkylamino) acid is selected from the group consisting of the following formulas:

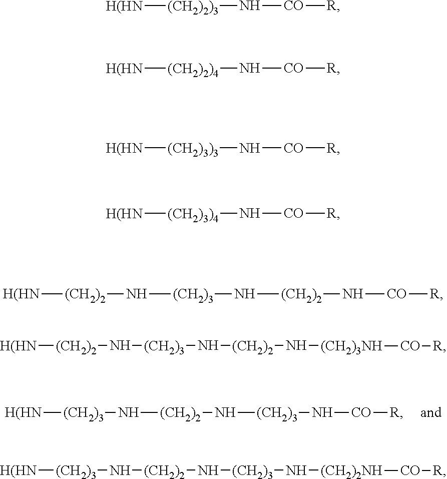

wherein R is as defined for formula I.

Suitable oligo(alkylamino) acids according to the invention without being limited thereto are succinyl-tetraethylenepentamine (Stp), 1,2-cyclohexanedicarboxyl-tetraethylenepentamine (Htp), phthalyl-tetraethylenepentamine (Ptp), naphthalenedicarboxyl-tetraethylenepentamine (Ntp), glutaryl-tetraethylenepentamine (Gtp), 1,1-cyclohexanediacetyl-tetraethylenepentamine (chGtp), iminodiacetyl-tetraethylenepentamine (IDAtp), glutaryl-triethylenetetramine (Gtt), glutaryl-3,3-ethylenedipropylenetetramine (GEIPA), diglycolyl-tetraethylenepentamine (dGtp), succinyl-triethylenetetramine (Stt), dimethyl-glutaryl-tetraethylenepentamine (dmGtp) or diethyl-glutaryl-tetraethylenepentamine (deGtp) having the following formulas:

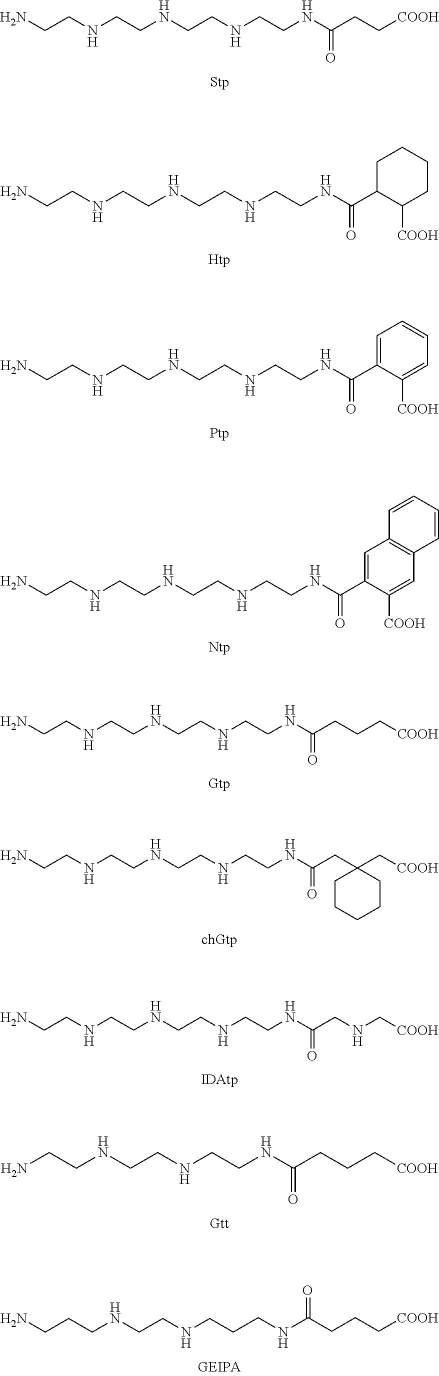

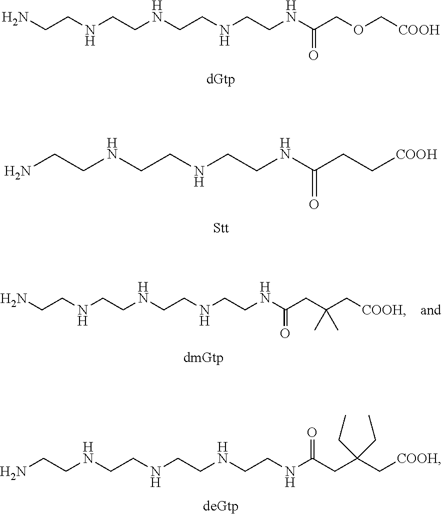 [SMILES: NCCNCCNCCNCCNC(=O)COCC(=O)O ; NCCNCCNCCNC(=O)CCC(=O)O ; CC(C)(CC(=O)O)CC(=O)NCCNCCNCCNCCN ; "and" ; CCC(CC)(CC(=O)O)CC(=O)NCCNCCNCCNCCN]

preferably selected from the group consisting of:

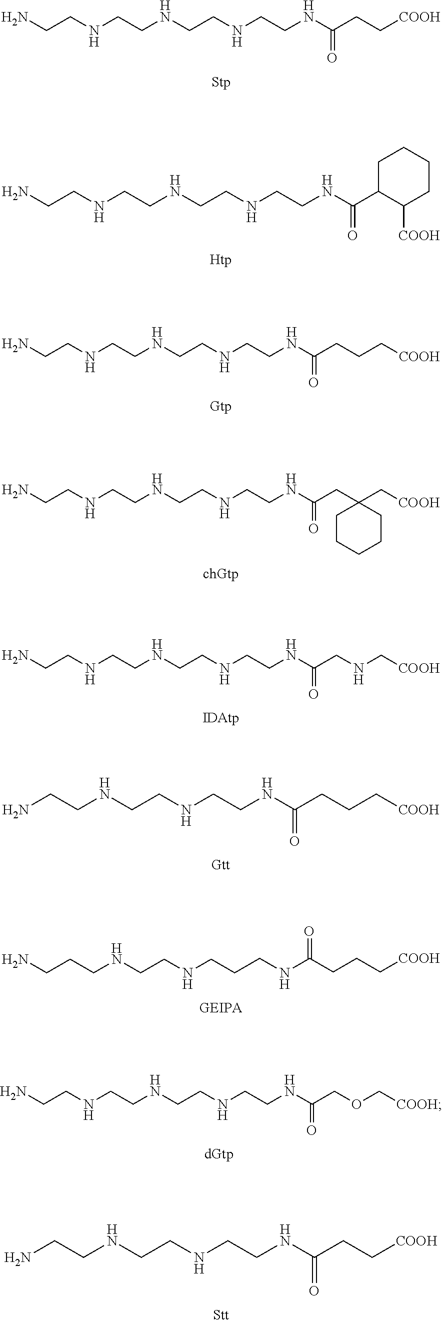

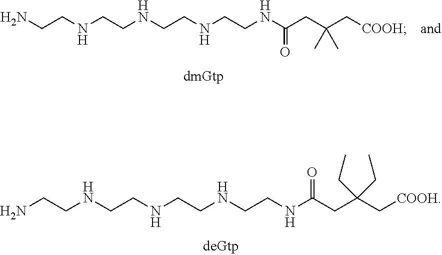 [SMILES: CC(C)(CC(=O)O)CC(=O)NCCNCCNCCNCCN ; "and" ; CCC(CC)(CC(=O)O)CC(=O)NCCNCCNCCNCCN]

More preferably the oligo(alkylamino) acid is selected from the group consisting of Stp, Htp, Gtp, chGtp, dGtp and dmGtp, even more preferably Stp, Htp, chGtp or dmGtp, even more preferably Stp. The definitions for “R” as used herein are according to commonly used nomenclature. The residue “—CH₂-(cyclohexylene)-CH₂—CO₂H” as used herein may also be referred to as “—CH₂—C(CH₂—CH₂—CH₂—CH₂—CH₂)—CH₂—CO₂H”, and is preferably “—CH₂-[(1,1)-cyclohexylene]-CH₂—CO₂H”, and encompasses the respective residue of e.g., chGtp.

The oligo(alkylamino) acids are typically synthesized as protected building blocks prior to generating the (sequence-defined) carrier comprising two or more ACDs (LAFs). The person skilled in the art would know suitable protection groups which include Fmoc and Boc. Thus, the oligo(alkylamino) acids in their protected form may be Fmoc-Stp(Boc₃)-OH, Fmoc-Gtp(Boc₃)-OH, Fmoc-IDAtp(Boc₃)-OH, Fmoc-Gtt(Boc₂)-OH, Fmoc-GEIPA(Boc₂)-OH or Fmoc-dGtp(Boc₃)-OH etc.

In alternative embodiments the at least one PCD is an epsilon-poly-L-lysine comprising structure, preferably epsilon-poly-L-lysine-6-amino hexanoic acid, more specifically an (ε-lysine)ₙ-6-amino hexanoic acid ((^{ε}K)ₙ-6-Ahx), wherein n=2, 3 or 4, or an epsilon-poly-L-lysine, more specifically an (ε-lysine)ₙ₊₁ ((^{ε}K)ₙ₊₁) or an (ε-lysine)ₙ ((^{ε}K)ₙ), wherein n=2, 3 or 4. In case of more than one PCD, the PCDs may be directly linked by an amide linkage or may be linked by a branching connector, such as L-lysine or L-ornithine. In certain alternative preferred embodiment the at least one PCD is (^{ε}K)ₙ-6-Ahx or (εK)ₙ₊₁, wherein n=2 or 3. Thus, the epsilon-poly-L-lysine-δ-amino hexanoic acid may be εK-εK-6-Ahx, εK-εK-εK-6-Ahx or εK-εK-εK-εK-6-Ahx, preferably εK-εK-6-Ahx, εK-εK-εK-6-Ahx. Aminocaproic acid (6-amino hexanoic acid, ε-Ahx) is a derivative and analogue of lysine. Thus, analogously the PCD may be εK-εK, εK-εK-εK, εK-εK-εK-εK, or εK-εK-εK-εK-εK, preferably εK-εK-εK or εK-εK-εK-εK, more preferably εK-εK-εK. In a specific embodiment the at least one PCD is εK-εK ((^{ε}K)₂), more preferably the at least one PCD are two PCDs and the PCDs are two (^{ε}K)₂ linked by a branching connector, such as L-lysine. In certain preferred embodiments the at least one PCD is εK-εK-6-Ahx of which the structure is shown below:

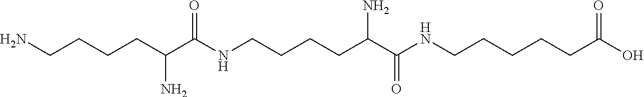 [SMILES: NCCCCC(N)C(=O)NCCCCC(N)C(=O)NCCCCCC(=O)O]

The synthesized LAF carriers mainly comprise succinyl-tetraethylenepentamine (Stp) as PCD building block (Tables 1.1 to 1.3), but also LAF carriers comprising Stp analogs (Table 2) or epsilon-poly-L-lysine comprising structures as polar cationizable domain have been synthesized (Tables 1.3 and 3.3, e.g. ID 1747 and 1851). However, tripeptide cationizable Stp analoga, such as (ε-lysine)₂-δ-amino hexanoic acid, have also been shown to be effective. Similar results are expected for (ε-lysine)₃ and (^{ε}K)ₙ-6-Ahx) or (^{ε}K)ₙ₊₁ analoga±^{ε}K. Moreover, other oligo(alkylamino) acids have proven effective in the context of other sequence-defined lipo-oligomers and are therefore expected to be effective also for the carriers of the present invention. In certain embodiments, the C-terminal carboxyl group of the PCD may be modified or further coupled. The optimal LAF, PCD, topology and LAF:PCD ratio may be optimized for each specific cargo.

### Carrier Topology

The branching connector(s) (BC) allow(s) linking the PCDs and ACDs to form structures of various topologies and/or ratios. In certain embodiments the PCD to ACD ratio is between 1:2 to 1:4. The carrier may have a topology with a diblock structure, such as combs and bundles, with a lipophilic center, such as in T-shape structures, or with lipophilic ends, such as in U-shape structures. More specifically, the carrier may have one of the following topologies:
diblocks/combs and bundles

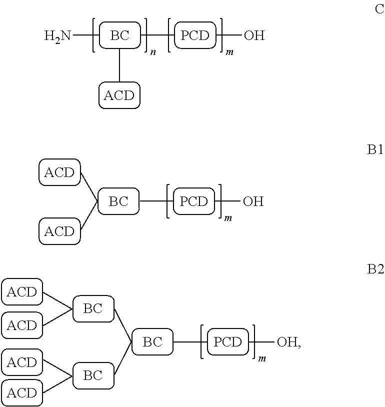

wherein m=1, 2, 4 and n=2, 4, 6 for combs, and m=1, 2 or 3 for bundles;
lipophilic center/T-shapes

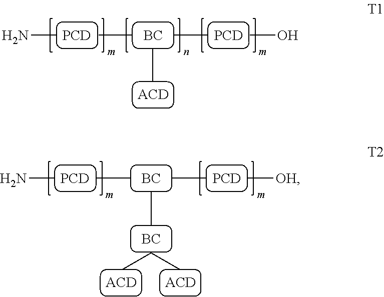

wherein m=1 or 2 and n=2; or
lipophilic ends/U-shapes

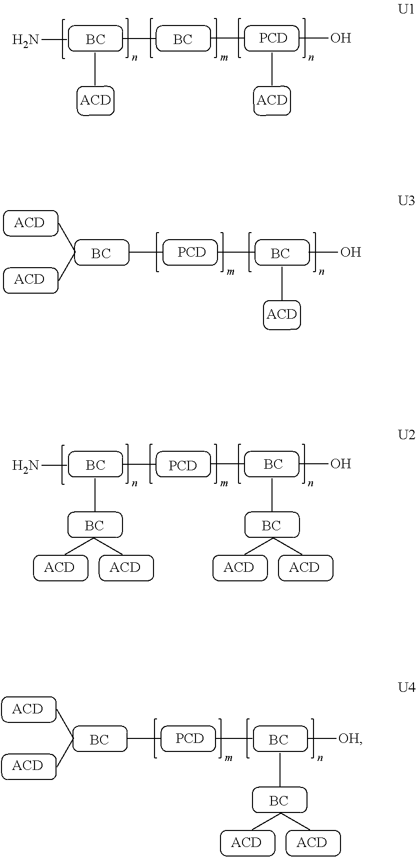

wherein m=1, 2 or 3 and n=1 or 2, preferably m=1 or 2 and n=1 or 2.

In certain embodiments, the carrier has a topology selected from the group consisting of: A bundle structure having the structures B1 or B2:

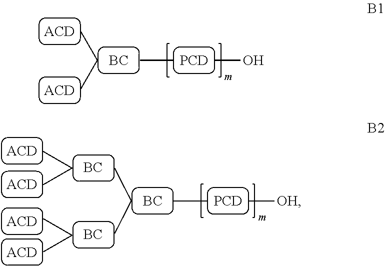

wherein m=1, 2 or 3; and
a U-shape structure having the structures U1, U2, U3 or U4

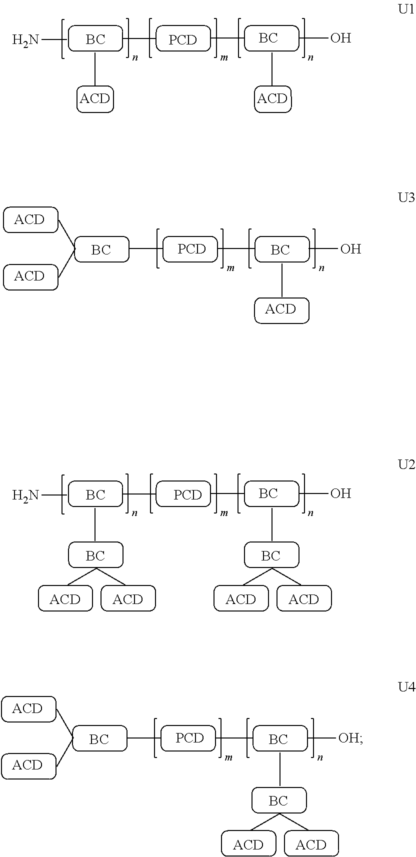

wherein m=1, 2 or 3 and n=1 or 2 and, preferably m=1 or 2 and n=1 or 2. Preferably, in U-shape structures, both lipophilic ends comprise the same number of ACDs and/or one or two ACDs at each end. Thus, for U1 n=1 or 2, for U2 n=1, for U3 n=2 and for U4 n=1. Two ACDs at either end means either two BC-ACD subunits or one BC-BC-(ACD)₂ subunit. In certain embodiments the PCD to ACD ratio in U-shape structures is 1:2 to 1:4, preferably 1:2, wherein the carrier preferably has a PCD:ACD content of 1:2 or 2:4 (more specifically 1 PCD and 2 ACDs or 2 PCDs and 4 ACDs). Thus, for U1 m=1 and n=1 or m=2 and n=2, preferably m=1 and n=1; for U2 m=2 and n=1, for U3 m=2 and n=2 and for U4 m=2 and n=1. In certain embodiments the LAF has an overall size of C18-C20 (e.g., 12He, 14He or 12Oc) for U-shape structures, particularly C20 (e.g. 14He or 12Oc) for U-shape structures, preferably U1 structures. Thus, in the ACD x may be 4-12, preferably 6-10, more preferably 6-8 and R1 and R2 may be C6-16, preferably C8-C14, more preferably 12-14. In bundle structures PCD to ACD ratio is preferably 1:2 in B1 structures and 1:2 or 1:4 in B2 structures (wherein the carrier has a PCD:ACD content of 1:2 in B1 structures and 2:4 or 1:4 in B2 structures). Thus, preferably m=2 for B1 and m=1 or 2 for B2 structures. More preferably the bundle structure is a B2 structure with an PCD to ACD ratio of 1:2 or 1:4 (and a PCD:ACD content of 2:4 or 1:4), preferably an PCD to ACD ratio of 1:4 (and a PCD:ACD content of 1:4). In certain embodiments the LAF has an overall size of C16-C18 (e.g., 8Oc, 12Bu, 10Oc or 12He), preferably C16 (e.g., 8Oc or 12Bu) for B structures, preferably B2 structures. Thus, in the ACD x may be 4-10, preferably 4-8, and R1 and R2 may be C6-14, preferably C8-C12.

The person skilled in the art will understand that the carrier according to the invention may further comprises a disulfide building block between the at least one PCD and the two or more ACDs, and/or a spacer between the at least one PCD and the two or more ACDs. The disulfide building block may e.g. be succinyl-cysteamine (P. M. Klein et al. Nanoscale 2016, 8, 18098). The spacer may e.g., be a glycine or a 6-aminohexanoic acid. The free C-terminal carboxyl group of the PCD within the carrier may further be modified or further coupled. The carrier of the present invention may further comprise in addition a terminal functional group. The functional group may be used for coupling a further molecule, such as a targeting ligand or cargo (particularly a PMO), preferably via click-chemistry. In certain embodiments, the carrier comprises a terminal functional group selected from an azido-group and a thiol group, preferably a terminal azido-lysine (K(N3)), azido-hexane, cysteine or homocysteine. The term “terminal” as used herein means the free amino group (N-terminus) or carboxyl group (C-terminus). While the bundle structures (B1 and B2) and the U3 and U4 U-shape structures only have a free carboxyl group, the U1 and U2 U-shape structures have a free amino group and a free carboxyl group. Thus, in U1 and U2 U-shape structures the terminal reactive group may be at the N-terminus and/or the C-terminus, preferably at the N-terminus. Although a single terminal functional group (azido-group or thiol-group) is sufficient, theoretically the carrier may also comprise an azido-group at the N-terminus and/or the C-terminus (if both a free amino group and a free carboxyl group are available). Preferably the functional group is an azido-group, which allows coupling to a further molecule using click chemistry. More preferably the carrier comprising a terminal azido-group is coupled to the further molecule via click chemistry, more preferably click chemistry with a dibenzocylooctyne-coupled targeting ligand.

Click chemistry is a general term for highly specific, in many cases biorthogonal, covalent conjugation reactions, that are modular, efficient and relatively insensitive to solvent parameters, water and oxygen. Various click chemistry reactions are known in the art and the person skilled in the art would know how to select a specific click chemistry reaction for a certain conjugation and particular for a certain protein or oligomer, such as the carrier according to the invention. Typical click reactions, without being limited thereto, are copper-catalyzed azide-alkylene cycloaddition (CuAAC) (copper-catalyzed reaction of an azide with an alkyne), copper-free azide-alkyne cycloaddition, such as strain-promoted azide-alkyne cycloaddition (SPAAC), Diels-Alder or inverse electron Diels-Alder reaction, and alkene-tetrazole photoclick reaction. Alternatively, a single terminal cysteine may be used for coupling via disulfide bond formation, thiol-maleimide or other thiol click reaction, and preferably a targeting ligand is coupled to the cysteine.

The targeting ligand may be a ligand that binds to a receptor resulting in receptor-mediated endocytosis (also referred to as receptor-mediated internalization). This may be a natural ligand or an artificial ligand, such as an antibody a fusion protein or a small molecule binding to a receptor and mediating uptake. Coupling a targeting ligand to the carrier of the invention allows targeted delivery and hence receptor or even cell specific delivery. For example, by incorporating an azido functional group into the sequence-defined oligomer the carrier or nanoparticle can be converted to a receptor-targeted carrier or nanoparticle, e.g, via copper-free click chemistry with dibenzocyclooctyne (DBCO)-containing targeting ligands, such as folic acid (FolA)-PEG as targeting ligand for folate receptor α (FRα)-specific delivery. Alternatively, a single terminal thiol group in combination with thiol chemistry may be used for coupling targeting ligands, such as folic acid (FolA)-PEG as targeting ligand for folate receptor α (FRα)-specific delivery. For example, targeting ligands such as mannose, cRGD, folic acid, methotrexate (MTX), c-Met-binding peptide (cMBP2), transferrin (Tf), AP-1, EGF, EGF receptor-binding peptide (GE11) and IL-6 receptor binding 16P7 peptide may be used. Other suitable receptors for receptor-mediated internalization and specific delivery may, e.g., be tumor antigens and the like.

The carrier may further comprise one or more histidine, cysteine and/or arginine. Particularly histidine and/or arginine may be between PCDs and/or adjacent to PCDs. Without being bound by theory, histidines or other imidazole derivatives with a pKa of around 6 have been incorporated into oligomers as they increase their endosomal buffer capacity, which may result in improved endosomal escape and delivery. Cysteine may help to stabilize the nanoparticle by formation of disulfide bonds. Particularly for less stable carriers and/or nanoparticles formed by such carriers cysteines, particularly two cysteines at terminal positions of each carrier that can form disulfide bonds between two carriers, may be advantageous.

Without being limited thereto, the carriers according to the invention are preferably produced using solid phase synthesis. The carriers according to the invention are particularly suitable for nucleic acid and/or protein delivery, preferably for nucleic acid delivery. The carriers are suitable for in vitro, in vivo or ex vivo nucleic acid and/or protein delivery.

Thus, in a further aspect, the present invention relates to the use of a carrier according to the invention for nucleic acid and/or protein delivery, preferably for nucleic acid delivery. More specifically, the carrier is for delivery of nucleic acid and/or protein into a target cell. In certain embodiments, the nucleic acid and/or protein delivery is in vitro nucleic acid and/or protein delivery or ex vivo nucleic acid and/or protein delivery, i.e., delivery to cells in cell culture.

### Nanoparticles and Therapeutic and Non-Therapeutic Uses Thereof

The present invention further relates to a nanoparticle comprising the carrier of the invention, further comprising a cargo, wherein the cargo comprises a nucleic acid and/or a protein, preferably a nucleic acid. In certain embodiments the cargo comprises a nucleic acid and/or a protein, preferably a nucleic acid. Preferably the cargo comprises RNA or DNA. More preferably the cargo comprises mRNA, Cas mRNA/gRNA, siRNA, miRNA (or other mediators of RNAi), polyinosinic: polycytidylic acid (poly(I:C)), phosphodiamidate-morpholino-oligomer (PMO), a non-viral DNA expression vector, such as pDNA, Cas protein/gRNA ribonucleoprotein (RNP) or mixtures thereof. Even more preferably the cargo comprises mRNA, Cas mRNA/gRNA, siRNA, a non-viral DNA expression vector (e.g., pDNA) or a combination thereof. The Cas mRNA/gRNA may further comprise donor DNA and hence the cargo may be Cas mRNA/gRNA/ssDNA.

In contrast to other ionizable lipids, the carrier of the present invention contains two or more ionizable apolar domains in addition to one or more separate polar ionizable domains.

The nanoparticle according to the invention may be a complex (polyplex) formed by the carrier mixed with the cargo, a lipid nanoparticle (LNP) comprising the carrier loaded with the cargo, or a complex formed by the cargo covalently coupled to the carrier. In the LNP the carrier serves as an ionizable lipid that is used together with further lipid components referred to as helper lipids, including a PEGylated lipid (e.g., 1,2-dimyristoyl-rac-glycero-3-methoxypolyethylene glycol-2000 (DMG-PEG 2000)), a phospholipid (e.g., 1,2-distearoyl-sn-glycero-3-phosphochloline (DSPC)) and cholesterol. Other ionizable lipids used are e.g., ALC-0315 (Comirnaty®) and SM-102 (Spikevax®) in the COVID-19 LNP vaccines or MC3 in Onpattro®. LNPs are particularly suitable for delivery of RNA cargo, such as mRNA and siRNA.

Complexes formed by electrostatic interactions between lipo-oligomers (cationic polymer) and the cargo (e.g., negatively charge nucleic acid) are referred to as polymer complexes or polyplexes. They are formed by mixing the carrier and the cargo. Polyplexes are suitable for delivery of any cargo comprising RNA or DNA, RNA or DNA. More preferably the cargo comprises mRNA, Cas mRNA/gRNA, siRNA, miRNA (or other mediator of RNAi), polyinosinic:polycytidylic acid (poly(I:C), a non-viral DNA expression vector (e.g., pDNA), Cas protein/gRNA ribonucleoprotein (RNP) or mixtures thereof. The carrier and the cargo are preferably mixed at a lipo-oligomer nitrogen (N) to nucleic acid phosphate (P) ratio (N/P ratio) of about 1:12 to 1:30. Only the protonatable nitrogens of the PCD and of the ACD are considered for determining the N/P ratio. In an oligo(alkylamino) acid with five nitrogens, three are protonatable and with 4 nitrogens, two are protonatable. A lipoamino fatty acid (LAF) contains one protonatable nitrogen. The number of protonatable nitrogens of investigated carriers are listed in Tables 1.1 to 1.3.

The carrier of the present invention may further comprise a terminal functional group for coupling cargo, particularly phosphodiamidate-morpholino-oligomers (PMOs), preferably via click-chemistry. In certain embodiments, the carrier comprises a terminal functional group selected from an azido-group and a thiol group, preferably a terminal azido-lysine (K(N₃)), azido-hexane, cysteine or homocysteine. Preferably PMO is covalently coupled via click-chemistry to an azido-group in the carrier, more preferably dibenzocylooctyne-coupled PMO is covalently coupled via click-chemistry to an azido-group in the carrier. PMO is an oligomer molecule of a nucleic acid analog used to modify gene expression, particularly to knock down gene function. It contains DNA bases attached to a backbone of methylenemorpholine rings linked through phosphorodiamidate groups. PMOs block access of other molecules to small (˜25 base) specific sequences of the base-pairing surface of ribonucleic acid (RNA).

The RNA may be a messenger RNA (mRNA), i.e. a single-stranded molecule of RNA transcribed from genomic DNA or cDNA. The primary transcript of mRNA (pre-mRNA) may still comprise introns, which are removed via splicing into mature mRNA. The mRNA contains the coding nucleic acid sequence translated into an amino acid sequence that forms a protein. The mature mRNA typically contains a Cap structure and a 5′ untranslated region (5′UTR) at the 5′end of the coding sequence and a 3′ untranslated region (3′UTR) and a poly-A tail at the 3′ end. The mRNA may contain regulatory elements, such as microRNA binding sequences in the 3′UTR for tissue-specific protein translation. An mRNA can be monocistronic (comprising the coding sequence for a single polypeptide) or polycistronic (comprising the coding sequence for more than one polypeptide). The mRNA may encode any protein of interest, such as an antigen (for use in a vaccine), a cytokine or other immunostimulatory protein, an antitumoral protein, a Cas protein, an enzyme and the like. The antigen may be, e.g., an antigen from a pathogen (e.g., a virus derived, a bacterium derived, a yeast derived or a parasite derived antigen), a tumor antigen (a tumor specific antigen including a neoantigen or a tumor associated antigen). Neoantigens (also referred to as neoepitopes) are tumor-specific antigens generated by mutations in tumor cells (somatic mutations), which are typically recognized by autologous T cells in the host. Neoantigens are not subject to central immune tolerance and are not expressed in healthy tissues and are therefore attractive targets for therapeutic cancer vaccines. Following identification of neoantigens in tumor samples from individual patients, the epitopes of the neoantigens recognized by autologous T cells are typically expressed as a string comprising several epitopes of the same or different neoantigens as mRNA (or as a non-viral DNA expression vector, such as pDNA) and may be delivered as personalized medicine using the carriers and nanoparticles of the present invention. For mRNA delivery for protein expression (e.g., antigen expression) it is sufficient that the cargo is delivered to the cytosol. The single-stranded RNA can be a replicon, preferably self-replicating or self-amplifying RNA. The replicon can be replicated by a replicase from an alphavirus.

The RNA may further be an siRNA, miRNA or any other mediator of RNA interference (RNAi). RNAi is a biological process in which double stranded RNA molecules are involved in sequence-specific suppression of gene expression (gene silencing) by double-stranded RNA through specific mRNA cleavage or translational repression. MicroRNAs (miRNAs) are natural occurring double stranded non-coding RNAs that function in RNA silencing and post-transcriptional regulation of gene expression of several genes in parallel. By contrast, siRNAs are typically highly target specific. These molecules are shorter (commonly 21-13 bp) compared to mRNA and need to be delivered to the cytosol. RNA interference may also be mediated by small hairpin RNA (shRNA), an artificial RNA molecule with tight hairpin turn. Expression of shRNA in cells is typically accomplished by delivery of a plasmid, rather than RNA. The RNA may further be double stranded polyinosinic-polycytidylic acid, poly(I:C) which is immunostimulatory and cytotoxic by stimulating innate immune reaction (see e.g., D. Schaffert et al, Pharm Res 2011, 28, 731-741).

The cargo can also be an antisense oligonucleotide, i.e., a single-strand DNA or RNA complementary to a protein-coding mRNA sequence.

Plasmid DNA (pDNA) encompasses any plasmid known in the art. Typically, a plasmid comprises an expression cassette comprising a promoter, an open reading frame and a termination sequence. The expression cassette may encode an RNA, such as an mRNA (and further a protein) or an RNAi mediator (e.g., a small hairpin RNA (shRNA)). The plasmid may further comprise a selectable marker and/or an origin of replication. Since plasmids are DNA molecules that need to be transcribed into RNA, i.e., mRNA for protein expression, it needs to be delivered to the cell nucleus. Other non-viral DNA expression vectors are also a suitable cargo and may be used instead of pDNA, such as a plasmid-derived minicircle DNA, a dumbbell-shaped DNA (dbDNA, doggybone DNA), a cosmid, a bacterial artificial chromosome (BAC), a yeast artificial chromosome (YAC) or a mammalian artificial chromosome (MAC). The non-viral DNA expression vectors pDNA, dbDNA, minicircles and BACs are particularly suitable as non-viral DNA expression vector cargo in the context of the present invention.

Cas proteins are RNA-directed nucleases, wherein a small guide RNA (gRNA) complementary to a target site guides the nuclease to this site. For example, Cas9 or Cas12 introduces double-strand breaks (DSB) at targeted DNA loci (Cas9 blunt end, Cas12 staggered ends). Together with the guide RNA the Cas protein forms the ribonucleoprotein (RNP). The Cas protein may be delivered as protein (Cas protein/gRNA ribonucleoprotein (RNP)) or as mRNA encoding the Cas protein (Cas mRNA/gRNA). The nanoparticle may further comprise a donor DNA for HDR. The donor DNA may be provided as plasmid DNA, as linear double-stranded DNA or as single-stranded DNA.

The guide RNA (gRNA) is complementary to a target DNA locus and guides the Cas protein endonuclease to this site. The gRNA may be a CRISPR RNA (crRNA), a crRNA that pairs with trans-activating crRNAs (tracrRNA), an artificial single-guide RNA (sgRNA), an artificial prime editing guide RNA (pegRNA), a chimeric single-guide RNA (cgRNA) or other RNA molecules which form a complex with a Cas protein and guide it to the target DNA sequence. Preferably, the guide RNA is a single-guide RNA (sgRNA), an artificial RNA consisting of tracr RNA, crRNA and an artificial RNA linker. The single-guide RNA may also be a modified and/or improved sgRNAs, such as tru-gRNA (using a spacer sequence with <20 nucleotides complementary to the protospacer target) and hp-sgRNA (comprising an extension on the 5′end of the spacer). However, the person skilled in the art would know (see, e.g., Anzalone et al., Nature Biotechnology, 2020, 38: 824-844) that the type of guide RNA may depend on the intended use and on the Cas protein used. While Cas9 proteins typically used in complex with an sgRNA, many Cas12 nucleases are guided by a single crRNA. Cas12 nucleases possess just a single RuvC-like nuclease domain that mediates targeted DNA cleavage of both strands, typically staggered cuts within the region of the protospacer.

The Cas protein may be any CRISPR-associated endonuclease, preferably class 2 (types II, V and VI) proteins, which have single-subunit effectors. Suitable Cas proteins include, without being limited thereto, type II Cas proteins, e.g., Cas9 (such as SpCas9, SaCas9, CjCas9, StCas9 or NmeCas9); type V Cas proteins, e.g., Cas12, including without being limited thereto Cas12a, Cas12f, Cas12b, Cas12i, Cas12e and Cas12g; and type VI Cas proteins, e.g., Cas13, including without being limited thereto Cas13a, Cas13b, Cas13c and Cas13d, including engineered variants thereof (engineered Cas variants). In certain embodiments the Cas protein is selected from the group consisting of Cas9 protein, Cas 12 protein, Cas 13 protein, and engineered variants thereof, preferably the Cas protein is a Cas9 protein, a Cas 12 protein or an engineered variant thereof, more preferably the Cas protein is a Cas9 protein or an engineered variant thereof, such as a base editor or a prime editor.

Engineered Cas variants include, without being limited thereto, mutant and/or fusion proteins, such as variants with altered PAM compatibilities, such as less restrictive or different PAM compatibility of Cas9 or Cas12 variants; variants with higher DNA specificity, such as variants with reduced off-target Cas nuclease activity (e.g., eSpCas(1.1), SpCas9-HF1, HypaCas9, evoCas9, Sniper-Cas9, HiFiCas9, enAsCas12a-HF1); engineered Cas-domain-fused transposase and recombinase systems; base editors and prime editors.

Commonly used exemplary engineered variants are, for example, Cas nickase (e.g., nickase Cas9n) comprising an inactivated nuclease (e.g., HNH) and a nuclease-deficient dCas (e.g., dCas9), with impaired or no nuclease activity.

Cas proteins may be derived from different species, such as Streptococcus pyogenes, Staphylococcus aureus, Campylobacter jejuni, Streptococcus thermophilus or Neisseria meningitidis. For example, Cas9 orthologs include, without being limited thereto, Streptococcus pyogenes Cas9 (SpCas9), Staphylococcus aureus Cas9 (SaCas9), Campylobacter jejuni Cas9 (CjCas9), Streptococcus thermophilus Cas9 (StCas9), and Neisseria meningitidis Cas9 (NmeCas9) from. Cas orthologs may differ in the recognized PAM sequences and in size. The most often used Cas9 protein is SpCas9.

In certain embodiments the Cas protein is a Cas9, a Cas12, a Cas13 protein or an engineered variant thereof (also referred to as derivative thereof). In some embodiments the Cas protein is a base editor or a prime editor, preferably a Cas9 base editor or a Cas9 prime editor.

The Cas protein forms the Cas protein/gRNA complex, thus in certain embodiments the one or more Cas protein/gRNA RNP complex(es) is/are Cas9/gRNA RNP complex(es), preferably Cas9/sgRNA RNP complex(es), optionally further comprising a donor DNA. This includes Cas9 nucleases as well as fusion proteins thereof, such as base editors and prime editors.

Since this system is modular it can be readily engineered. One of two cellular mechanisms then repairs the cut DNA by non-homologous end-joining (NHEJ), which introduces insertions or deletions (indels) or by homology-directed repair (HDR), which uses a donor DNA as template to introduce specific modifications near the target site. Thus, for some applications delivery of just the DNA nuclease or an mRNA encoding the DNA nuclease is sufficient. For example, the Cas protein/gRNA RNP complex can be used to knockout alleles that underlie autosomal dominant genetic disorders, such as Huntington's disease and amyotrophic lateral sclerosis or for exon skipping or removal of a cryptic splice site, such as for Duchenne's muscular dystrophy and Leber's congenital amaurosis type 10, respectively. Moreover, base editors may be used to edit point-mutations in disease-causing alleles and the more recently developed prime editors may be used to correct not only point mutations, but also small indels without the induction of a double-stranded break. In addition to base editors and prime editors, genome editing may be affected using nuclease-mediated, double-stranded break to trigger HDR via the co-delivery of a donor DNA.

In polyplexes for DNA and RNA cargo (e.g., non-viral DNA expression vectors, such as pDNA, or RNA such as mRNA or siRNA) U-shape structures based on LAF 12Oc are preferred and U1, U3 and U4 seem to be slightly preferred over U2 structures. For bulky carriers with B2 structures, shorter LAFs such as 8Oc or 12Bu are preferred over 12Oc. In general, a PCD:ACD ratio of 1:4 was less suitable for nanoparticle formation, especially for non-viral DNA expression vector, such as pDNA, and mRNA polyplexes. This was the case for all investigated topologies, i.e., combs (1616), bundles (1613) and U-shapes (U1—1 718; U2—1720; U3—1612; U4—1716). For many LAF carriers, an N/P ratio of 6 was not enough to sufficiently form defined polyplexes. However, increasing the N/P ratio often led to homogenous particle formation with N/P 12 and 18 turning out to be most suitable for most of the LAF carriers. U-Shapes with one PCD (of the Stp1-series) needed higher N/P ratios than those with two PCDs (2 Stp units) to be able to form stable polyplexes.

More specifically, for DNA (e.g. pDNA) as cargo in polyplexes, B1 and B2 structures are effective. For B1 structures, 2 PCD are preferred over 1, preferably the ratio PCD:ACD is 1:1 (with a PCD:ACD content of 2:2). This refers to longer ACDs (i.e., C20). For B2 structures, the ratio PCD:ACD of 1:2 (preferably with a PCD:ACD content of 2:4) and 1:4 are preferred, more preferably a ratio of 1:2 with a PCD:ACD content of 2:4. The B2 structures are particularly effective for shorter ACDs (e.g., C16, such as 8Oc or 12Bu). U-shape structures are most preferred, preferably U1, U3 or U4 structures with a ratio of 1:2 (with a PCD:ACD content of 1:2 or 2:4), more preferably U1 structures with a ratio of 1:2 (with a PCD:ACD content of 1:2 or 2:4), preferably with a PCD:ACD content 1:2. In the ACD x is 4-12, preferably 4-10, more preferably 4-8 and R1 and R2 are C6-16, preferably C8-C12.

For longer single-stranded RNA (e.g., mRNA) as cargo in polyplexes, B2 structures are more effective compared to B1 structures and 1 PCD is preferred over 2, preferably the ratio PCD:ACD is 1:4. B2 bundles with short LAFs such as 8Oc and 12Bu are most effective mRNA carriers. U-shape structures are most preferred for longer LAFs such as 12Oc, preferably U1, U3 or U4 structures with a ratio of 1:2 (e.g., with a PCD:ACD content of 1:2 or 2:4), more preferably U1 structures with a PCD:ACD content of 1:2 or 2:4, preferably 1:2. In the ACD x is 4-12, preferably 4-10, more preferably 4-8 and R1 and R2 are C6-16, preferably C8-C12.

For mixed DNA and RNA (e.g., mRNA/pDNA) as cargo in polyplexes, U-shape structures are most preferred, preferably U1 or U4 structures with a PCD:ACD ratio of 1:2 (e.g., with a PCD:ACD content of 1:2 or 2:4) for U1 and 1:4 for U4, more preferably U1 structures with a ratio of 1:2 (e.g., with a PCD:ACD content of 1:2 or 2:4), preferably a PCD:ACD content of 1:2. In the ACD x is 4-12, preferably 4-10, more preferably 4-8 and R1 and R2 are C6-16, preferably C8-C12.

For short RNAs (e.g., siRNA, miRNA) as cargo in polyplexes, U-shape structures are most preferred, preferably U1 or U4 structures with a PCD:ACD ratio of 1:2 or 1:4 (e.g., with a PCD:ACD content of 1:2, 1:4 or 2:4) for U1 and 1:2 or 1:4 (e.g., with a PCD:ACD content of 1:4 or 2:4) for U4, more preferably U4 structures with a ratio of 1:4 (e.g., with a PCD:ACD content of 1:4). In the ACD x is 4-12, preferably 4-10, more preferably 4-8 and R1 and R2 are C6-16, preferably C8-C12.

For polyplexes comprising Cas mRNA/gRNA (e.g., Cas mRNA/gRNA or Cas mRNA/gRNA/ssDNA or more specifically Cas mRNA/sgRNA or Cas mRNA/sgRNA/ssDNA) as cargo, B2 structures are more effective compared to B1 structures and 1 PCD is preferred over 2, preferably the ratio PCD:ACD is 1:4, more preferably the PCD:ACD content is 1:4. U-shape structures are most preferred, preferably U1, U3 or U4 structures with a PCD:ACD ratio of 1:2 (e.g., with a PCD:ACD content of 1:2 or 2:4), more preferably U1 structures with a ratio of 1:2 (e.g., with a PCD:ACD content of 1:2 or 2:4), preferably a PCD:ACD content of 1:2 or U3 or U4 structures with a PCD:ACD content of 2:4. In the ACD x is 4-12, preferably 4-10, more preferably 4-8 and R1 and R2 are C6-16, preferably C8-C12.

For mRNA LNPs, B2 and U-shape structures are preferred. For B2 structures 1 PCD is preferred over 2, preferably the ratio PCD:ACD is 1:4 (e.g., with a PCD:ACD content of 1:4). For U-shape structures U2 and U4 structures are preferred, preferably with a PCD:ACD ratio of 1:4. In the ACD x is 4-12, preferably 4-10, more preferably 4-8 and R1 and R2 are C6-16, preferably C8-C14.

For siRNA LNPs, B2 and U-shape structures are preferred and B2 structures seem to be most advantageous. For B2 structures 1 PCD is preferred over 2, preferably with a PCD:ACD ratio of 1:4. In the ACD x is 4-12, preferably 4-10, more preferably 4-8 and R1 and R2 are C6-16, preferably C8-C16.

In one aspect, the nanoparticle of the invention is used in therapy. Depending on the cargo, a variety of diseases can be treated, including, without being limited thereto, a disease selected from the group consisting of cancer, a genetic disease, an infectious disease, a cardiovascular disease, a metabolic disease, a neurodegenerative or neuromuscular disease, a hematological disease, a hereditary eye disease and an autoimmune disease.

In another aspect, the nanoparticle of the invention is used in treating cancer, a genetic disease, an infectious disease, a cardiovascular disease, a metabolic disease, a neurodegenerative or neuromuscular disease, a hematological disease, a hereditary eye disease or an autoimmune disease.

The nanoparticle according to the invention may be used in vivo or ex vivo for cargo delivery. Thus, the nanoparticle is delivered to a cell of the subject to be treated in vivo or ex vivo. For therapeutic uses ex vivo nanoparticle delivery to cells of the subject to be treated (autologous cells) or donor cells (allogenic cells) is followed by adoptive cell transfer of said cells to a subject. Prior to adoptive cell transfer the cell may be screened for successful cargo delivery, product of interest expression (nucleic acid, RNA and/or protein) and/or effect of cargo delivery, e.g., protein expression, gene silencing, genome editing etc. Routes for adoptive transfer of the genome edited cells to the subject are known in the art and include, without being limited thereto, intravenous administration, subcutaneous administration and intramuscular administration, particularly intravenous administration.

In vivo delivery involves local administration or systemic administration and may require the coupling of a targeting ligand. Alternatively, targeted delivery by be achieved for cell types such as macrophages, with a high degree of phagocytosis and endocytosis. The nanoparticle may therefore be administered by any route, including, without being limited thereto intravenous administration, subcutaneous administration, intradermal administration, intramuscular administration, oral administration, intranasal administration, inhalation, vaginal administration, intravitreal administration, or intrathecal administration.

In yet another aspect the invention relates to a pharmaceutical composition comprising a nanoparticle and at least one pharmaceutically acceptable excipient. The pharmaceutical composition comprising the nanoparticle according to the invention may be cryo-conserved, lyophilized or in an isotonic solution (i.e., in a physiological buffer).

In addition to in vivo or ex vivo delivery, the nanoparticle according to the invention may further be used for in vitro cargo delivery, i.e., for non-therapeutic purposes.

Thus, in another aspect the invention relates to an in vitro method for introducing a nucleic acid and/or protein into mammalian cells comprising contacting a mammalian cell in vitro with the nanoparticle according to the invention comprising a nucleic acid and/or protein. The nucleic acid may be RNA or DNA, preferably mRNA, Cas mRNA/gRNA, siRNA, miRNA, poly(I:C), phosphodiamidate-morpholino-oligomer (PMO), non-viral DNA expression vector (e.g., pDNA), or Cas protein/gRNA ribonucleoprotein (RNP). Any mammalian cell may be suitable in the context of the present invention including cell lines and primary cells, suspension and adherent cells or even organoids. For example, the mammalian cells may be human or rodent cells, including, without being limited thereto CHO cells BHK cells, HEK293 cells, HeLa cells, HepG2 cells and derivatives thereof. The person skilled in the art will understand that derivatives of CHO cells include, e.g., glutathione deficient CHO cells such as CHO-K1 cells and the like. Moreover, derivatives of HEK 293 cells include, e.g., HEK293T, HEK293E, HEK293F, HEK293SF cells and the like.

In yet another aspect the invention relates to a use of the carrier according to the invention or the nanoparticle according to the invention for cellular delivery of a cargo. Preferably the cargo comprises or is a nucleic acid and/or a protein, more preferably a nucleic acid. The uses of the invention are in vitro uses, i.e., for non-therapeutic purpose. In certain embodiments the target cell is a mammalian cell, preferably as specified herein above.

### EXAMPLES

### I. Methods

### 1. Synthesis of Aldehydes

1.1 Synthesis of Tetradecanal (C₁₄H₂₈O)

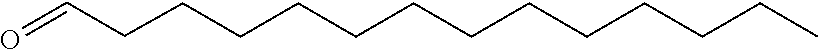

10 mL dry dichloromethane (DCM) and 5 mL oxalyl chloride (55 mmol, 2.2 eq.) were added to a 200 mL round bottom flask equipped with a septum under nitrogen atmosphere at −10° C. 8.5 mL dimethyl sulfoxide (DMSO; 120 mmol, 4.8 eq.) dissolved in 25 mL dry DCM were added to the solution with a syringe over 2 min. Afterwards, 5.36 g tetradecan-1-ol (25 mmol, 1 eq.) dissolved in 25 mL dry DCM were added and the mixture was stirred for 5 min at −10° C. In a next step, 17.4 mL trimethylamine (125 mmol, 5 eq.) were added to the solution, followed by 15 min of stirring at −10° C. The reaction was warmed up to RT prior to the addition of 50 mL H₂O. The reaction was extracted 3× with DCM (50 mL each) and washed with 50 mL brine, 1% (v/v) hydrochloric acid (HCl), H₂O and 5% (v/v) sodium carbonate (Na₂CO₃). The combined organic phases were dried over anhydrous magnesium sulfate (MgSO₄), filtrated with a Buchner funnel, and evaporated to provide tetradecanal. The product was confirmed by EI-MS (electron ionization mass spectrometry) and 1H-NMR (nuclear magnetic resonance) spectroscopy.

Yield: 92%; white solid; MW [Da]: 212.38

EI-MS: 196.22 (C₁₄H₂₂·), 155.19 (C₁₀H₁₉·), 127.18 (C₈H₁₅O·), 85.15 (C₆H₁₃·), 57.15 (C₄H₉·).

¹H-NMR (400 MHz, CDCl₃) δ (ppm)=0.77-0.83 (m, 3H, —CH₃), 1.14-1.27 (m, 20H, —CH₂—), 1.40-1.57 (m, 2H, —CH₂—), 2.28-2.335 (m, 2H, —CH₂—CO).

1.2 Synthesis of Hexadecanal (C₁₆H₃₂O)

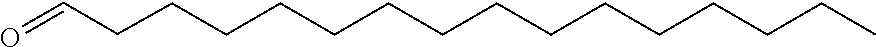 [SMILES: CCCCCCCCCCCCCCCC=O]

2 g hexadecan-1-ol (8.26 mmol, 1 eq.) were added to a 100 mL round bottom flask and dissolved in 20 mL dry DCM. After flushing the flask with nitrogen, 5.253 g Dess-Martin-periodinane (DMP, 12.39 mmol, 1.5 eq.) were added. The mixture was stirred at 0° C. for 3 h. The reaction was quenched by addition of 12 mL saturated sodium sulfite (Na₂SO₃) and 20 mL sodium bicarbonate (NaHCO₃) solution and stirring for 5 min. Afterwards, the mixture was warmed up to RT and extracted 3× with DCM (25 mL each). The combined organic phases were dried over anhydrous Na₂SO₄ and filtrated with a Buchner funnel. Before purifying the crude product, the volume was concentrated to 5 mL by evaporating under reduced pressure. Purification was performed by silica gel chromatography (petrolether:ethylacetate 15:1 (v/v)). The product was confirmed by EI-MS, ¹H- and ¹³C-NMR spectroscopy.

Yield: 95%; white solid; MW [Da]: 240.43

EI-MS: 222.20 (C₁₆H₃₂·), 196.20 (C₁₄H₂₉·), 111.17 (C₇H₁₃O·), 83.16 (C₆H₁₃·), 57.15 (C₄H₉·).

¹H-NMR (400 MHz, CDCl₃) δ (ppm)=0.875-0.925 (m, 3H, —CH₃), 1.24-1.37 (m, 24H, —CH₂—), 1.58-1.69 (m, 2H, —CH₂—), 2.41-2.47 (m, 2H, —CH₂—CO).

¹³C-NMR (400 MHz, CDCl₃) δ (ppm)=14.32 (m, CH₂, —CH₃), 43.93 (s, 1C, —CH₂—CO), 203 (s, 1C, C═O).

2. Synthesis of lipo amino fatty acids (LAFs)

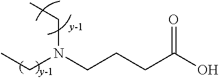

y=12 (12Bu), 16 (16Bu)

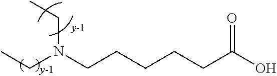

with y=12 (12He), 14 (14He)

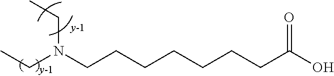

with y=8 (8Oc), 10 (10Oc), 12 (12Oc)

200 mg of indicated amino fatty acid (1 eq.; 4-aminobutyric acid, δ-aminohexanoic acid, or 8-aminooctanoic acid) were dissolved in 25 mL methanol (MeOH), added to a 100 mL round bottom flask, and stirred for 15 min at RT. Afterwards, 2.5 eq. of indicated fatty aldehyde (octanal, decanal, dodecanal, tetradecanal, or hexadecanal; the latter two pre-dissolved in 5 mL THF), 2.5 eq. of NaBH3CN and 0.8 eq. of acetic acid were added. The mixture was stirred for 24 h at RT and monitored by thin-layer chromatography using DCM/MeOH 9:1 (v/v) as mobile phase. Consumption of educts was detected by using basic potassium permanganate (KMnO4) solution. After 24 h, 1 eq. of fatty aldehyde and 1 eq. of NaBH3CN were added, and the reaction was conducted for additional 24 h (8Oc, 10Oc, 12Oc, 12Bu, 12He) or 48 h (14He, 16Bu). The solvent was then evaporated under reduced pressure. To remove excess reducing agent, the dry mixture was redissolved in pure DCM and filtered. After concentrating the filtrate to 5 mL, the crude product was purified by silica gel chromatography (DCM/MeOH; 10:0 to 15:1 (v/v) for 12Bu, 12He, and 14He; 10:0 to 20:1 (v/v) for 16Bu; 50:1 bis 20:1 (v/v) for 8Oc, 10Oc, and 12Oc). The product was confirmed by ESI (electrospray ionization)-MS and 1H-NMR spectroscopy.

Note: Acetic should be added at last and only in small/catalytic amounts (<1 eq.) to avoid byproducts due to side-reactions such as imine-catalyzed aldol addition.

### Analytical Data of the Synthesized LAFs

12Bu (C₂₈H₅₇NO₂) Yield: 83%; white solid.

ESI-MS: 439.77 Da (calculated), 440.45 Da (found, [M+H]⁺).

¹H-NMR (400 MHz, CDCl₃) δ (ppm)=0.88-0.92 (m, 6H, —CH₃, dodecanal), 1.24-1.35 (m, 40H, —CH₂—), 1.60-1.68 (m, 2H, —CH₂—N—), 1.82-1.89 (m, 2H, —CH₂—CO), 2.60-2.84 (m, 6H, —CH₂—N—).

16Bu (C₃₆H₇₃NO₂) Yield: 85%, white solid.

ESI-MS: 551.99 Da (calculated), 552.57 Da (found, [M+H]⁺).

¹H-NMR (400 MHz, CDCl₃) δ (ppm)=0.87-0.93 (m, 6H, —CH₃, hexadecanal), 1.22-1.37 (m, 56H, —CH₂—), 1.60-1.69 (m, 2H, —CH₂—), 1.83-1.90 (m, 2H, —CH₂—CO), 2.59-2.90 (m, 6H, —CH₂—N—).

12He (C₃₀H₆₁NO₂) Yield: 79%; colorless oil.

ESI-MS: 467.82 Da (calculated), 468.48 Da (found, [M+H]⁺).

¹H-NMR (400 MHz, CDCl₃) δ (ppm)=0.61-0.70 (m, 6H, —CH₃, dodecanal), 0.98-1.18 (m, 44H, —CH₂—), 1.40-1.58 (m, 2H, —CH₂—), 2.12-2.22 (m, 2H, —CH₂—CO), 2.70-2.88 (m, 6H, —CH₂—N—).

14He (C₃₄H₆₉NO₂) Yield: 85%; colorless oil.

ESI-MS: 523.93 Da (calculated), 524.54 Da (found, [M+H]⁺).

¹H-NMR (400 MHz, CDCl₃) δ (ppm)=0.87-0.94 (m, 6H, —CH₃, tetradecanal), 1.21-1.41 (m, 52H, —CH₂—), 1.65-1.77 (m, 2H, —CH₂—), 2.38-2.44 (m, 2H, —CH₂—CO), 2.96-3.05 (m, 6H, —CH₂—N—).

8Oc (C₂₄H₄₉NO₂) Yield: 72%; colorless oil.

ESI-MS: 383.66 Da (calculated), 384.38 Da (found, [M+H]⁺).

¹H-NMR (400 MHz, CDCl₃) δ (ppm)=0.65-0.95 (m, 6H, —CH₃, octanal), 1.15-1.53 (m, 26H, —CH₂—CH₂—), 1.55-1.88 (m, 8H, —CH₂—), 2.24-2.44 (m, 2H, —CH₂—CO—), 2.28-3.12 (m, 6H, —CH₂—N—).

10Oc (C₂₈H₅₇NO₂) Yield: 68%; colorless oil.

ESI-MS: 439.77 Da (calculated), 440.45 Da (found, [M+H]⁺).

¹H-NMR (400 MHz, CDCl₃) δ (ppm)=0.65-0.95 (m, 6H, —CH₃, decanal), 1.15-1.53 (m, 34H, —CH₂—CH₂—), 1.55-1.88 (m, 8H, —CH₂—), 2.24-2.44 (m, 2H, —CH₂—CO—), 2.28-3.12 (m, 6H, —CH₂—N—).

12Oc (C₃₂H₆₅NO₂) Yield: 71%; colorless oil.

ESI-MS: 495.88 Da (calculated), 496.51 Da (found, [M+H]⁺).

¹H-NMR (400 MHz, CDCl₃) δ (ppm)=0.65-0.95 (m, 6H, —CH₃, dodecanal), 1.15-1.53 (m, 42H, —CH₂—CH₂—), 1.55-1.88 (m, 8H, —CH₂—), 2.24-2.44 (m, 2H, —CH₂—CO), 2.28-3.12 (m, 6H, —CH₂—N—).

3. Synthesis of Artificial Amino Acids (Oligo(Alkylamino) Acids, e.g. Stp and Analoga)
3.1 Synthesis of N-(tert-Butoxycarbonyl)iminodiacetic acid (Boc-IDA)

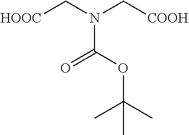 [SMILES: CC(C)(C)OC(=O)N(CC(=O)O)CC(=O)O]

N-tert-Butoxycarbonyl protected iminodiacetic acid (Boc-IDA) was synthesized using the procedure reported by Cookson et al. (Cookson, J., Amide functionalised dithiocarbamate ruthenium(II) bis-bipyridyl receptors: A new class of redox-responsive anion sensor, Inorganica Chimica Acta, 2008, 361, 1689-1698) with slight modification. Briefly 13.3 g iminodiacetic acid (0.1 mol) and 200 mL 1,4-dioxane were put into a 1 L round-bottom flask. 200 mL of 1 M sodium hydroxide solution were added and the mixture was stirred until a clear solution formed. 24.0 g of di-tert-butyl dicarbonate (0.11 mol, 1.1 eq) were dissolved in 50 mL 1,4-dioxane and added to the reaction mixture, which was stirred at RT for 72 h. The mixture was concentrated to approx. 200 mL under reduced pressure, washed twice with 150 mL diethyl ether. After acidification with 100 mL 10% HCl, the reaction product was extracted with EtOAc (3×150 mL). The organic phases were combined and dried over anhydrous sodium sulfate. The EtOAc was evaporated after filtration, yielding 22.3 g of Boc-IDA (0.096 mol, 96%) as white crystals.

3.2 Synthesis of N-(tert-Butoxycarbonyl)iminodiacetic acid anhydride (Boc-IDA anhydride)

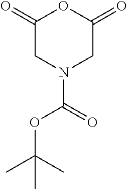

The cyclic anhydride of Boc-IDA was prepared by using dicyclohexylcarbodiimide (DCC) as dehydrating agent. 10.0 g of Boc-IDA (43 mmol) were put into a 500 mL round-bottom flask and 250 mL DCM were added. 8.9 g of DCC (43 mmol, 1 eq) were dissolved in 50 mL DCM and added into the round-bottom flask. The heterogeneous mixture was stirred at RT overnight. The next day the mixture was concentrated to a volume of approx. 100 mL under reduced pressure and the insoluble dicyclohexyl urea was removed by filtration. The DCM was removed in the rotary evaporator and at high vacuum to yield 8.4 g of Boc-IDA anhydride (39 mmol, 91%) as a solid.

### 3.3 Synthesis of Protected Artificial Amino Acid Building Blocks (Depicted in Scheme 1)

The synthesis of protected building blocks Fmoc-Gtt(Boc₂)-OH, Fmoc-Stp(Boc₃)-OH, Fmoc-Gtp(Boc₃)-OH, Fmoc-Ptp(Boc₃)-OH, Fmoc-Htp(Boc₃)-OH, Fmoc-chGtp(Boc₃)-OH, Fmoc-Ntp(Boc₃)-OH, Fmoc-Boc-IDAtp(Boc₃)-OH and Fmoc-dGtp(Boc₃)-OH was carried out with cyclic dicarboxylic acid anhydrides analog to the description published by Schaffert et al. (Schaffert, D. et al., Novel Fmoc-polyamino acids for solid-phase synthesis of defined polyamidoamines, Organic letters, 2011, 13.7: 1586-1589). Briefly, the two primary amines of the polyamines 1 (TETA, TEPA) were selectively protected with ethyl trifluoroacetate (EtOTFA). Subsequently the remaining secondary amines were Boc-protected by reaction with di-tert-butyl dicarbonate (Boc₂O) in a one-pot reaction. Work-up and recrystallization gave the compounds 2, bis-tfa-Tt(Boc₂) or bis-tfa-Tp(Boc₃), respectively. The primary amines were deprotected by alkaline hydrolysis with aqueous NaOH containing 45% EtOH to obtain the compounds 3, Tt(Boc₂) or Tp(Boc₃).

In the final step, the two primary amines were asymmetrically substituted by reaction with a cyclic anhydride (succinic anhydride for Fmoc-Stp(Boc₃)-OH; glutaric anhydride for Fmoc-Gtp(Boc₃)-OH and Fmoc-Gtt(Boc₂)-OH; phthalic anhydride for Fmoc-Ptp(Boc₃)-OH; 3-oxaspiro[5,5]undecan-2,4-dion for Fmoc-chGtp(Boc₃)-OH; 1,2-cyclohexanedicarboxylic anhydride for Fmoc-Htp(Boc₃)-OH; 2,3-napthalic anhydride for Fmoc-Ntp(Boc₃)-OH; Boc-IDA anhydride for Fmoc-Boc-IDAtp(Boc₃)-OH; diglycolic anhydride for Fmoc-dGtp(Boc₃)-OH) and N-(fluorenyl-9-methoxycarbonyloxy)-succinimid (Fmoc-OSu). Purification of the products 4a was carried out by dry column vacuum chromatography (DCVC) (D. S. Pedersen, C. Rosenbohm, Dry Column Vacuum Chromatography Synthesis, (2001) 2431-2434).

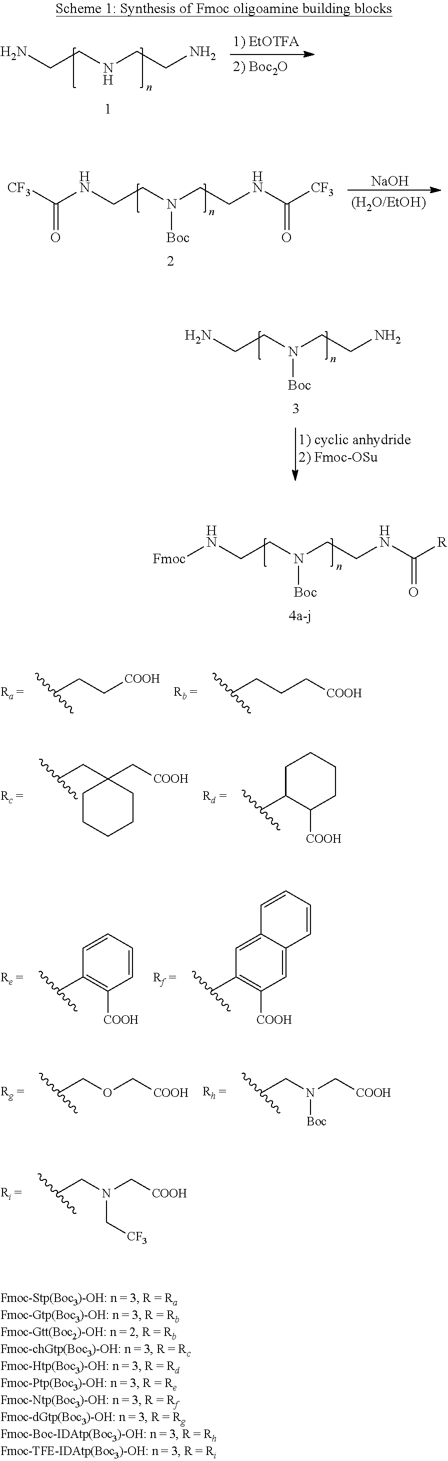

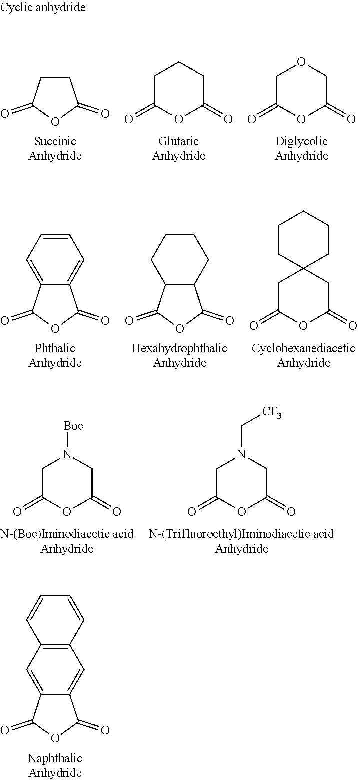

### 3.4 Synthesis of Protected Artificial Amino Acid Building Block Fmoc-GEIPA(Boc₂)-OH (Depicted in Scheme 2)

The protected building block Fmoc-GEIPA(Boc₂)-OH was synthesized by exact adaption of the synthesis protocol described a end using the oligoamine 3,3′-ethylenediiminodipropylamine (EIPA) instead of TETA or TEPA.

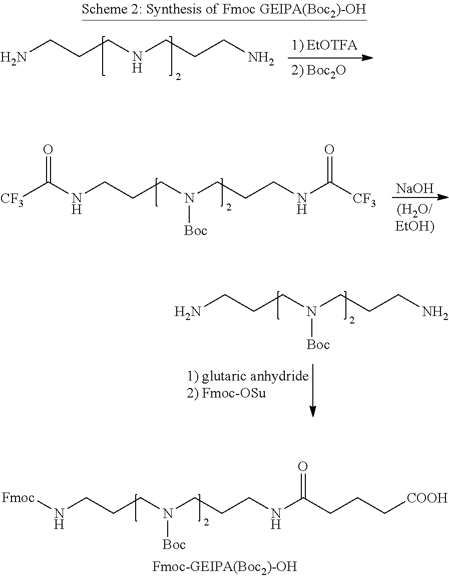

### Analytical Results:

Fmoc-Stp(Boc₃)-OH. Yield: 77%. ¹H NMR (400 MHz, CDCl₃) δ (ppm)=7.78 (d, 2H, ArH-Fmoc), 7.61 (d, 2H, ArH-Fmoc), 7.41 (t, 2H, ArH-Fmoc), 7.31 (t, 2H, ArH-Fmoc), 4.40 (m, 2H, CH₂-Fmoc), 4.21 (m, 1H, CH-Fmoc), 3.47-3.20 (m, 16H, CH₂-Tepa), 2.67 (t, 2H, CH₂CONH-Suc), 2.48 (m, 2H, CH₂COOH-Suc), 1.46 (s, 27H, CH₃-tert-but). ESI-MS: calculated 811.4; found 810.4 [M−H]⁺.

Fmoc-Boc-IDAtp(Boc₃)-OH. Yield: 29%. ¹H NMR (400 MHz, CDCl₃) δ (ppm)=7.77 (d, 2H, ArH-Fmoc), 7.59 (d, 2H, ArH-Fmoc), 7.40 (t, 2H, ArH-Fmoc), 7.31 (t, 2H, ArH-Fmoc), 4.40 (m, 2H, CH₂-Fmoc), 4.20 (m, 1H, CH-Fmoc), 3.91 (s, 4H, CH₂-IDA) 3.48-3.19 (m, 16H, CH₂-Tepa), 1.43 (s, 36H, CH₃-tert-but). ESI-MS: calculated 926.5; found 925.5 [M−H]⁻.

Fmoc-dGtp(Boc₃)-OH. Yield: 73%. ¹H NMR (400 MHz, CDCl₃) δ (ppm)=7.77 (d, 2H, ArH-Fmoc), 7.60 (d, 2H, ArH-Fmoc), 7.42 (t, 2H, ArH-Fmoc), 7.32 (t, 2H, ArH-Fmoc), 4.41 (m, 2H, CH₂-Fmoc), 4.23 (m, 1H, CH-Fmoc), 4.17 (d, 2H, CH₂CONH-diglycolic acid), 4.10 (d, 2H, CH₂COOH-diglycolic acid), 1.45 (s, 27H, CH₃-tert-but). ESI-MS: calculated 827.4; found 826.4 [M−H]⁻.

Fmoc-Gtp(Boc₃)-OH. Yield: 68%. ¹H NMR (400 MHz, CDCl₃) δ (ppm)=7.79 (d, 2H, ArH-Fmoc), 7.60 (d, 2H, ArH-Fmoc), 7.41 (t, 2H, ArH-Fmoc), 7.32 (t, 2H, ArH-Fmoc), 4.41 (m, 2H, CH₂-Fmoc), 4.22 (m, 1H, CH-Fmoc), 3.48-3.24 (m, 16H, CH₂-Tepa), 2.42 (t, 2H, CH₂CONH-Glu), 2.29 (t, 2H, CH₂COOH-Glu), 1.97 (m, 2H, CH₂CH₂CH₂COOH-Glu), 1.47 (s, 27H, CH₃-tert-but). ESI-MS: calculated 825.4; found 824.4 [M−H]⁻.

Fmoc-Htp(Boc₃)-OH. Yield: 55%. ¹H NMR (400 MHz, CDCl₃) δ (ppm)=7.69 (d, 2H, ArH-Fmoc), 7.51 (d, 2H, ArH-Fmoc), 7.32 (t, 2H, ArH-Fmoc), 7.23 (t, 2H, ArH-Fmoc), 4.30 (m, 2H, CH₂-Fmoc), 4.12 (m, 1H, CH-Fmoc), 3.48-3.08 (m, 16H, CH₂— Tepa), 2.50 (d, 1H, CHCONH-Hexahydrophthalic acid), 2.16 (s, 1H, CHCOOH-Hexahydrophthalic acid), 1.89-1.24 (m, 35H, CH₂-Hexahydrophthalic acid, CH₃-tert-but). ESI-MS: calculated 865.5; found 864.5 [M−H]⁻.

Fmoc-chGtp(Boc₃)-OH. Yield: 63%. ¹H NMR (400 MHz, CDCl₃) δ (ppm)=7.69 (d, 2H, ArH-Fmoc), 7.52 (d, 2H, ArH-Fmoc), 7.33 (t, 2H, ArH-Fmoc), 7.24 (t, 2H, ArH-Fmoc), 4.31 (m, 2H, CH₂-Fmoc), 4.14 (m, 1H, CH-Fmoc), 3.47-3.10 (m, 16H, CH₂-Tepa), 2.46 (s, 2H, CH₂CONH-Cyclohexanediacetic acid), 2.26 (m, 2H, CH₂COOH-Cyclohexanediacetic acid), 1.49-1.21 (m, 37H, CH₂— Cyclohexanediacetic acid, CH₃-tert-but). ESI-MS: calculated 893.5; found 892.5 [M−H]⁻.

Fmoc-TFE-IDAtp(Boc₃)-OH. Yield: 28%. ¹H NMR (400 MHz, CDCl₃) δ (ppm)=8.12-7.24 (m, 8H, ArH-Fmoc), 4.36 (m, 2H, CH₂-Fmoc), 4.15 (t, 1H, CH-Fmoc), 3.71-3.02 (m, 22H, CH2-Tepa, CH2-TFE-IDA), 1.52-1.27 (m, 27H, CH3-tert-but). ESI-MS: calculated 908.4; found 907.4 [M−H]⁻.

Fmoc-Gtt(Boc₂)-OH. Yield: 66%. ¹H NMR (400 MHz, CDCl₃) δ (ppm)=7.79 (d, 2H, ArH-Fmoc), 7.61 (d, 2H, ArH-Fmoc), 7.42 (t, 2H, ArH-Fmoc), 7.32 (t, 2H, ArH-Fmoc), 4.42 (m, 2H, CH₂-Fmoc), 4.22 (m, 1H, CH-Fmoc), 3.52-3.21 (m, 12H, CH₂— Teta), 2.41 (t, 2H, CH₂CONH-Glu), 2.28 (t, 2H, CH₂COOH-Glu), 1.98 (m, 2H, CH₂CH₂CH₂COOH-Glu), 1.45 (s, 18H, CH₃-tert-but). ESI-MS: calculated 682.4; found 681.4 [M−H]⁻.

Fmoc-GEIPA(Boc₂)-OH. Yield: 72%. ¹H NMR (400 MHz, CDCl₃) δ (ppm)=7.78 (d, 2H, ArH-Fmoc), 7.62 (d, 2H, ArH-Fmoc), 7.41 (t, 2H, ArH-Fmoc), 7.32 (t, 2H, ArH-Fmoc), 4.42 (m, 2H, CH₂-Fmoc), 4.23 (m, 1H, CH-Fmoc), 3.43-3.03 (m, 12H, CH₂CH₂CH₂-EIPA, CH₂CH₂-EIPA), 2.44 (t, 2H, CH₂CONH-Glu), 2.30 (t, 2H, CH₂COOH-Glu), 1.99 (m, 2H, CH₂CH₂CH₂COOH-Glu), 1.71 (m, 4H, CH₂CH₂CH₂-EIPA), 1.47 (s, 18H, CH₃-tert-but). ESI-MS: calculated 710.4; found 709.4 [M−H]⁻.

Fmoc-Ptp(Boc₃)-OH. Yield: 45%. ¹H NMR (400 MHz, CDCl₃) δ (ppm)=7.81-7.25 (m, 12H, ArH-Fmoc, ArH-Phthalic acid), 4.41 (m, 2H, CH₂-Fmoc), 4.22 (m, 1H, CH-Fmoc), 3.69-3.17 (m, 16H, CH₂-Tepa), 1.44 (s, 27H, CH₃-tert-but). ESI-MS: calculated 859.4; found 858.4 [M−H]⁻.

Fmoc-Ntp(Boc₃)-OH. Yield: 52%. ¹H NMR (400 MHz, CDCl₃) δ (ppm)=7.98-7.17 (m, 14H, ArH-Fmoc, ArH-Naphthalenedicarboxylic acid), 4.29 (m, 2H, CH₂-Fmoc), 4.12 (m, 1H, CH-Fmoc), 3.62-3.12 (m, 16H, CH₂-Tepa), 1.34 (s, 27H, CH₃-tert-but). ESI-MS: calculated 909.4; found 908.4 [M−H]⁻.

Hydroxystearic acid (OHSteA). Yield: 84%. ¹H NMR (400 MHz, CDCl₃) δ (ppm)=3.57-3.48 (q, 1H, CH—OH), 2.35-2.21 (t, 2H, CH₂COOH), 1.63-1.49 (q, 2H, CH₂CH₂COOH), 1.44-1.11 (m, 26H, —CH₂—), 0.87-0.72 (t, 3H, CH₃). ESI-MS: calculated 300.3; found 299.3 [M−H]⁻.

### 3.5 Synthesis of Different Protected Artificial Amino Acid Building Blocks.

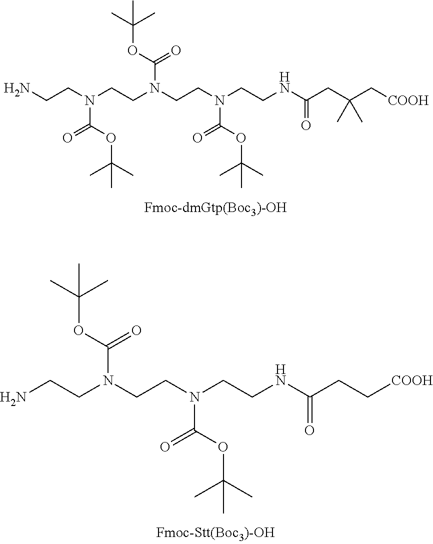 [SMILES: CC(C)(CC(=O)O)CC(=O)NCCN(CCN(CCN(CCN)C(=O)OC(C)(C)C)C(=O)OC(C)(C)C)C(=O)OC(C)(C)C ; CC(C)(C)OC(=O)N(CCN)CCN(CCNC(=O)CCC(=O)O)C(=O)OC(C)(C)C]

The novel artificial amino building blocks were synthesized via the synthetic route described in section 3.3. Starting from Tt(Boc₂) or Tp(Boc₃), the two primary amines were asymmetrically substituted by reaction with a cyclic anhydride (succinic anhydride for Fmoc-Stt(Boc₂)-OH; 4,4-dimethyldihydro-2H-pyran-2,6(3H)-dione for Fmoc-dmGtp(Boc₃)-OH) and Fmoc-OSu.

### Analytical Results:

Fmoc-dmGtp(Boc₃)-OH. Yield: 56%. ¹H NMR (400 MHz, CDCl₃) δ (ppm)=7.75 (d, 2H, ArH-Fmoc), 7.58 (d, 2H, ArH-Fmoc), 7.39 (t, 2H, ArH-Fmoc), 7.30 (t, 2H, ArH-Fmoc), 4.50-4.28 (d, 2H, CH₂-Fmoc), 4.19 (t, 1H, CH-Fmoc), 3.53-3.17 (m, 16H, CH₂-Tepa), 2.45-2.19 (m, 4H, CH₂-dimethylglutaric acid), 1.45 (s, 27H, CH₃-tert-but), 1.07 (s, 6H, CH₃-dimethylglutaric acid). ESI-MS: calculated 853.5; found 854.5 [M−H]⁻.

Fmoc-Stt(Boc₂)-OH. Yield: 56%. ¹H NMR (400 MHz, CDCl₃) δ (ppm)=7.75 (d, 2H, ArH-Fmoc), 7.58 (d, 2H, ArH-Fmoc), 7.39 (t, 2H, ArH-Fmoc), 7.30 (t, 2H, ArH-Fmoc), 4.48-4.30 (m, 2H, CH₂-Fmoc), 4.22 (t, 1H, CH-Fmoc), 3.50-3.3.13 (m, 12H, CH₂-Teta), 2.75-2.32 (m, 4H, CH₂-Suc), 1.45 (s, 18H, CH₃-tert-but). ESI-MS: calculated 668.3; found 667.3 [M−H]⁻.

4. Synthesis of LAF Containing Carriers with Different Topologies Via Solid-Phase Assisted Peptide Synthesis (SPPS)
4.1 Loading of a 2-Chlorotrityl Chloride Resin with an Fmoc-Protected Amino Acid

The desired amount of 2-chlorotrityl chloride resin (1.56 mmol/g chloride) was weighed in a syringe reactor and swelled in anhydrous DCM for 20 min. After swelling, the first Fmoc-protected amino acid of the respective topology (Fmoc-Stp(Boc)₃-OH [Schaffert et al. Org Lett 2011] for comb, bundle and T-shape structures; Fmoc-L-Lys (Dde)-OH for U-shape structures) as well as a 3-fold molar excess of N,N-diisopropylethylamine (DIPEA) were dissolved in anhydrous DCM and added to the resin. The resin was incubated at RT for 75 min. The reaction solvent was drained, and the resin was incubated with a capping solution containing DCM/MeOH/DIPEA (4 mL/3 mL/500 μL per 1000 mg resin) for 60 min to cap residual reactive functions on the resin. After removing the mixture, the resin was washed with 3× dimethylformamide (DMF) and 3×DCM and dried under vacuum.

The resin loading was determined by quantification of released fluorenyl derivatives after piperidine deprotection. Therefore, three samples with an exact amount of resin were treated with 1 mL of 20% (v/v) piperidine in DMF and incubated for 75 min. Afterwards, the samples were vortexed, and 25 μL of each was diluted with 975 μL of DMF. The absorption was measured at 301 nm against 20% (v/v) piperidine in DMF as blank. Finally, the resin loading was calculated according to the following equation: resin load [mmol g⁻¹]=(A×1000)×(m [mg]×7800×df)⁻¹ with df as dilution factor.

The remaining resin was treated 3× with 20% (v/v) piperidine in DMF for 10 min each to remove Fmoc protection groups. The reaction progress was monitored by the Kaiser test. The resin was then washed 3× with DMF and DCM, dried in vacuo and stored at 4° C.

### 4.2 Synthesis of LAF Containing Carriers of Various Topologies Via SPPS

LAF containing carriers were synthesized under standard Fmoc-based SPPS with a 2-chlorotrityl chloride resin as solid support, which was pre-loaded with the first C-terminal amino acid in deprotected form, i.e., with free accessible amino group (for resin loading, see above). The pre-loaded resin was swollen for 20 min in DCM prior to the first coupling step. In general, manual coupling steps were carried out by solving 4 eq. of Fmoc-protected amino acid, and 8 eq. DIPEA in the smallest possible amount of DCM as well as 4 eq. benzotriazol-1-yloxytripyrrolidinophosphonium hexafluorophosphate (PyBOP)/hydroxybenzotriazole (HOBt) in DMF. The reaction mixtures were transferred to the reactor and incubated for 75 min. Fmoc-deprotection was performed 4× with 20% (v/v) piperidine in DMF for 10 min each. After each coupling and deprotection, a washing step (3×DMF, 3×DCM) as well as a Kaiser Test were performed to confirm the reaction completion. The ^{ε}K-^{ε}K-6-Ahx motif for Stp replacement was synthesized by coupling Fmoc-δ-aminohexanoic acid, followed by 2× coupling of Boc-Lys(Fmoc)-OH. Epsilon-poly-lysine motifs ((^{ε}K)ₙ₊₁; wherein n=2-4) are synthesized analogously. Symmetrical branching points were introduced using Fmoc-L-Lys(Fmoc)-OH, and asymmetric branching points were introduced using Fmoc-L-Lys(Dde)-OH. Removal of the Dde [N-(1-(4,4-dimethyl-2,6-dioxocyclohexylidene)ethyl)] protection group was performed with 2% (v/v) hydrazine in DMF for 15 cycles of 2 min each. Then, the resin was washed 5× with 10% (v/v) DIPEA in DMF, followed by 3×DCM and 3×DMF. Prior to Dde deprotection, the N-terminal Fmoc-deprotected amino group was protected by incubating with 10 eq. di-tert-butyl-dicarbonate (Boc anhydride) and DIPEA dissolved in DMF for 75 min. Coupling of LAFs was carried out by dissolving LAFs/DIPEA in DCM, PyBOP/HOBt in DMF and incubating the resin with these solutions under constant shaking for 24 h. Equivalents were calculated relatively to free resin-bound amines after Dde-/Fmoc-deprotection. The negative control motif DodOc was incorporated instead of the LAFs by coupling of Fmoc-8-aminooctanoic acid followed by dodecanoic acid. With the whole sequence completed, the resin was dried in vacuo prior to cleavage. For cleavage and complete removal of acid-labile protection groups within the amino acids, the pre-cooled resin was suspended in a pre-cooled solution of trifluoroacetic acid (TFA)/triisopropylsilane (TIS)/H₂O (95/2.5/2.5 (v/v/v)) and agitated for 1 h (0.5 h in the case of ^{ε}K-^{ε}K-6-Ahx analog). The cleavage solution was drained and collected. The resin was washed once with TFA. The collected solutions were concentrated with nitrogen flow to approximately 1 mL and further mixed with 2 mL ethanol (EtOH). In the case of carriers with U-shape topology, he collected solutions were dried with nitrogen flow and the obtained pellets were dissolved in H₂O/EtOH (3:7 or 1:1 (v/v)). The clear solutions were either dialyzed against pure EtOH for 4 h, followed by dialysis against 10 mM HCl in H₂O for additional 24 h, or directly dialyzed against 10 mM HCl for 48 h. For dialysis, Spectra/Por® Dialysis Membranes (Carl Roth, Karlsruhe, Germany), molecular weight cut-off (MWCO) 1 or 2 kDa (depending on the MW of the synthesized sequence) were used. The samples were lyophilized. Characterization was performed by MALDI-TOF (matrix-assisted laser desorption ionization—time-of-flight)-MS (see Tab. 1-3) and ¹H-NMR spectroscopy (see below for some selected examples). Stock solutions were prepared in either pure EtOH, pure H₂O, or in an EtOH/H₂O mixture (in different ratios), depending on the solubility of the single LAF carriers. Synthesis of LAF containing carriers with Fmoc-protected artificial amino acid other than Stp are performed analogously.

B2 1621; K(K(8Oc)₂)₂-Stp, [C₁₂₆H₂₅₁N₁₅O₁₀]: ¹H-NMR (400 MHz, methanol-d4) δ (ppm)=0.90-0.97 (t, 24H, —CH₃, octanal), 1.30-1.77 (m, 154H, βγδ H lysine, βγδεz H Oc, —CH₂-octanal), 2.18-2.35 (m, 8H, —NH—CH₂—CH₂—NH— Stp), 2.53-2.69 (m, 4H, —CO—CH₂—CH₂—CO— Stp), 3.10-3.35 (m, 46H, —CH₂—N-LAF, α H Oc, ε H lysine, —CO—NH—CH₂—CH₂—NH— Stp), 4.19-4.31 (m, 3H, α H amino acids).

U1 1611; K(12Oc)-Stp-K(12Oc), [C₈₈H₁₇₇N₁₁O₇]: ¹H-NMR (400 MHz, methanol-d4) δ (ppm)=0.89-0.95 (t, 12H, —CH₃, dodecanal), 1.20-2.00 (m, 112H, βγδ H lysine, βγδεz H Oc, —CH₂-dodecanal), 2.19-2.27 (m, 8H, —NH—CH₂—CH₂—NH— Stp), 2.52-2.70 (m, 4H, —CO—CH₂—CH₂—CO— Stp), 3.1-3.70 (m, 28H, —CH₂—N-LAF, α H Oc, ε H lysine, —CO—NH—CH₂—CH₂—NH— Stp), 4.30-4.36 (2H, α H amino acid).

U3 1722; K(12Oc)₂-Stp2-K(12Oc)-K(12Oc), [C₁₇₀H₃₄₀N₂₀O₁₂]: ¹H-NMR (400 MHz, methanol-d4) δ (ppm)=0.89-0.95 (m, 24H, —CH₃, dodecanal), 1.15-1.87 (m, 218H, βγδ H lysine, βγδεz H Oc, —CH₂— dodecanal), 2.19-2.38 (m, 16H, —NH—CH₂—CH₂—NH— Stp), 2.61-2.66 (m, 8H, —CO—CH₂—CH₂—CO— Stp), 3.10-3.38 (m, 54H, —CH₂—N— LAF, α H Oc, ε H lysine, —CO—NH—CH₂—CH₂—NH— Stp), 4.24-4.37 (3H, α H amino acids).

U4 1716; K(12Oc)₂-Stp-K(K(12Oc)₂), [C₁₅₈H₃₁₅N₁₅O₁₀]: ¹H-NMR (400 MHz, methanol-d4) δ (ppm)=0.89-0.95 (m, 24H, —CH₃, dodecanal), 1.15-1.88 (m, 218H, βγδ H lysine, βγδεz H Oc, —CH₂-dodecanal), 2.16-2.39 (m, 8H, —NH—CH₂—CH₂—NH— Stp), 2.45-2.73 (m, 4H, —CO—CH₂—CH₂—CO— Stp), 3.10-3.36 (m, 46H, —CH₂—N— LAF, α H Oc, ε H lysine, —CO—NH—CH₂—CH₂—NH— Stp), 4.20-4.35 (3H, a H amino acids).

4.3 Kaiser Test as in-Process-Control of SPPS

Free amines of deprotected amino acids on the resin were determined qualitatively by the Kaiser test. A small sample of DCM washed resin was transferred into an Eppendorf reaction tube. One drop of each 80% (w/v) phenol in EtOH, 5% (w/v) ninhydrin in EtOH, and 20 μM potassium cyanide (KCN) in pyridine (mixture of 1 mL aqueous 1 mM KCN solution and 49 mL pyridine) were added. The tube was incubated under shaking at 99° C. for 4 min. The presence of free amines was indicated by blue color.

TABLE 1.1
LAF carriers with block/comb and T-shape topologies

| | | | | | | | | C |
|---|---|---|---|---|---|---|---|---|
| 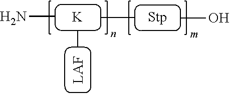 | | | | | | | | |
| | | | | | | | | T1 |
| 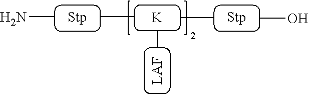 | | | | | | | | |
| | | | | | | | | T2 |
| 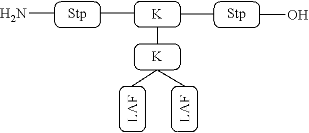 | | | | | | | | |
| | | | | sequence | chemical | protonatable | MW | mass |
| ID | topology | m | n | (N→C) | formula | amines | [Da] | found [Da] |
| Blocks (combs) | | | | | | | | |
| 1700 | C | 10 | — | Stp₁₀ | C_{12O}H₂₅₂N₅₀O₂₁ | 31 | 2731.67 | 2726.38 |
| 1701 | C | 10 | 2 | [K]₂-Stp₁₀ | C₁₃₂H₂₇₆N₅₄O₂₃ | 33 | 2988.02 | 2983.47 |
| 1704 | C | 10 | 2 | [K(12Oc)]₂- | C₁₉₆H₄₀₂N₅₆O₂₅ | 33 | 3943.74 | 3934.32 |
| | | | | Stp₁₀ | | | | |
| 1705 | C | 10 | 4 | [K(12Oc)]₄- | C₂₇₂H₅₅₂N₆₂O₂₉ | 35 | 5155.81 | n.d. |
| | | | | Stp₁₀ | | | | |
| 1706 | C | 10 | 6 | [K(12Oc)]₆- | C₃₄₈H₇₀₂N₆₈O₃₃ | 37 | 6367.89 | n.d. |
| | | | | Stp₁₀ | | | | |
| 1615 | C | 1 | 2 | [K(12Oc)]₂-Stp | C₈₈H₁₇₇N₁₁O₇ | 6 | 1501.42 | 1499.15 |
| 1616 | C | 1 | 4 | [K(12Oc)]₄-Stp | C₁₆₄H₃₂₇N₁₇O₁₁ | 8 | 2713.46 | 2700.84 |
| 1617 | C | 1 | 6 | [K(12Oc)]₆-Stp | C₂₄₀H₄₇₇N₂₃O₁₅ | 10 | 3925.50 | 3912.56 |
| 1707 | C | 2 | 4 | [K(12Oc)]₄-Stp₂ | C₁₇₆H₃₅₂N₂₂O₁₃ | 11 | 2984.89 | 2981.51 |
| 1708 | C | 2 | 2 | [K(12Oc)]₂-Stp₂ | C₁₀₀H₂₀₂N₁₆O₉ | 9 | 1772.82 | 1765.02 |
| 1709 | C | 4 | 4 | [K(12Oc)]₄-Stp₄ | C₂₀₀H₄₀₂N₃₂O₁₇ | 17 | 3527.62 | 3516.46 |
| Lipidic center / T-shapes | | | | | | | | |
| 1714 | T1 | | | Stp-[K(12Oc)]₂- | C₁₀₀H₂₀₂N₁₆O₉ | 9 | 1772.82 | 1765.02 |
| | | | | Stp | | | | |
| 1715 | T2 | | | Stp- | C₁₀₀H₂₀₂N₁₆O₉ | 9 | 1772.82 | 1765.30 |
| | | | | K[K(12Oc)₂]- | | | | |
| | | | | Stp | | | | |

K, lysine;
MW, molecular weight;
n.d., not detectable;
Stp, succinyl-tetraethylenepentamine;
12Oc, lipo amino fatty acid (LAF) based on 8-aminooctanoic acid and 2 dodecyl chains.

TABLE 1.2
LAF carriers with bundle topology

| 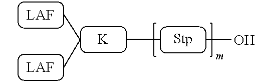 | | | | | | | B1 |
|---|---|---|---|---|---|---|---|
| | | | | | | | B2 |
| 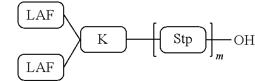 | | | | | | | |
| | | | sequence | chemical | protonatable | MW | mass found |
| ID | topology | m | (N→C) | formula | amines | [Da] | [Da] |
| Bundles | | | | | | | |
| 1614 | B1 | 1 | K(12Oc)₂-Stp | C₈₂H₁₆₅N₉O₆ | 5 | 1373.24 | 1370.00 |
| 1729 | B1 | 1 | K(8Oc)₂-Stp | C₆₆H₁₃₃N₉O₆ | 5 | 1148.85 | 1146.11 |
| 1710 | B1 | 2 | K(12Oc)₂-Stp₂ | C₉₄H₁₉₀N₁₄O₈ | 8 | 1644.64 | 1641.48 |
| 1712 | B1 | 1 | K(DodOc)₂-Stp | C₅₈H₁₁₃N₉O₈ | 3 | 1064.6 | 1062.26 |
| 1613 | B2 | 1 | K[K[12Oc)₂]₂- | C₁₅₈H₃₁₅N₁₅O₁₀ | 7 | 2585.29 | 2580.17 |
| | | | Stp | | | | |
| 1621 | B2 | 1 | K[K[8Oc)₂]₂-Stp | C₁₂₆H₂₅₁N₁₅O₁₀ | 7 | 2136.45 | 2133.46 |
| 1752 | B2 | 1 | K[K(12Bu)₂]₂- | C₁₄₂H₂₈₃N₁₅O₁₀ | 7 | 2360.92 | 2355.67 |
| | | | Stp | | | | |
| 1753 | B2 | 1 | K[K(16Bu)₂]₂- | C₁₇₄H₃₄₇N₁₅O₁₀ | 7 | 2809.79 | 2803.18 |
| | | | Stp | | | | |
| 1754 | B2 | 1 | K[K(12He)₂]₂- | C₁₅₁H₃₀₀N₁₄O₁₀ | 7 | 2472.15 | 2468.57 |
| | | | Stp | | | | |
| 1755 | B2 | 1 | K[K(14He)₂]₂- | C₁₆₇H₃₃₂N₁₄O₁₀ | 7 | 2696.58 | 2691.38 |
| | | | Stp | | | | |
| 1762 | B2 | 1 | K[K(10Oc)₂]₂- | C₁₄₁H₂₈₁N₁₅O₁₀ | 7 | 2346.89 | 2339.78 |
| | | | Stp | | | | |
| 1713 | B2 | 2 | K[K[12Oc)₂]₂- | C₁₇₀H₃₄₀N₂₀O₁₂ | 10 | 2856.72 | 2849.98 |
| | | | Stp₂ | | | | |
| 1730 | B2 | 2 | K[K[8Oc)₂]₂-Stp₂ | C₁₃₈H₂₇₆N₂₀O₁₂ | 10 | 2407.85 | 2402.25 |

DodOc, 8-dodecanamido-octanoic acid;
K, lysine;
MW, molecular weight;
Stp, succinyl-tetraethylenepentamine;
12Bu, lipo amino fatty acid (LAF) based on 4-aminobutanoic acid and 2 dodecyl chains;
16 Bu, LAF based on 4-aminobutanoic acid and 2 hexadecyl chains;
12He, LAF based on 6-aminohexanoic acid and 2 dodecyl chains;
14He, LAF based on 6-aminohexanoic acid and 2 tetradecyl chains;
8Oc, LAF based on 8-aminooctanoic acid and 2 octyl chains;
10Oc, LAF based on 8-aminooctanoic acid and 2 decyl chains;
12Oc, LAF based on 8-aminooctanoic acid and 2 dodecyl chains.

TABLE 1.3
LAF carriers with U-shape topology

| | |
|---|---|
| | U1 |
| 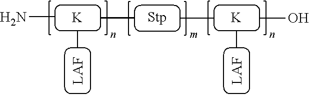 | |
| | U2 |
| 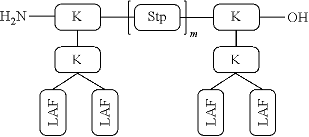 | |
| | U3 |
| 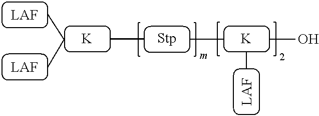 | |
| | U4 |
| 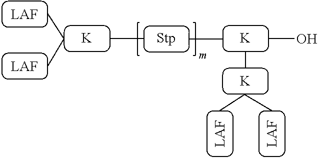 | |

| | | | | | | | | mass |
|---|---|---|---|---|---|---|---|---|
| | | | | sequence | chemical | protonatable | MW | found |
| ID | topology | m | n | (N→C) | formula | amines | [Da] | [Da] |
| Lipidic ends / U-shapes | | | | | | | | |
| 1611 | U1 | 1 | 1 | K(12Oc)-Stp- | C₈₈H₁₇₇N₁₁O₇ | 6 | 1501.42 | 1499.15 |
| | | | | K(12Oc) | | | | |
| 1763 | U1 | 1 | 1 | K(12Bu)-Stp- | C₈₀H₁₆₁N₁₁O₇ | 6 | 1389.24 | 1386.06 |
| | | | | K(12Bu) | | | | |
| 1764 | U1 | 1 | 1 | K(16Bu)-Stp- | C₉₆H₁₉₃N₁₁O₇ | 6 | 1613.67 | 1609.81 |
| | | | | K(16Bu) | | | | |
| 1765 | U1 | 1 | 1 | K(12He)-Stp- | C₈₄H₁₆₉N₁₁O₇ | 6 | 1445.35 | 1442.04 |
| | | | | K(12He) | | | | |
| 1766 | U1 | 1 | 1 | K(14He)-Stp- | C₉₂H₁₈₅N₁₁O₇ | 6 | 1557.56 | 1554.54 |
| | | | | K(14He) | | | | |
| 1746 | U1 | 1 | 1 | K(8Oc)-Stp- | C₇₂H₁₄₅N₁₁O₇ | 6 | 1277.02 | 1274.22 |
| | | | | K(8Oc) | | | | |
| 1747 | U1 | ^{ε}K* | 1 | K(12Oc)-(^{ε}K-^{ε}K- | C₉₄H₁₈₇N₁₁O₈ | 5 | 1599.6 | 1595.85 |
| | | | | 6-Ahx)-K(12Oc) | | | | |
| 1748 | U1 | 1 | 1 | K(DodOc)-Stp- | C₆₄H₁₂₅N₁₁O₉ | 4 | 1192.77 | 1189.97 |
| | | | | K(DodOc) | | | | |
| 1718 | U1 | 1 | 2 | [K(12Oc)]₂-Stp- | C₁₆₄H₃₂₇N₁₇O₁₁ | 8 | 2713.53 | 2707.03 |
| | | | | [K(12Oc)]₂ | | | | |
| 1681 | U1 | 2 | 1 | K(12Oc)-Stp₂- | C₁₀₀H₂₀₂N₁₆O₉ | 9 | 1772.82 | 1769.04 |
| | | | | K(12Oc) | | | | |
| 1719 | U1 | 2 | 2 | [K(12Oc)]₂- | C₁₇₆H₃₅₂N₂₂O₁₃ | 11 | 2984.89 | 2977.11 |
| | | | | Stp₂-[K(12Oc)]₂ | | | | |
| 1758 | U1 | 2 | 2 | [K(12Bu)]₂- | C₁₆₀H₃₂₀N₂₂O₁₃ | 11 | 2760.46 | 2753.81 |
| | | | | Stp₂-[K(12Bu)]₂ | | | | |
| 1759 | U1 | 2 | 2 | [K(16Bu)]₂- | C₁₉₂H₃₈₄N₂₂O₁₃ | 11 | 3209.33 | 3202.05 |
| | | | | Stp₂-[K(16Bu)]₂ | | | | |
| 1760 | U1 | 2 | 2 | [K(12He)]₂- | C₁₆₈H₃₃₆N₂₂O₁₃ | 11 | 2872.68 | 2865.70 |
| | | | | Stp₂-[K(12He)]₂ | | | | |
| 1761 | U1 | 2 | 2 | [K(14He)]₂- | C₁₈₄H₃₆₈N₂₂O₁₃ | 11 | 3097.11 | 3089.54 |
| | | | | Stp₂-[K(14He)]₂ | | | | |
| 1745 | U1 | 2 | 2 | [K(8Oc)]₂-Stp₂- | C₁₄₄H₂₈₈N₂₂O₁₃ | 11 | 2536.03 | 2530.05 |
| | | | | [K(8Oc)]₂ | | | | |
| 1720 | U2 | 1 | | K[K(12Oc)₂]- | C₁₆₄H₃₂₇N₁₇O₁₁ | 8 | 2713.53 | 2707.38 |
| | | | | Stp- | | | | |
| | | | | K[K(12Oc)₂] | | | | |
| 1721 | U2 | 2 | | K[K(12Oc)₂]- | C₁₇₆H₃₅₂N₂₂O₁₃ | 11 | 2984.89 | 2977.86 |
| | | | | Stp₂- | | | | |
| | | | | K[K(12Oc)₂] | | | | |
| 1612 | U3 | 1 | | K(12Oc)₂-Stp- | C₁₅₈H₃₁₅N₁₅O₁₀ | 7 | 2585.28 | 2580.61 |
| | | | | K(12Oc)- | | | | |
| | | | | K(12Oc) | | | | |
| 1620 | U3 | 1 | | K(8Oc)₂-Stp- | C₁₂₆H₂₅₁N₁₅O₁₀ | 7 | 2136.44 | 2131.87 |
| | | | | K(8Oc)-K(8Oc) | | | | |
| 1722 | U3 | 2 | | K(12Oc)₂-Stp₂- | C₁₇₀H₃₄₀N₂₀O₁₂ | 10 | 2856.72 | 2849.99 |
| | | | | K(12Oc)- | | | | |
| | | | | K(12Oc) | | | | |
| 1731 | U3 | 2 | | K(8Oc)₂-Stp₂- | C₁₃₈H₂₇₆N₂₀O₁₂ | 10 | 2407.85 | 2403.20 |
| | | | | K(8Oc)-K(8Oc) | | | | |
| 1724 | U3 | 1 | | K(DodOc)₂-Stp- | C₁₁₀H₂₁₁N₁₅O₁₄ | 3 | 1967.99 | 1963.33 |
| | | | | K(DodOc)- | | | | |
| | | | | K(DodOc) | | | | |
| 1725 | U3 | 2 | | K(DodOc)₂- | C₁₂₂H₂₃₆N₂₀O₁₆ | 6 | 2239.35 | 2234.52 |
| | | | | Stp₂-K(DodOc)- | | | | |
| | | | | K(DodOc) | | | | |
| 1716 | U4 | 1 | | K(12Oc)₂-Stp- | C₁₅₈H₃₁₅N₁₅O₁₀ | 7 | 2585.35 | 2579.90 |
| | | | | K[K(12Oc)₂] | | | | |
| 1749 | U4 | 1 | | K(DodOc)₂-Stp- | C₁₁₀H₂₁₁N₁₅O₁₄ | 3 | 1967.99 | 1963.53 |
| | | | | K[K(DodOc)₂] | | | | |
| 1717 | U4 | 2 | | K(12Oc)₂-Stp₂- | C₁₇₀H₃₄₀N₂₀O₁₂ | 10 | 2856.72 | 2849.90 |
| | | | | K[K(12Oc)₂] | | | | |
| 1732 | U4 | 2 | | K(8Oc)₂-Stp₂- | C₁₃₈H₂₇₆N₂₀O₁₂ | 10 | 2407.85 | 2402.92 |
| | | | | K[K(8Oc)₂] | | | | |
| 1751 | U4 | 2 | | K(DodOc)₂- | C₁₂₈H₂₄₈N₂₂O₁₇ | 6 | 2367.53 | 2361.96 |
| | | | | Stp₂- | | | | |
| | | | | K[K(DodOc)₂] | | | | |

6-Ahx, 6-aminohexanoic acid;
DodOc, 8-dodecanamido-octanoic acid;
K, lysine;
EK, lysine with free a-amino group and peptide bond at &-amino position;
MW, molecular weight;
Stp, succinyl-tetraethylenepentamine;
12Bu, lipo amino fatty acid (LAF) based on 4-aminobutanoic acid and 2 dodecyl chains;
16Bu, LAF based on 4-aminobutanoic acid and 2 hexadecyl chains;
12He, LAF based on 6-aminohexanoic acid and 2 dodecyl chains;
14He, LAF based on 6-aminohexanoic acid and 2 tetradecyl chains;
8Oc, LAF based on 8-aminooctanoic acid and 2 octyl chains;
12Oc, LAF based on 8-aminooctanoic acid and 2 dodecyl chains.
*(^{ε}K-^{ε}K-6-Ahx) instead of Stp

### 4.4. Synthesis of Carriers Containing Different Polar Building Blocks (Stp Analogs).

The carriers containing different artificial amino acid building blocks (see Table 2) were synthesized in analogous fashion to existing LAF carriers 1611 (12Oc-U1-1:2), 1719 (12Oc-U1-2:4) and 1752 (12Bu-B2-1:4) via standard Fmoc solid-phase assisted peptide synthesis as described in sections 4.1 and 4.2. Other protected artificial amino acids were used instead of Fmoc-Stp(Boc₃)-OH without changes to the coupling method. However, for the modification with LAFs, the respective LAF and DIPEA were dissolved in DCM supplemented with 2% (v/v) Triton X-100, PyBOP and HOBt were dissolved in DMF. The resin was incubated with these solutions under constant agitation for at least 72 h and the reaction time was prolonged if the reaction was incomplete (monitoring via Kaiser test, section 4.3. Stock solutions of synthesized carriers (hydrochloride salts) were prepared in either EtOH, H₂O, or in an EtOH/H₂O mixture (in different ratios), optionally pH-adjusted with dilute aqueous HCl, depending on the solubility of the single LAF carriers.

TABLE 2
Synthesis of carriers containing different polar building blocks (Stp analogs)
replacing Stp.

| | |
|---|---|
| | U1 |
| 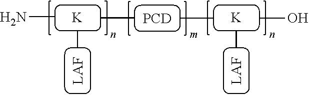 | |
| | B2 |
| 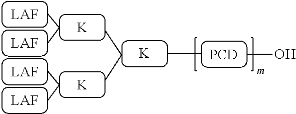 | |

| | | | | | | | | mass |
|---|---|---|---|---|---|---|---|---|
| | | | | | chemical | protonatable | MW | found |
| ID | topology | m | n | sequence (N->C) | formula | amines | [Da] | [Da] |
| Lipidic ends / U-shapes | | | | | | | | |
| 1840 | U1 | 1 | 1 | K(12Oc)-dmGtp- | C₉₁H₁₈₃N₁₁O₇ | 6 | 1543.54 | 1540.35 |
| | | | | K(12Oc) | | | | |
| 1841 | U1 | 1 | 1 | K(12Oc)-chGtp-K(12Oc) | C₉₄H₁₈₇N₁₁O₇ | 6 | 1583.60 | 1580.63 |
| 1842 | U1 | 1 | 1 | K(12Oc)-dGtp-K(12Oc) | C₈₈H₁₇₇N₁₁O₈ | 6 | 1517.45 | 1513.63 |
| 1843 | U1 | 1 | 1 | K(12Oc)-Htp-K(12Oc) | C₉₂H₁₈₃N₁₁O₇ | 6 | 1555.55 | 1551.69 |
| 1844 | U1 | 1 | 1 | K(12Oc)-Stt-K(12Oc) | C₈₆H₁₇₂N₁₀O₇ | 5 | 1458.39 | 1454.76 |
| 1845 | U1 | 1 | 1 | K(12Oc)-GEIPA- | C₈₉H₁₇₈N₁₀O₇ | 5 | 1500.47 | 1496.72 |
| | | | | K(12Oc) | | | | |
| 1858 | U1 | 2 | 2 | [K(12Oc)]₂-dmGtp₂- | C₁₈₂H₃₆₄N₂₂O₁₃ | 11 | 3069.06 | 3061.66 |
| | | | | [K(12Oc)]₂ | | | | |
| 1859 | U1 | 2 | 2 | [K(12Oc)]₂-chGtp₂- | C₁₈₈H₃₇₂N₂₂O₁₃ | 11 | 3149.19 | 3141.91 |
| | | | | [K(12Oc)]₂ | | | | |
| 1861 | U1 | 2 | 2 | [K(12Oc)]₂-Htp₂- | C₁₈₄H₃₆₄N₂₂O₁₃ | 11 | 3093.08 | 3085.91 |
| | | | | [K(12Oc)]₂ | | | | |
| 1862 | U1 | 2 | 2 | [K(12Oc)]₂-Stt₂- | C₁₇₂H₃₄₂N₂₀O₁₃ | 9 | 2898.76 | 2892.03 |
| | | | | [K(12Oc)]₂ | | | | |
| Bundles | | | | | | | | |
| 1864 | B2 | 1 | — | K[K(12Bu)₂]₂-dmGtp | C₁₄₅H₂₈₉N₁₅O₁₀ | 7 | 2403.00 | 2397.30 |
| 1865 | B2 | 1 | — | K[K(12Bu)₂]₂-chGtp | C₁₄₈H₂₉₃N₁₅O₁₀ | 7 | 2443.07 | 2436.05 |
| 1867 | B2 | 1 | — | K[K(12Bu)₂]₂-Htp | C₁₄₆H₂₈₉N₁₅O₁₀ | 7 | 2415.01 | 2409.36 |

chGtp, 1,1-cyclohexanediacetyl-tetraethylenepentamine;
dGtp, diglycolyl-tetraethylenepentamine;
dmGtp, dimethyl-glutaryl-tetraethylenepentamine;
GEIPA, glutaryl-3,3'-ethylenediiminodipropylamine;
Htp, cyclohexane-1,2-dicarboxyl-tetraethylenepentamine;
K, lysine;
MW, molecular weight;
Stt, succinyl-triethylenetetramine;
12Bu, lipo amino fatty acid (LAF) based on 4-aminobutanoic acid and 2 dodecyl chains;
12Oc, LAF based on 8-aminooctanoic acid and 2 dodecyl chains.

### 4.5 Synthesis of Novel Carriers Connecting Stp and LAF Via Ornithine as BC.

The novel carriers (Table 3.1) were synthesized via standard Fmoc solid-phase assisted peptide synthesis as described in sections 4.1 and 4.2. In comparison to existing carriers, Stp and LAF were connected via ornithine instead of lysine as branching connector. For introduction of symmetrical branching points Fmoc-L-Orn(Fmoc)-OH was used, whereas asymmetric branching points were introduced by using Fmoc-L-Orn(Dde)-OH. Stock solutions of synthesized carriers (hydrochloride salts) were prepared in either EtOH, H₂O, or in an EtOH/H₂O mixture (in different ratios), optionally pH adjusted with dilute aqueous HCl, depending on the solubility of the single LAF carriers.

TABLE 3.1
LAF carriers of different topologies containing ornithine as branching connector.

| | | | | | chemical | | |
|---|---|---|---|---|---|---|---|
| ID | code | m | n | sequence (N−>C) | formula | MW calc. | M [found] |
| 1813 | 8Oc-B2-1:4 | 1 | — | [Orn(8Oc)₂]₂-Stp | C₁₂₃H₂₄₅N₁₅O₁₀ | 2094.41 | 2092.8 |
| 1814 | 12Bu-B2-1:4 | 1 | — | [Orn(12Bu)₂]₂-Stp | C₁₃₉H₂₇₇N₁₅O₁₀ | 2318.84 | 2312.14 |
| 1827 | 12Oc-U1-1:2 | 1 | 1 | Orn(12Oc)-Stp-Orn(12Oc) | C₈₆H₁₇₃N₁₁O₇ | 1473.40 | 1483.74 |

Stp, succinyl-tetraethylenepentamine; Orn, ornithine; 8Oc, LAF based on 8-amino octanoic acid and two octyl chains; 12Bu, LAF based on 4-amino butanoic acid and two dodecyl chains; 12Oc, LAF based on 8-amino octanoic acid and two dodecyl chains; MW, molecular weight.

### 4.6 Synthesis of Bioreducible Carriers Containing Disulfide Building Blocks.

The bioreducible disulfide containing LAF carriers (Table 3.2) were synthesized in analogous fashion to existing non-reducible LAF carriers 1621 (8Oc-B2-1:4), 1730 (8Oc-B2-2:4) and 1611 (12Oc-U1-1:2) via standard Fmoc solid-phase assisted peptide synthesis as described in sections 4.1 and 4.2. For introduction of bioreducible cleavage sites, the Fmoc-SPPS compatible cystamine disulfide building block [Klein et al., Nanoscale 2016 8, 18098-18104] was precisely positioned at indicated locations in the sequence. For coupling, 4 eq. of Fmoc-cystamine disulfide building block and 4 eq. of PyBOP were dissolved in DMF, followed by addition of 8 eq. of DIPEA and subsequent transfer to the reactor.

TABLE 3.2
LAF carriers of different topologies containing disulfide building blocks for bioreducibility.

| | | | | | | | | |
|---|---|---|---|---|---|---|---|---|
| | | | | | | | | Mass |
| | | | | Sequence | | Protonable | | found |
| ID | Topology | m | n | (N→C) | Chemical formula | amines | MW [Da] | [Da] |

Bundles

| | | | | | | | | |
|---|---|---|---|---|---|---|---|---|
| 1792 | B2 | 1 | — | [(8Oc)₂)-K- | C₁₄₂H₂₇₉N₁₉O₁₄S₄ | 7 | 2605.15 | 2598.83 |
| | | | | ssbb]₂-K-Stp | | | | |
| 1824 | B2 | 2 | — | [(8Oc)₂)-K- | C₁₅₄H₃₀₄N₂₄O₁₆S₄ | 10 | 2876.52 | 2869.16 |
| | | | | ssbb]₂-K- | | | | |
| | | | | Stp₂ | | | | |

Lipidic ends/U-shapes

| | | | | | | | | |
|---|---|---|---|---|---|---|---|---|
| 1821 | U1 | 1 | 1 | K(ssbb- | C₁₀₄H₂₀₅N₁₅O₁₁S₄ | 6 | 1970.12 | 1965.63 |
| | | | | 12Oc)-Stp- | | | | |
| | | | | K(ssbb- | | | | |
| | | | | 12Oc) | | | | |

ID, identification number; MW, molecular weight; Stp, succinyl-tetraethylene-pentamine; K, lysine; ssbb, Fmoc-SPPS compatible disulfide building block; 8Oc, LAF based on 8-amino octanoic acid and two octyl chains; 12Oc, LAF based on 8-amino octanoic acid and two dodecyl chains.
Analogous pairs (disulfide analog - original) are 1792 - 1621, 1824 - 1730 and 1821 - 1611.

### 4.7 Synthesis of Novel 2-Arm Carrier 1851 Containing εK as Polar Cationizable Domain (PCD) Instead of Stp.

The novel 2-arm carrier (Table 3.3) was synthesized via standard Fmoc solid-phase assisted peptide synthesis as described in sections 4.1 and 4.2. In order to create two symmetric arms, a Fmoc-L-Lysine(Fmoc)-OH was used as C-terminal starting amino acid and double equivalents of following amino acids and coupling reagents were used to synthesize the 2-arm backbone. Boc-L-Lysine(Fmoc)-OH was used to enable peptide backbone coupling via the ε-amine of the lysine side chain instead of its α-amine.

TABLE 3.3
2-arm carrier 1851 containing εK as polar cationizable domain (PCD) instead of Stp.

| | | STP | | | | |
|---|---|---|---|---|---|---|
| | | equivalent/ | | Chemical | | |
| ID | topology | LAF ratio | Sequence (N −> C) | formula | MW cal. | MW found |
| 1851 | sym. 2-arm (~U1/U4) | 2:4 | K[K₂-K(12Oc)₂]₂ | C₁₇₀H₃₃₈N₁₈O₁₂ | 2824.64 | 2819.915 |

K₂, two lysines connected by ε-amino peptide bond; sym. 2-arm (~U1/U4), a symmetrical two-arm structure resembling the related topology in U1 or U4 structures.

### 5. Analytical Methods

5.1 Proton (¹H)- and Carbon (¹³C)-NMR Spectroscopy

¹H- and ¹³C-NMR spectra were recorded using an AVANCE III HD 400 (400 MHz) by Bruker with a 5 mm CPPBBO probe. Instead of using TMS as an internal standard, all signals were calibrated to the residual signal of chloroform-d (CDCl₃) or methanol-d4, which served as the solvent. Chemical shifts are reported in ppm and refer to the solvent as internal standard. Integration was performed manually. The spectra were analyzed using MestreNova (version 9.0 by MestReLab Research).

### 5.2 ESI and EI Mass Spectrometry

The samples were dissolved in chloroform to a concentration of 1 mg/mL. ESI-MS and EI-MS were carried out using a Thermoscientific LTQ FT Ultra Fourier transform ion cyclotron and an IonMax source. Data is either shown as [M+X] after positive ionization (ESI-MS) or fragmented radicals of the desired product (EI-MS).

### 5.3 MALDI-TOF Mass Spectrometry

The matrix solution for MALDI-TOF mass spectrometry contains 10 mg/mL Super-DHB (90/10 (m/m) mixture of 2,5-dihydroxybenzoic acid and 2-hydroxy-5-methoxybenzoic acid) in 69.93/30/0.07 (v/v/v) H₂O/acetonitrile/trifluoroacetic acid. 1 μL of matrix solution was spotted on an MTP AnchorChip (Bruker Daltonics, Germany). After crystallization 1 μL of sample solution (1 mg/mL in water) was added onto the matrix spot. Samples were analyzed using an Autoflex II mass spectrometer (Bruker Daltonics, Germany). All spectra were recorded in positive ion mode.

6. Physicochemical and Biological Characterization of Nucleic Acid Nanoparticles Formed with LAF Containing Carriers
6.1 pDNA, mRNA, and siRNA Polyplex Formation

The nucleic acid and calculated amounts of aminolipid at indicated N/P (nitrogen/phosphate) ratios were diluted in separate tubes of HBG (20 mM of HEPES, 5% (w/v) glucose, pH 7.4). All secondary amines of the Stp (succinyl-tetraethylenepentamine) units, terminal amines and the tertiary amines of the LAFs were considered in the N/P ratio calculations. Equal volumes of nucleic acid solution and aminolipid solution were mixed by rapid pipetting and incubated 40 min at RT in a closed Eppendorf reaction tube. The final concentration of nucleic acid in the polyplex solution was 12.5 μg/mL for mRNA (CleanCap® FLuc mRNA (5moU); Trilink Biotechnologies, San Diego, CA, USA), 10 μg/mL for pDNA (pCMVLuc; Plasmid Factory GmbH, Bielefeld, Germany), and 25 μg/mL for siRNA.

siRNA duplexes (Axolabs GmbH, Kulmbach, Germany): eGFP-targeting siRNA (siGFP) (sense strand: 5′-AuAucAuGGccGAcAAGcAdTsdT-3′; SEQ ID NO: 1); antisense strand: 5′-UGCUUGUCGGCcAUGAuAUdTsdT-3′; SEQ ID NO: 2) for silencing of eGFPLuc; control siRNA (siCtrl) (sense strand: 5′-AuGuAuuGGccuGuAuuAGdTsdT-3′; SEQ ID NO: 3; antisense strand: 5′-CuAAuAcAGGCcAAuAcAUdTsdT-3′; SEQ ID NO: 4); small letters indicate 2′methoxy modifications; “s” indicates phosphorothioate linkages; “dT” refers to deoxythymidine.

6.2 Polyplex Formation for mRNA/pDNA Co-Delivery

CleanCap® mCherry mRNA (5moU) (Trilink Biotechnologies, San Diego, CA, USA) and pEGFP-N1 pDNA (Clontech Laboratories, Inc. (now Takara Bio USA, Inc., San Jose, CA, USA)) at a fixed weight ratio of 1:1 were mixed and diluted in HBG. Calculated amounts of aminolipid at indicated N/P (nitrogen/phosphate) ratios (see above) were diluted in separate tubes of HBG. Equal volumes of nucleic acid solution and aminolipid solution were mixed by rapid pipetting and incubated 40 min at RT in a closed Eppendorf reaction tube. The final concentration of nucleic acid in the polyplex solution was 5 μg/mL (2.5 μg/mL mCherry mRNA plus 2.5 μg/mL pEGFP-N1 pDNA).

6.3 mRNA and siRNA Lipid Nanoparticle (LNP) Formulations

LNPs were formulated by mixing an acidic aqueous phase containing mRNA or siRNA with an EtOH phase containing ionizable and helper lipids (v/v 3:1) by rapid pipetting. The aqueous phase was prepared in citrate buffer (10 mM, pH 4.0) with mRNA (CleanCap® FLuc mRNA (5moU); Trilink Biotechnologies, San Diego, CA, USA) or siRNA (Axolabs GmbH, Kulmbach, Germany). The EtOH phase includes a mixture of cholesterol (Sigma-Aldrich, Munich, Germany), 1,2-Distearoyl-sn-glycero-3-phosphocholine (DSPC; Avanti Polar Lipids, Alabaster, AL, USA), 1,2-dimyristoyl-rac-glycero-3-methoxypolyethylene glycol-2000 (DMG-PEG 2000; Avanti Polar Lipids, Alabaster, AL, USA) and an ionizable lipid at predetermined molar and N/P ratios shown in Table A. For post-functionalization of LNPs, 1,2-distearoyl-sn-glycero-3-phosphoethanolamine-N—[azido(polyethylene glycol)-2000](DSPE-PEG(2000) Azide; Avanti Polar Lipids, Alabaster, AL, USA) was incorporated additionally, see Table B. The ionizable lipid Dlin-MC3-DMA was obtained from MedChemExpress and SM-102 from Biosynth Carbosynth. Other ionizable lipids (LAF carriers) were synthesized according to the previously described method. The aqueous and EtOH solutions were rapidly mixed by pipetting for 30 sec and then incubated at RT for 10 min to allow LNP assembly. The formulated LNPs were dialyzed against HBG buffer in 1 kDa MWCO tubes at 4° C. for 2 h. For post-functionalization of LNPs with human transferrin (hTf, 1.5 eq. of hTf-PEG12-DBCO per DSPE-PEG(2000)-Azide were added to azide containing LNPs and the solution was incubated at RT for 4 h.

TABLE A
composition of mRNA LNPs or siRNA LNPs

| | | | w/w | w/w |
|---|---|---|---|---|
| | Molar ratio [mol %] | | ratio total | ratio total |
| Ionizable | Chol/DSPC/PEG DMG/ | N/P | lipids/ | lipids/ |
| lipid | Ionizable lipid | ratio | mRNA | siRNA |
| DLin-MC3 | 38.5/10/1.5/50 | 4.5 | 15 | 16 |
| SM-102 | 38.5/10/1.5/50 | 4.5 | 16 | 17 |
| 1612 | 59.9/15.6/2.3/22.2 | 9 | 17 | — |
| 1613 | 59.9/15.6/2.3/22.2 | 9 | 17 | — |
| 1620 | 59.9/15.6/2.3/22.2 | 9 | 16 | — |
| 1621 | 59.9/15.6/2.3/22.2 | 9 | 16 | 17 |
| 1716 | 59.9/15.6/2.3/22.2 | 9 | 17 | — |
| 1718 | 61.6/16/2.4/20 | 9 | 17 | — |
| 1720 | 61.6/16/2.4/20 | 9 | 17 | — |
| 1752 | 59.9/15.6/2.3/22.2 | 9 | 17 | 18 |
| 1753 | 59.9/15.6/2.3/22.2 | 9 | 18 | 20 |
| 1754 | 59.9/15.6/2.3/22.2 | 9 | 17 | 18 |
| 1755 | 59.9/15.6/2.3/22.2 | 9 | 18 | 19 |
| 1762 | 59.9/15.6/2.3/22.2 | 9 | — | 18 |

TABLE B

| | Molar ratio [mol %] | | w/w |
|---|---|---|---|
| Ionizable | Chol/DSPC/PEG DMG/DSPE-PEG- | N/P | ratio total |
| lipid | N₃/Ionizable lipid | ratio | lipids/mRNA |
| 1612 | 59.7/15.6/1/1.5/22.2 + 1.5 eq. hTf | 9 | 17 |
| 1621 | 59.7/15.6/1/1.5/22.2 + 1.5 eq. hTf | 9 | 16 |
| 1716 | 59.7/15.6/1/1.5/22.2 + 1.5 eq. hTf | 9 | 16 |

6.4 Cas9 mRNA/sgRNA Polyplex Formation and RNP Polyplex Formation

For Cas9 mRNA/sgRNA polyplexes, CleanCap® Cas9 mRNA (5moU) (Trilink Biotechnologies, San Diego, CA, USA) and sgRNA at a fixed weight ratio of 1:1 were mixed and diluted in HBG.

For RNP polyplexes, Cas9 protein and sgRNA were mixed at molar ratio 1:1 and diluted in HBG after 15 min incubation.

Calculated amounts of aminolipid at indicated N/P (nitrogen/phosphate) ratios (see above) were diluted in separate tubes of HBG. Equal volumes of nucleic acid solution and aminolipid solution were mixed by rapid pipetting and incubated 40 min at RT in a closed Eppendorf reaction tube.

For RNP polyplexes, a final concentration of 375 nM RNP (62 μg/mL Cas9 protein plus 12 μg/mL sgRNA) was obtained. For Cas9 mRNA/sgRNA polyplexes, final concentrations of nucleic acid in the different experiments were 5 μg/mL, 12 μg/mL or 12.5 μg/mL total RNA (mRNA/sgRNA weight ratio 1:1), respectively.

(a) Single guide RNAs sgGFP1 and sgDMDex23 (both sgRNAs, 2′ O-methyl and phosphorothioate modification on the first three and penultimate three RNA bases) were purchased from Integrated DNA Technologies (IDT; Coralville, IA, USA). The sequences of sgGFP1 and sgDMDex23 are shown below.

sgGFP1 sequence (IDT; SEQ ID NO: 5):
5′ mG*mA*mC*rCrArGrGrArUrGrGrGrCrArCrCrArCrCrCrGr
UrUrUrUrArGrArGrCrUrArGrArArArUrArGrCrArArGrUrUr
ArArArArUrArArGrGrCrUrArGrUrCrCrGrUrUrArUrCrArAr
CrUrUrGrArArArArArGrUrGrGrCrArCrCrGrArGrUrCrGrGr
UrGrCmU*mU*mU*rU 3′
sgDMDex23 sequence (IDT; SEQ ID NO: 6):
5′ mA*mU*mU*rUrCrArGrGrUrArArGrCrCrGrArGrGrUrUrGr
UrUrUrUrArGrArGrCrUrArGrArArArUrArGrCrArArGrUrUr
ArArArArUrArArGrGrCrUrArGrUrCrCrGrUrUrArUrCrArAr
CrUrUrGrArArArArArGrUrGrGrCrArCrCrGrArGrUrCrGrGr
UrGrCmU*mU*mU*rU 3′
‘r_’ = RNA bases (e.g. ‘rA’)
‘m_*’ = phosphorothioated 2′-O-methyl RNA bases e.g. ‘mA*

6.5 Cas9 mRNA/sgGFP2/ssDNA Polyplex Formation for eGFP to BFP Conversion Study

CleanCap® Cas9 mRNA (5moU) (Trilink Biotechnologies, San Diego, CA, USA) and sgGFP (IDT, Coralville, IA, USA) at a fixed weight ratio of 1:1 were mixed with indicated weight ratios of total RNA (Cas9 mRNA and sgGFP2) to the single stranded DNA template (IDT, Coralville, IA, USA) and diluted in HBG. Calculated amounts of LAF carrier 1611 resulting in an N/P ratio of 18 were diluted in separate tubes of HBG. Equal volumes of nucleic acid solution and 1611 solution were mixed by rapid pipetting and incubated 40 min at RT in a closed Eppendorf reaction tube. The final total concentration of nucleic acid in the polyplex solution was 7.5 μg/mL.

Single guide sgGFP2 (sgRNA, 2′ O-methyl and phosphorothioate modification on the first three and penultimate three RNA bases) and single stranded DNA (ssDNA; first two and penultimate two DNA bases are phosphothioated) were purchased from IDT (Coralville, IA, USA), the sequences of sgGFP2 and ssDNA are shown below.

sgGFP2 sequence (IDT; SEQ ID NO: 7):
5′ mG*mC*mU*rGrArArGrCrArCrUrGrCrArCrGrCrCrGrUrGr
UrUrUrUrArGrArGrCrUrArGrArArArUrArGrCrArArGrUrUr
ArArArArUrArArGrGrCrUrArGrUrCrCrGrUrUrArUrCrArAr
CrUrUrGrArArArArArGrUrGrGrCrArCrCrGrArGrUrCrGrGr
UrGrCmU*mU*mU*rU 3′
‘r_’ = RNA bases (e.g. ‘rA′)
‘m_*’ = phosphorothioated 2′-O-methyl RNA bases
e.g. ‘mA*’
ssDNA sequence (IDT; SEQ ID NO: 8):
5′ G*C*CACCTACGGCAAGCTGACCCTGAAGTTCATCTGCACCACCGG
CAAGCTGCCCGTGCCCTGGCCCACCCTCGTGACCACCCTGAGCCACGGC
GTGCAGTGCTTCAGCCGCTACCCCGACCACAT*G*A 3′
‘_*’ = phosphorothioated DNA bases eg. ‘A*’

6.6 Particle Size and Zeta Potential Measurement for pDNA, mRNA, siRNA, Cas9 mRNA/sgRNA and Cas9 mRNA/sgRNA/ssDNA Polyplexes as Well as mRNA LNPs

Polyplexes and LNPs were formulated in HBG as described previously at concentrations of 12.5 μg/mL mRNA (CleanCap® FLuc mRNA (5moU); Trilink Biotechnologies, San Diego, CA, USA), and 10 μg/mL pDNA (pCMVLuc; Plasmid Factory GmbH, Bielefeld, Germany) respectively. siRNA polyplexes were prepared at concentration of 25 μg/mL in a total volume of 80 μL HBG. Polyplexes for Cas9 mRNA/sgRNA experiments were formulated in HBG as described above at concentrations of 5 μg/mL RNA (weight ratio 1:1 of CleanCap® Cas9 mRNA (5moU); Trilink Biotechnologies, San Diego, CA, USA; and sgGFP1; IDT, Coralville, IA, USA) or 12.5 μg/mL RNA (weight ratio 1:1 of CleanCap® Cas9 mRNA (5moU); Trilink Biotechnologies, San Diego, CA, USA; and sgDMDex23; IDT, Coralville, IA, USA), respectively. For HDR experiments, Cas9 mRNA/sgGFP/ssDNA polyplexes were formulated in HBG with 1611 at an N/P of 18 as previously described. Total RNA (Cas9 mRNA and sgGFP) to ssDNA (all IDT, Coralville, IA, USA) weight ratios of 1:0, 1:0.5 and 1:1 resulted in a total nucleic acid concentration of 5 μg/mL. Measurements of size and zeta potential were performed with a Zetasizer Nano ZS (Malvern Instruments, Malvern, Worcestershire, U.K.) in a folded capillary cell (DTS1070) by dynamic and electrophoretic laser-light scattering (DLS, ELS). Size and polydispersity index were measured in 80 μL (siRNA polyplexes, mRNA LNPs) or 100 μL (else) of the above described nanoparticle solutions using the following instrument settings: equilibration time 30 sec, temperature 25° C., refractive index 1.330, viscosity 0.8872 mPa*s. Samples were measured three times with six sub runs per measurement. For measurement of the zeta potential, all samples were diluted to 800 μL with HBG or HEPES (for siRNA polyplexes) directly before measurement. Parameters were identical to the size measurement apart from an equilibration time of 60 sec. Three measurements with 15 sub runs lasting 10 sec each were performed, and zeta potentials were calculated by the Smuchowski equation.

### 6.7 Cell Culture

The murine neuroblastoma cell line Neuro2A (N2a), reporter cell lines for Cas9 experiments (HeLa mCherry-DMDex23-eGFP, and HeLa GFPd2; see below), and N2a/eGFPLuc cells (stably expressing the enhanced green fluorescent protein/luciferase (eGFPLuc) fusion gene) [Schaffert et al. Angew Chem, Int Ed 2011] were cultured in Dulbecco's modified Eagles's medium (DMEM)-low glucose (1 g/L glucose) supplemented with 10% (v/v) fetal bovine serum (FBS), 4 mM of stable glutamine, 100 U/mL of penicillin, and 100 μg/mL of streptomycin. Human hepatocellular carcinoma cell lines Huh7 and HepG2 were cultured in Dulbecco's Modified Eagle Medium (DMEM)/Nutrient Mixture F12-Ham, supplemented with 10% (v/v) FBS, 4 mM of stable glutamine, 100 U/mL of penicillin and 100 μg/mL streptomycin. The human prostate cancer cell line DU145 was cultured in RPMI-1640 medium supplemented with 100 U/mL of penicillin, 100 μg/mL of streptomycin, 10% (v/v) FBS and 4 mM of stable glutamine. The human cervical cancer cell line KB/eGFPLuc as well as DU145/eGFPLuc cells (both cell lines stably expressing eGFPLuc) were also cultured in supplemented RPMI-1640 medium [Dohmen et al. ACS Nano 2012; Frohlich et al. J Control Release 2012]. The cells were cultured at 37° C. and 5% CO₂ in an incubator with a relative humidity of 95%.

6.8 Reporter Cell Lines for Cas9 mRNA/sgRNA Experiments

### Positive Read-Out Reporter Cell Line Used for Cas9/sgDMDex23 Polyplex Transfection Efficiency

A positive read-out reporter system in HeLa cells was used to test the transfection efficiency of Cas9 mRNA/sgDMDex23 polyplexes. An artificial mCherry construct, interrupted by a DMD exon 23 was stably expressed by the HeLa cell line used in this study (HeLa mCherry-DMDEx23-eGFP). This disruption by DMD exon 23 results in a non-functional mCherry protein. Cas9 induced cleavage at the 3′ prime end of the DMD exon 23 mediates expression of functional mCherry protein. The transfection efficiency can be quantified by the percentage of mCherry positive cells measured by flow cytometry as described below.

### Reporter Cell Line for Homologous Directed Repair (HDR)

The homologous directed repair (HDR) efficacy was assessed by the conversion of eGFP to BFP in a HeLa cell line stably expressing destabilized eGFP (HeLa GFPd2 cell line). HDR-mediated repair following the Cas9-induced double strand break (DSB) of the DNA can replace the 66th amino acid tyrosine (code: TAC) in eGFP sequence with histidine (code: CAT) [Farbiak et al. Adv Mater 2021]. In flow cytometric analysis after treatment with Cas9 mRNA/sgGFP/ssDNA-template polyplexes three cell populations were expected: eGFP positive cells representing non-edited cells; eGFP and BFP negative cells indicating gene knock out by non-homologous end joining (NHEJ) of the DSB and BFP positive and eGFP negative cells suggesting HDR-mediated gene correction.

6.9 Cellular Transfection Efficiency of pDNA, mRNA, and siRNA Polyplexes as Well as mRNA LNPs by Luciferase Expression Assay

One day prior to nucleic acid delivery, 10,000 N2a, 10,000 DU145, 8000 Huh7, and 8000 HepG2 cells/well were seeded in 96-well plates. Before the treatment, the cell culture medium was replaced by 80 μL of fresh medium containing 10% (v/v) FBS. Transfection efficiency of polyplexes and LNPs was evaluated for different doses of nucleic acid per well. For mRNA, the volumes of 10, 5, and 2.5 μL of polyplex solution (12.5 μg/mL CleanCap® FLuc mRNA (5moU); Trilink Biotechnologies, San Diego, CA, USA) were added to the corresponding wells in triplicate as well as HBG to reach a final volume of 100 μL per well. For pDNA, volumes of 20, 10, 5, 2.6 and 1.4 μL of polyplex solution (10 μg/mL pCMVLuc; Plasmid Factory GmbH, Bielefeld, Germany) and the corresponding HBG volumes were added in the same way to the wells. In the case of mRNA LNPs, 20 or 5 μL of LNP solution were added per well, containing 250 or 62.5 ng CleanCap® FLuc mRNA (5moU) (Trilink Biotechnologies, San Diego, CA, USA), respectively.

HBG buffer was used as negative control. Succinylated branched polyethylenimine 25 kDa (succinylation degree of 10%, SuccPEI) [Zintchenko et al. Bioconj Chem 2008] at a w/w (weight/weight) ratio of 4 [Krhac Levacic et al. J Control Release 2021] as well as previously published T-Shape lipo-oligoaminoamide 1218 at an N/P ratio of 12 [Krhac Levacic et al. J Control Release 2021] were used as positive controls for mRNA. For pDNA, linear polyethylenimine 22 kDa (LPEI) [Schaffert et al. Pharm Res 2011; Rödl et al. In Nanotechnology for Nucleic Acid Delivery: Methods and Protocols; Ogris, M., Sami, H., Eds.; Springer: New York, NY, 2019] was used at an N/P ratio of 6 as positive control [Russ et al. Gene Ther 2008; Lschelt et al. Nanomedicine 2014; Berger et al. Biomacromolecules 2021]. DLin-MC3-DMA (Patisiran) and SM-102 (Moderna COVID-19 vaccine) at an N/P ratio of 4.5 served as positive controls for mRNA LNPs. After 24 h incubation at 37° C., the medium was removed and cells were treated with 100 μL of cell culture 0.5× lysis buffer (Promega, Mannheim, Germany) and frozen at −80° C. at least overnight. Prior to measurement of luciferase activity, plates were incubated for 1 h at RT. For mRNA experiments (polyplexes, LNPs), the cell lysate was diluted in PBS (phosphate buffered saline; 136.9 mM NaCl, 2.7 mM KCl, 8.1 mM Na₂HPO₄, 1.5 mM KH₂PO₄) at an indicated, suitable ratio (i.e., 1:10 or 1:100) and mixed thoroughly. In exception to this, for mRNA LNP experiments in Huh7 cells at a dose of 62.5 ng mRNA/well, no dilution was done. Luciferase activity in the 35 μL cell lysate was measured by a Centro LB 960 plate reader luminometer (Berthold Technologies, Bad Wildbad, Germany) using LAR buffer (20 mM glycylglycine; 1 mM MgCl₂; 0.1 mM EDTA (ethylenediaminetetraacetic acid); 3.3 mM DTT (dithiothreitol); 0.55 mM ATP (adenosine 5′-triphosphate); 0.27 mM coenzyme A, pH 8-8.5) supplemented with 5% (v/v) of a mixture of 10 mM luciferin and 29 mM glycylglycine. Transfection efficiency was calculated for 10,000 or 8000 cells depending on the seeded amount and presented as relative light units (RLU) per well. For siRNA, an initial number of 5000 cells/well were plated in 96-well plates. The volumes of 2.5, 1.3 and 0.6 μL (25 μg/mL siRNA) of polyplex solution as well as corresponding volumes of HBG (final volume of 100 μL per well) were applied to the cells for 48 h with the same transfection protocol of mRNA and pDNA polyplexes. SuccPEI (w/w 4) [Zintchenko et al. Bioconj Chem 2008] and 1214 (N/P 12) [Luo et al. J Control Release 2020] were considered as positive controls. The cell lysate was subjected to the measurement after 45 min incubation of the lysis buffer-treated cells at RT. RLU was presented as percentage of the luciferase gene expression of the HBG buffer-treated cells.

6.10 Co-Delivery Efficiency of mRNA/pDNA Polyplexes by Flow Cytometry

mRNA/pDNA polyplex treatments were performed in triplicate in 96-well plates. N2a cells were seeded 24 h prior to transfection (10,000 cells/well). On the next day, the medium was replaced with 80 μL of fresh pre-warmed medium containing 10% (v/v) FBS. The nanoparticles were prepared as described above, and the volumes of 10 and 5 μL of polyplex solution (2.5 μg/mL mCherry mRNA and 2.5 μg/mL pEGFP-N1 pDNA) were added. 20 μL HBG buffer per well was used as negative control. After 24 h of treatment, cells were collected and re-suspended in PBS solution containing 10% (v/v) FBS (FACS buffer) for flow cytometric evaluation. All samples were analyzed by flow cytometry using a CytoFLEX S Flow Cytometer (Beckman Coulter, Brea, CA, USA). Shortly before the measurement, 1 ng/μL 4′, 6-diamidino-2-phenylindole (DAPI) was added and used to discriminate between viable and dead cells. The cellular fluorescence was assayed by excitation of DAPI at 405 nm and detection of emission at 450 nm. Only isolated viable cells were evaluated. Gates were set compared to control measurements with HBG buffer-treated cells. The percentage of mCherry and eGFP positive cells represented efficient mRNA and pDNA transfection, respectively. Cells shifted to the mCherry and GFP positive gate indicated successful co-delivery and expression of both, mRNA and pDNA in one cell. Flow cytometry data were analyzed using FlowJo™ v10.8 flow cytometric analysis software by FlowJo, LLC (Becton, Dickinson and Company, U.S.). The cellular fluorescence expression was assayed by excitation at 561 nm and 488 nm, the detection of emission at 610 nm and 530 nm for mCherry and eGFP, respectively.

6.11 Cellular eGFP Knock Out Efficiency of Cas9 mRNA/sgRNA Polyplexes by Flow Cytometry

Cas9 mRNA/sgRNA polyplex treatments were performed in triplicate in 96-well plates. N2a eGPFLuc cells were seeded 24 h prior to transfection (5000 cells/well). On the next day, the medium was replaced with 80 μL of fresh pre-warmed medium containing 10% (v/v) FBS. The nanoparticles were prepared as described above, and the volumes of 20, 10, and 5 μL of polyplex solution (2.5 μg/mL Cas9 mRNA and 2.5 μg/mL sgGFP1) were added. 20 μL HBG buffer per well was used as negative control. After 24 h of treatment, the cells were expanded, and the medium was replaced by 100 μL of fresh pre-warmed medium and incubated for additional 48 h. Flow cytometric evaluation and analysis was performed as described in section “Co-delivery efficiency of mRNA/pDNA polyplexes by flow cytometry”. The cellular eGFP expression was assayed by excitation at 488 nm and the detection of emission at 530 nm. Only isolated viable cells were evaluated. The eGFP knock out efficiency was determined as the percentage of eGFP negative cells normalized to the control measurements with HBG buffer-treated cells.

6.12 Cellular Transfection Efficiency of Cas9 mRNA/sgDMDex23 Polyplexes and RNP Polyplexes by Flow Cytometry

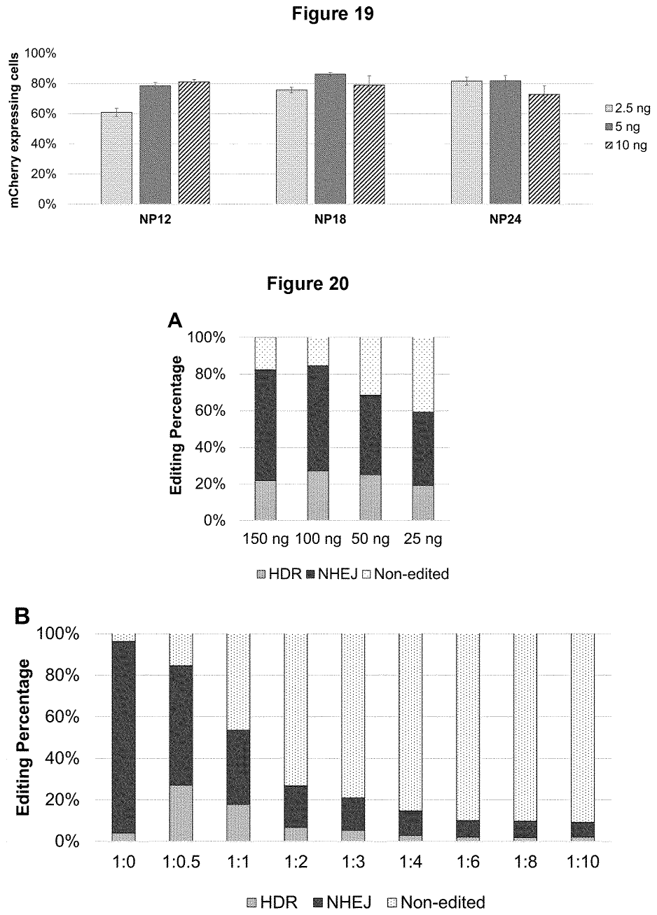

HeLa mCherry-DMDex23-eGFP cells were seeded 24 h prior to transfection (5000 cells/well). On the next day, the medium was replaced with 80 μL of fresh pre-warmed medium containing 10% (v/v) FBS. Polyplex treatments were performed in triplicate in 96-well plates. The Cas9 mRNA/sgDMDex23 polyplexes were prepared as described above at indicated total RNA concentrations of 5 μg/mL, 6 μg/mL, 12 μg/mL or 12.5 μg/mL total RNA (mRNA/sgRNA weight ratio 1:1). Defined dilutions of polyplex solutions, representing 2.5 ng, 5 ng, 10 ng, 25 ng total RNA (mRNA/sgRNA weight ratio 1:1) per well (FIG. 18 and FIG. 19), or 8 ng (2.5 nM), 16 ng (5 mM) and 32 ng (10 nM) sgRNA per well (FIG. 21) were pipetted to the corresponding wells. To assess the efficiency in high serum of carrier 1621 (FIG. 19) the polyplexes were formed at a total RNA concentration of 12.5 μg/mL (mRNA/sgRNA weight ratio 1:1), subsequently 10-fold diluted with 100% FBS and incubated for 2 h at 37° C. Afterwards, volumes representing 2.5 ng, 5 ng and 10 ng total RNA (Cas9mRNA/sgRNA weight ratio 1:1) were added to the corresponding wells. HBG buffer was added to reach a final volume of 100 μL per well in all experiments.

For testing the efficiency of RNP containing polyplexes, nanoparticles were formulated as described above at a RNP stock concentration of 375 nM (Cas9 protein and sgDMDex at molar ratio 1:1). Volumes resulting in 2.5 nM, 5 nM, and 10 nM RNP (8, 16, 32 ng sgRNA/well) were added to the corresponding wells. HBG buffer was added to reach a final volume of 100 μL per well in all experiments.

Addition of 20 μL HBG buffer was used as negative control for both Cas9 mRNA/sgDMDex23 and Cas9 protein and sgDMDex polyplexes. The cells were incubated for 24 h. Afterwards, the cells were trypsinized, expanded and incubated for additional 2 days. Flow cytometric evaluation and analysis was performed as described in section “Co-delivery efficiency of mRNA/pDNA polyplexes by flow cytometry”. The cellular mCherry expression was assayed by excitation at 561 nm and the detection of emission at 610 nm. Only isolated viable cells were evaluated. The transfection efficiency was determined as the percentage of mCherry positive cells.

6.13 eGFP to BFP Conversion (HDR) Evaluation by Flow Cytometry

Treatments with polyplexes co-formulating Cas9 mRNA, sgGFP and ssDNA were performed in triplicate in 96-well plates. HeLa GFPd2 cells were seeded 24 h prior to transfection (5000 cells/well). On the next day, the medium was replaced with 80 μL of fresh pre-warmed medium containing 10% (v/v) FBS. The nanoparticles were prepared as described above. 20, 13.3, 6.7, and 3.3 μL of polyplex solution were added to the corresponding wells. HBG buffer was added to reach a final volume of 100 μL per well. Addition of 20 μL HBG buffer was used as negative control. The cells were incubated for 48 h. Afterwards, the cells were trypsinized, expanded and incubated for an additional 3 days. Then, the cells were harvested re-suspended in PBS solution containing 10% (v/v) FBS (FACS buffer). By flow cytometric evaluation the loss of GFP indicated gene knock out mediated NHEJ; BFP expression represented conversion from the GFP reporter protein to BFP by homology-directed repair while GFP expressing cells show non-edited reporter cells. BFP expression was assayed by excitation at 405 nm and the detection of emission at 450 nm. GFP fluorescent was detected with excitation at 488 nm and 530 nm emission.

6.14 Luciferase Expression of mRNA Polyplexes and mRNA LNPs In Vivo in Tumor-Bearing Mice

In vivo experiments were performed according to the guidelines of the German Animal Welfare Act and were approved by the animal experiments ethical committee of the Government of Upper Bavaria (accreditation number Gz. ROB-55.2-2532.Vet_02-19-20). Neuro2a cells (10⁶ cells/150 μL PBS) were inoculated subcutaneously into the left flank of 6-week-old female A/J mice (Envigo RMS GmbH, Dusseldorf, Germany). Mice were randomly divided into groups of three (in the case of mRNA LNPs) or five (in the case of mRNA polyplexes) and were housed in isolated ventilated cages under specific pathogen free conditions with a 12 h day/night interval, and food and water ad libitum. Weight and general well-being were monitored continuously. Tumor size was measured with a caliper and determined by formula: a*b²/2 (a=longest side of the tumor; b=widest side vertical to a). When tumors reached a size of 250-500 mm3, the experiments were performed by intravenous tail vein injection of polyplexes or LNPs formed as described in 6.1 and 6.3, containing 1 μg, 3 μg or 10 μg of stabilized firefly luciferase encoding mRNA (CleanCap™ FLuc mRNA (5moU); Trilink Biotechnologies, San Diego, CA, USA) in 150 μL HBG. Polyplexes containing 10 μg mRNA were dialyzed for 1 h at 4° C. against HBG (dialysis membrane MWCO 100-500 Da; Spectra/Por®, Carl Roth, Karlsruhe, Germany) prior to application to remove small amounts of ethanol. In the case of mRNA polyplexes, mice were euthanized either 6 h or 24 h after injection. The organs (tumor, lungs, liver, kidneys, spleen, brain, heart, muscle) were dissected and washed carefully with PBS, followed by analysis via ex vivo luciferase gene expression assay. In the case of mRNA LNP experiments, mice were euthanized 20 h post-injection and the organs (lungs, liver, spleen, kidneys, heart, muscles, and tumor) were dissected and washed with PBS. The luciferase gene expression was determined as described above and is presented as relative light units (RLU) per gram (g) tumor/organ.

### 6.15 Ex Vivo Luciferase Gene Expression Assay of Organs, Muscles and Tumors

To evaluate luciferase mRNA delivery by an ex vivo luciferase gene expression assay, organ and tumor tissues were homogenized in Luciferase Cell Culture Lysis Reagent 1× (Promega, Mannheim, Germany), supplemented with 1% (v/v) protease and phosphatase inhibitor cocktail (Sigma-Aldrich, Munich, Germany) using a tissue and cell homogenizer (FastPrep®-24, MP Biomedicals, USA). Then, the samples were frozen overnight at −80° C. to ensure full lysis. In a next step, the samples were thawed and centrifuged for 10 min at maximum speed (˜13,000 rpm) and 4° C. Luciferase activity in 25 μL supernatant was measured in a Centro LB 960 plate reader luminometer (Berthold Technologies, Bad Wildbad, Germany) for 10 sec after addition of 100 μL/well of a LAR buffer solution (20 mM glycylglycine, 1.0 mM MgCl₂, 0.10 mM EDTA, 3.3 mM DTT, 0.55 mM ATP, and 0.27 mM coenzyme A; pH 8-8.5) supplemented with 5% (v/v) of a mixture of 10 mM luciferin and 29 mM glycylglycine.

### 6.16 CellTiterGlo Assay

HeLa cells were seeded 24 h prior to transfection (5000 cells/well) in 96-well plates. Immediately prior to transfection, the medium was replaced with suitable amounts of fresh medium containing 10% (v/v) FBS to reach a final volume of 100 μL during transfection. The nanoparticles were prepared as described in section 6.1 and transfected with indicated doses. The medium was removed at 24 h after transfection, and 25 μL of fresh, pre-warmed medium as well as 25 μL of CellTiterGlo® Reagent (Promega, Mannheim, Germany) were added to each well [S. Berger et al., Biomacromolecules 2021, 22, 1282; A. Krhač Levačić et al., J Controlled Release 2021, 339, 27]. After incubation on an orbital shaker for 30 min at RT, luminescence was recorded using a Centro LB 960 plate reader luminometer (Berthold Technologies, Bad Wildbad, Germany). The luminescent signals (in RLU) of the samples were normalized to the luminescent signal of the negative control (HBG buffer-treated control cells). Results were presented as relative metabolic activity related to the negative control. Experiments were performed as triplicates.

### 6.17 Annexin/PI Assay

Polyplex treatments were performed in duplicates in 24-well plates. HeLa cells were seeded 24 h prior to transfection (40,000 cells/well). Immediately prior to transfection, the medium was replaced with 475 μL of fresh medium containing 10% FBS for pDNA polyplexes and with 487.5 μl for mRNA polyplexes. The nanoparticles were prepared as described in section 6.1. Transfection was performed with 12.5 μL of polyplexes containing 125 ng/well pDNA or 6.3 μL polyplex solution containing 78 ng/well mRNA, respectively. HBG was added to reach a total volume of 500 μl per well, i.e. 12.5 μl HBG for pDNA and 6.2 μl HBG for mRNA. As negative control, 25 μl HBG for pDNA and 12.5 μl HBG for mRNA were used, respectively. After 24 h of incubation, the cells were collected and incubated with annexin V incubation reagent (prepared according to manufacturer's protocol of Bio-Techne GmbH, Wiesbaden, Germany) for 10 min before flow cytometer analysis using a CytoFLEX S Flow Cytometer (Beckman Coulter, Brea, CA, USA). Gates were set compared to control measurements with HBG-buffer treated cells and with exclusion of debris cells. Annexin V and propidium iodide negative cells were considered as healthy cells. Cells shifted to the Annexin V and propidium iodide positive gate indicated late apoptotic cells, whereas cells shifted to only Annexin V-positive gate were considered as early apoptotic, and PI-positive, but Annexin-V-negative cells were not viable. Flow cytometry data were analyzed using FlowJo™ v10.8 flow cytometric analysis software by FlowJo, LLC (Becton, 20 Dickinson and Company, U.S.).

### 6.18 Formation of Poly(Inosine:Cytosine) Polyplexes.

Cytotoxic double-stranded RNA poly(inosine:cytosine), poly(I:C), was formulated at a concentration of 12.5 μg/mL in HBG with the carriers 1611 (at N/P 18), 1719 (at N/P 12), and 1752 (at N/P 24) by mixing as described in section 6.1.

6.19 Formation of Shielded and Targeted pDNA Polyplexes.

Synthesis of targeting lipidic anchor DSPE-PEG70-GE11 by copper free azide-DBCO orthogonal click reaction. Equal molar amounts of azide-modified PEG-lipid (DSPE-PEG2k-N3) and DBCO-PEG-functionalized peptide GE11 ligand (DBCO-PEG₂₄-GE11) [Truebenbach et al, Int. J. Pharm. 2019, 569, 118570] were mixed together in HBG buffer (20 mM HEPES, 5% (w/v) glucose; pH 7.4) and were incubated overnight at RT under constant shaking (250 rpm) in order to generate lipid-PEG-ligand conjugate (DSPE-PEG70-GE11 conjugate).

Formation of shielded and targeted pDNA polyplexes. All shielded and targeted pDNA polyplexes were formed at an N/P ratio of 12, containing three lipidic components, namely, the LAF-Stp carrier (Table C), DSPE-PEG2k-N₃, and DSPE-PEG70-GE11 conjugate. The molar amount of LAF carrier needed to create N/P=12 was set as 75% of total molar amount of lipids, whereas DSPE-PEG2k-N3 and DSPE-PEG70-GE11 conjugate were set at 12.5% each. In the case of shielded control polyplexes, DSPE-PEG2k-N3 was used at 25%. Prior to nanoparticle formation, the required molar amounts of the lipidic components were mixed together in H₂O; pDNA was diluted in HBG buffer. Nanoparticle formation itself was performed by mixing (rapid pipetting) equal volumes of carrier and nucleic acid, followed by incubation for 40 min at RT.

TABLE C
LAF carriers used for shielded and targeted pDNA polyplexes

| ID | Code | Topology | m | n | Sequence (N −> C) |
|---|---|---|---|---|---|
| 1719 | 12Oc-U1-2:4 | U1 | 2 | 2 | [K(12Oc)]2-Stp2-[K(12Oc)]2 |
| 1730 | 8Oc-B2-2:4 | B2 | 2 | | K[K[8Oc)2]2-Stp2 |

### 7. In Vivo Applications

7.1 Intramuscular Application of Luciferase mRNA Polyplexes and 1621 LNPs in Tumor-Free Mice

In vivo experiments were performed according to the guidelines of the German Animal Welfare Act and were approved by the animal experiments ethical committee of the Government of Upper Bavaria (accreditation number Gz. ROB-55.2-2532.Vet_02-19-19). 6-weeks-old female BALB/c mice were randomly divided into groups of four and were housed in isolated ventilated cages under specific pathogen-free conditions with a 12 h day/night interval, and food and water ad libitum. Weight and general well-being were monitored continuously. The experiments were performed after subcutaneous treatment with Carprofen (5 mg/kg) by intramuscular injection (musculus biceps femoris) of polyplexes and LNPs, containing 3 μg of stabilized firefly luciferase encoding mRNA (CleanCap™ FLuc mRNA (5moU); Trilink Biotechnologies, San Diego, CA, USA) in 50 μL HBG. Mice were euthanized 6 h after injection. Injected muscles and complementary none-injected muscles were dissected and washed carefully with PBS, followed by analysis via ex vivo luciferase gene expression assay as described in section 6.15. Luciferase activity is presented as RLU/g injected muscle.

### II. Results, Figures & Tables

### Example 1: PDNA Polyplexes

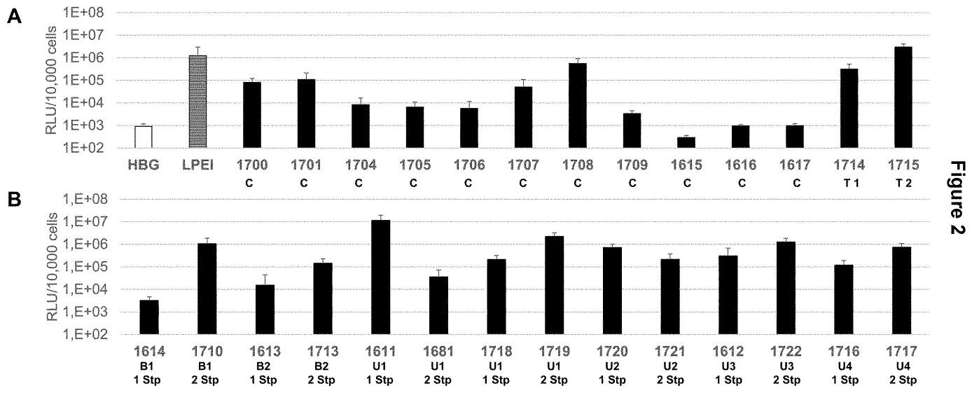

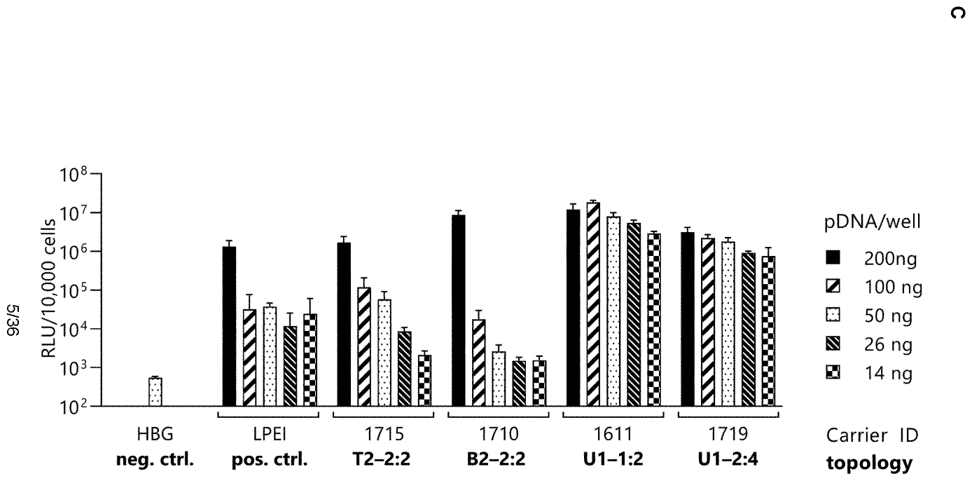

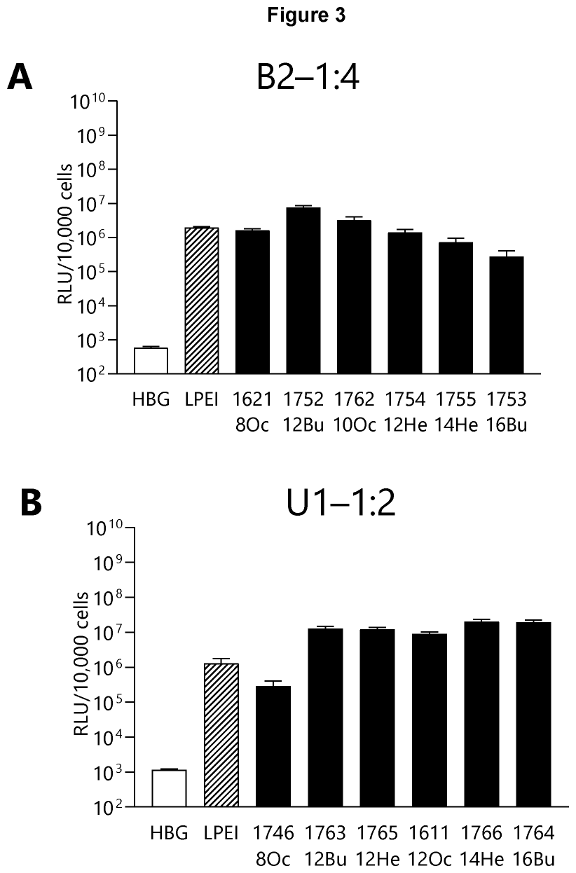

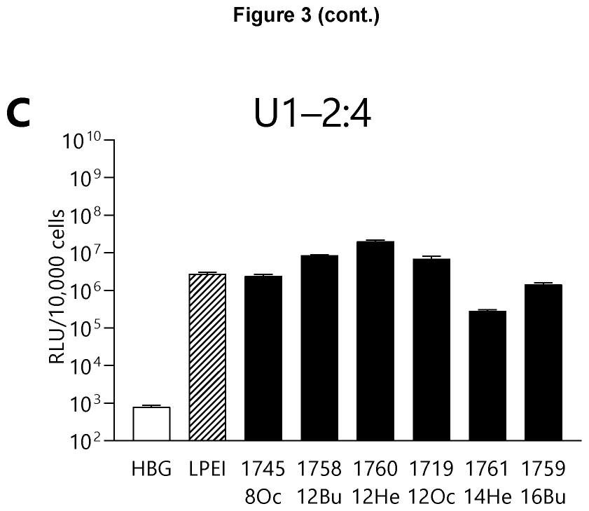

Complexes formed by the novel LAF carriers comprising 12Oc and Stp with pDNA were characterized and all 12Oc carriers that formed polyplexes were screened in pDNA transfection using the luciferase expression assay. The novel LAF carriers were able to form defined, homogenous pDNA complexes with positive zeta potential (Tab. 4). In contrast to the PCD:ACD ratio used in the description, which defined the smallest ratio if not otherwise stated, the Stp/LAF ratio referred to in the Examples 1 to 8 describes the Stp:LAF content, i.e., an Stp/LAF ratio of 1:2 contains 1 Stp and 2 LAF, an Stp/LAF ratio of 2:4 contains 2 Stp and 4 LAF, an Stp/LAF ratio of 2:2 contains 2 Stp and 2 LAF etc. The screening in N2a cells of the different 12Oc carriers with different topologies (FIGS. 2 A and B) revealed similar transfection efficiency like gold standard LPEI at standard pDNA dose (200 ng/well) for comb structure 1708, T-shape structures 1714 and 1715, and B1 structure 1710. All these carriers have a Stp/LAF ratio of 2:2 in common. However, U-shape structures turned out to be the most potent pDNA carriers, especially those with a Stp/LAF ratio of 1:2 (1611) and 2:4 (1719, 1722, 1717). U1 structure 1611 was the best 12Oc performer and outperformed LPEI by 10-fold at the highest and by more than 100-fold at the lowest tested pDNA dose. At lower pDNA doses per well, the high effectiveness of the U1 carriers 1611 and 1719 could be further demonstrated, as there was only a minor decrease in transfection efficiency even at one-eighth of the pDNA dose (FIG. 2C). Carriers of other topologies (T2, 1715; B1, 1710) as well as LPEI showed good performance only at the highest dose and a strong dose-dependent efficiency. Comparing different LAFs in analogous structures with U1 and B2 topologies (FIG. 3A-C), revealed that the alkyl chain length was more important than the length of the amino fatty acid. An alkyl chain length of 12 seemed to be beneficial for transfection efficiency (U1—12Bu, 12He, 12Oc; B2—12Bu).

TABLE 4
Particle size and zeta potential of pDNA polyplexes - selected examples.

| | 1715 | 1710 | 1611 | 1719 |
|---|---|---|---|---|
| Z-average in nm | 89.8 ± 1.4 | 111.8 ± 11.4 | 215.5 ± 2.5 | 110.4 ± 1.89 |
| Pdl | 0.100 ± 0.010 | 0.246 ± 0.024 | 0.139 ± 0.027 | 0.220 ± 0.001 |
| Zeta potential in mV | 24.47 ± 1.4 | 15.6 ± 2.1 | 24.4 ± 0.9 | 13.0 ± 0.5 |

Comparison of different LAF containing carriers formulated as pDNA polyplexes at N/P 12 and a concentration of 10 μg/mL pCMVLuc. Data presented as average of three sub-runs ± SD.

### Example 2: mRNA Polyplexes

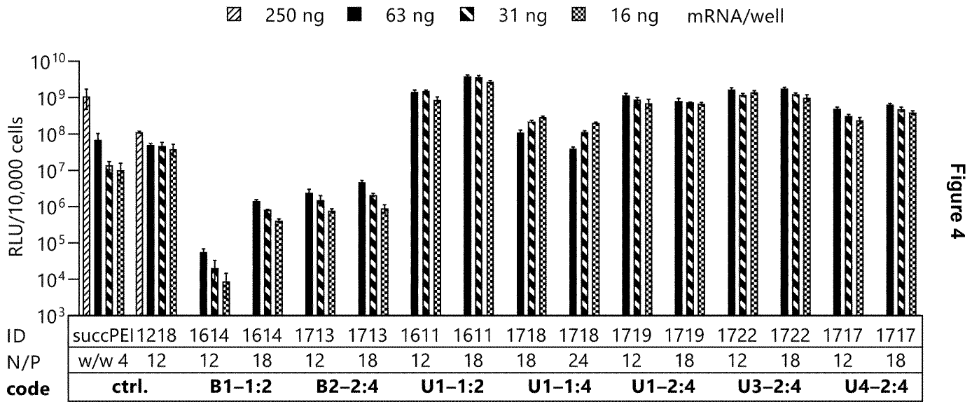

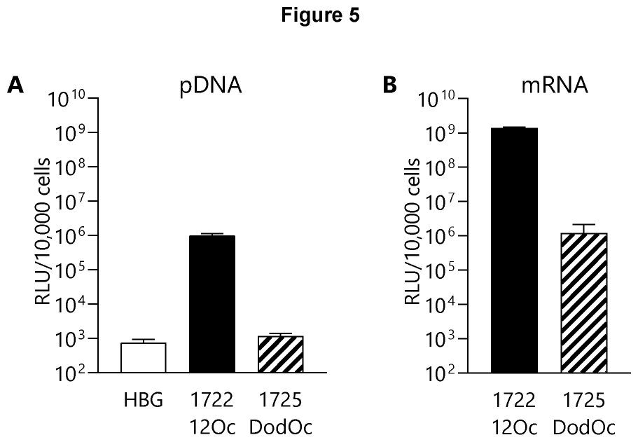

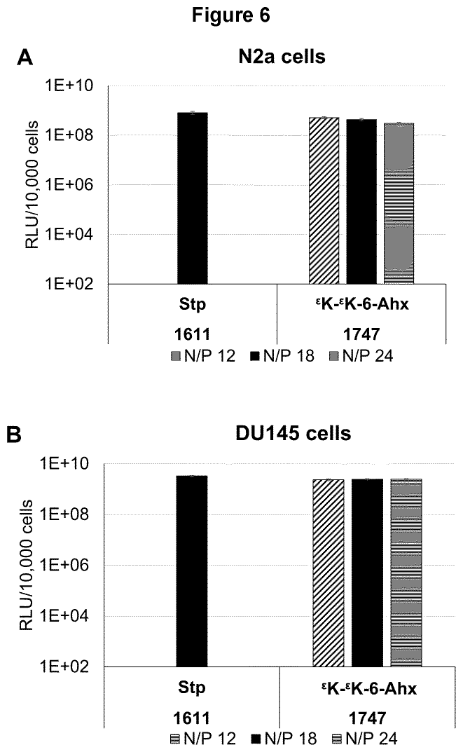

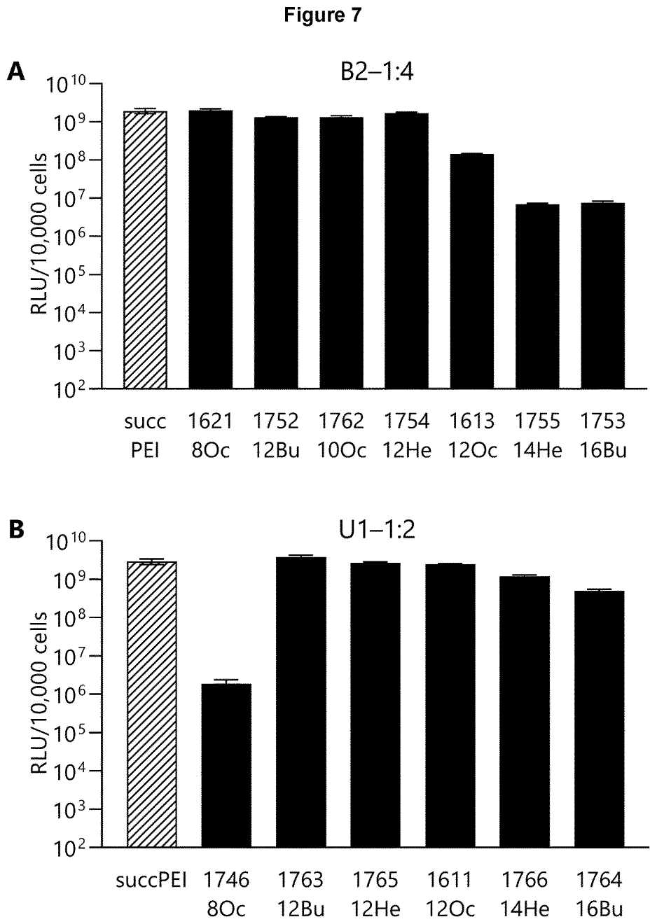

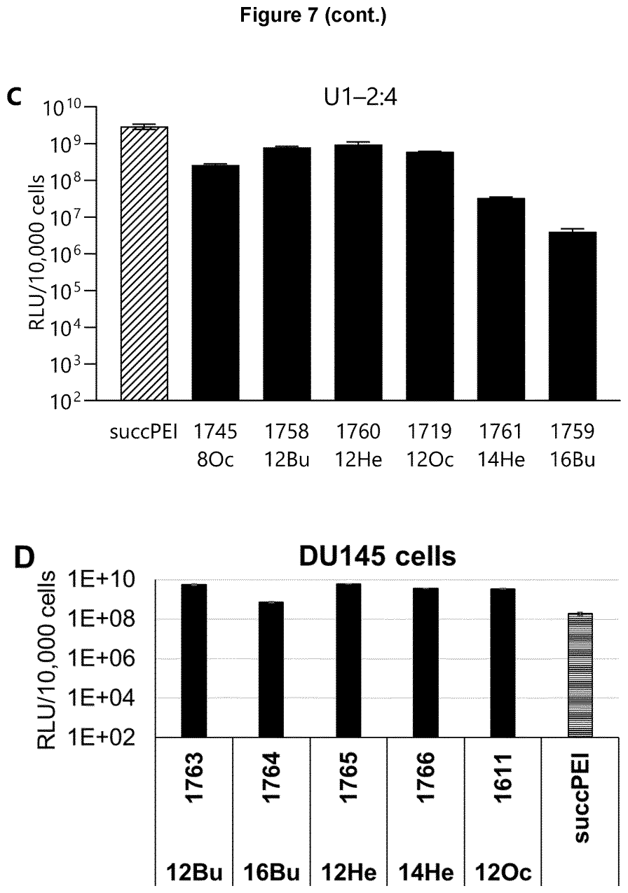

Complexes formed by the novel LAF carriers with mRNA were characterized and transfections efficiencies of 12Oc carriers with all different topologies were compared and analyzed using the luciferase expression assay. The novel LAF carriers were able to form defined, homogenous mRNA complexes with positive zeta potential, both at in vitro and in vivo concentration (Tab. 5, 6). High luciferase expression levels in N2a cells were observed for different LAF carriers, even at one-sixteenth of the standard mRNA dose (250 ng/well) (FIG. 4). Especially, carriers with U-shape topology (1611—U1, Stp/LAF=1:2; 1719—U1, Stp/LAF=2:4; 1722—U3, Stp/LAF=2:4) showed dose-independent transfection efficiency, outperforming positive controls succPEI and 1218 by far. The benefit and need of the protonable tertiary amine within the apolar domain of the LAF carriers was confirmed for both pDNA and mRNA polyplexes (FIG. 5). Carrier 1725 containing the control motif DodOc (amide) was ineffective in N2a cells compared to its LAF analog 1722. However, replacing the polar protonable domain Stp by the ^{ε}K-^{ε}K-6-Ahx motif was possible without loss of transfection efficiency (FIG. 6), giving the opportunity for further tuning and optimizing of new carriers. Variation of the LAF within the same structure, such as B2-1613 (FIG. 7A), U1-1611 (FIGS. 7B and D) and U1-1719 (FIG. 7C) indicated that longer alkyl chain lengths (tetradecyl, hexadecyl) might be less favourable for transfection efficiency. This was particularly the case for 1613 and 1719 analogs (FIGS. 7A and C). In the case of B2 carriers, shorter LAFs, especially 8Oc and 12 Bu, were beneficial in terms of particle formation and transfection efficiency. As found for pDNA, the different amino fatty acids seemed to have less impact. Finally, mRNA polyplexes formed with U-shape structures 1611 (12Oc-U1-1:2), 1719 (12Oc-U1-2:4), and 1760 (12He-U1-2:4) exhibited great in vivo performance in N2a tumor-bearing mice with high luciferase expression levels especially in tumors, lungs, liver, and spleen (FIG. 8A+B). Considering both high efficiency and good tolerability, U-shape carrier 1611 turned out to be the most promising candidate for in vivo over a broad range of mRNA doses, mediating high RLU values already at an early time-point of 6 h after injection. Bundles 1621 (8Oc-B2-1:4) and 1752 (12Bu-B2-1:4) were identified to be also highly potent in vivo, yet also very toxic. However, with lowering the dose of 1752 to 1 μg mRNA/animal, toxicity could be handled. Noteworthy, 1752 showed very encouraging results at this very low dose (FIG. 8C).

TABLE 5
Particle size and zeta potential of mRNA polyplexes - selected examples.

| | | | | | | | 1747 |
|---|---|---|---|---|---|---|---|
| | 1611 | 1719 | 1722 | 1732 | 1614 | 1621 | N/P |
| | N/P 12 | N/P 12 | N/P 12 | N/P 12 | N/P 24 | N/P 18 | 18 |
| Z-average in | 103.7 ± | 75.0 ± | 92.5 ± | 98.2 ± | 111.6 ± | 283.1 ± | 91.1 ± |
| nm | 2.5 | 1.3 | 1.4 | 1.3 | 1.4 | 4.4 | 1.3 |
| Pdl | 0.303 ± | 0.225 ± | 0.339 ± | 0.304 ± | 0.223 ± | 0.201 ± | 0.146 ± |
| | 0.020 | 0.020 | 0.024 | 0.010 | 0.017 | 0.012 | 0.013 |
| Zeta potential | 26.0 ± | 33.0 ± | 44.4 ± | 26.2 ± | 15.7 ± | 20.8 ± | 17.5 ± |
| in mV | 1.7 | 1.2 | 2.4 | 1.1 | 1.2 | 0.3 | 0.8 |

Comparison of different LAF containing carriers formulated as mRNA polyplexes at indicated N/P ratios and a concentration of 12.5 μg/mL of CleanCap ® FLuc mRNA (5moU).
Data presented as average of three sub-runs ± SD.

TABLE 6
Particle size and zeta potential of dialyzed
mRNA polyplexes for in vivo application.

| | 1611 | 1719 |
|---|---|---|
| | N/P 18 | N/P 12 |
| Z-average in nm | 150.0 ± 1.0 | 108.7 ± 2.0 |
| Pdl | 0.247 ± 0.033 | 0.176 ± 0.016 |
| Zeta potential in mV | 27.6 ± 1.6 | 30.7 ± 1.7 |

DLS and ELS measurement of mRNA polyplexes formed with carrier 1611 at N/P 18 and 1719 at N/P 12 at the in vivo concentration of 66.7 μg/mL of CleanCap ® FLuc mRNA (5 moU). Data presented as average of three sub-runs ± SD.

### Example 3: MRNA/pDNA Co-Delivery

Co-delivery efficiency of mRNA/pDNA polyplexes with different LAF carriers was analyzed. The carriers were selected based on the existing information from the screening of pDNA and mRNA polyplexes. mCherry mRNA/eGFP pDNA co-expression in N2a cells, even at low combined nucleic acid doses (25 and 50 ng/well), was recognized for various LAF carriers (FIG. 9). Among the tested LAF carriers, most potent structures were carriers with U-shape topology, namely 1611 (U1, 12Oc, Stp/LAF=1:2) and its LAF analogs 1763 (12Bu) and 1765 (12He) as well as 1719 (U1, 12Oc, Stp/LAF=2:4) and 1716 (U4, 12Oc, Stp/LAF=1:4).

### Example 4: siRNA Polyplexes

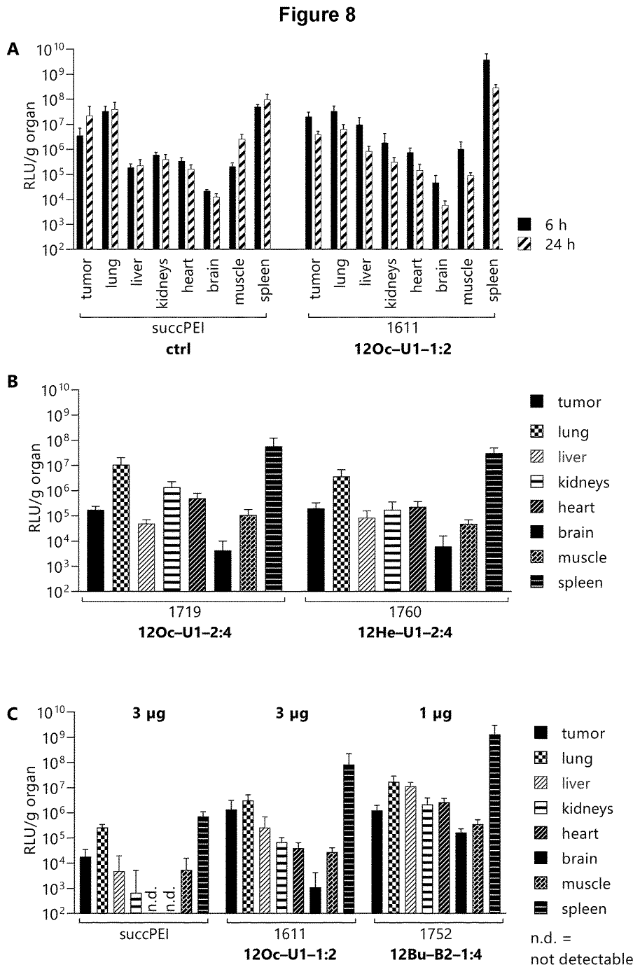

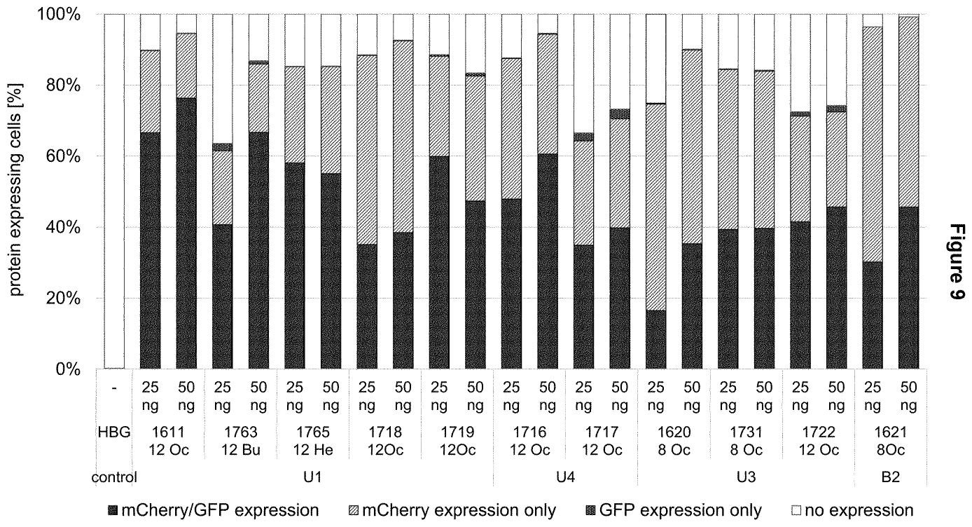

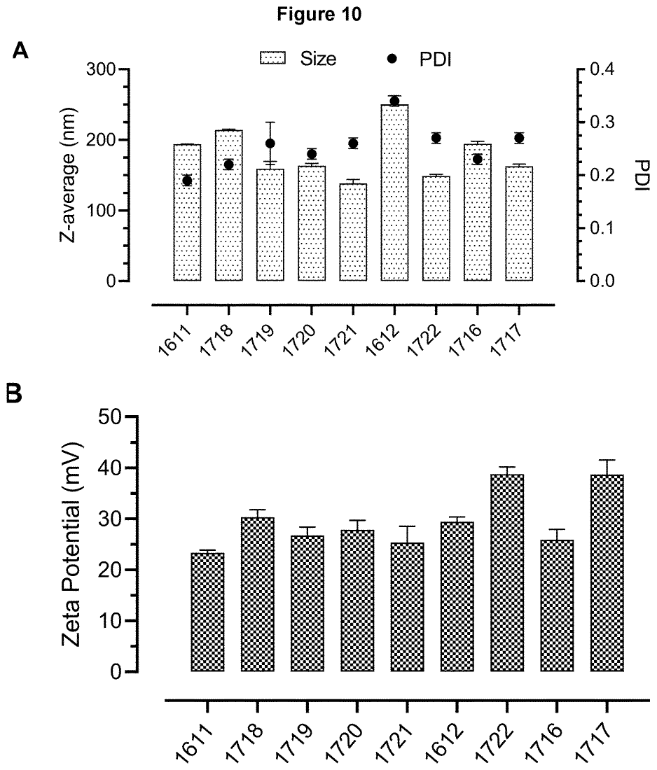

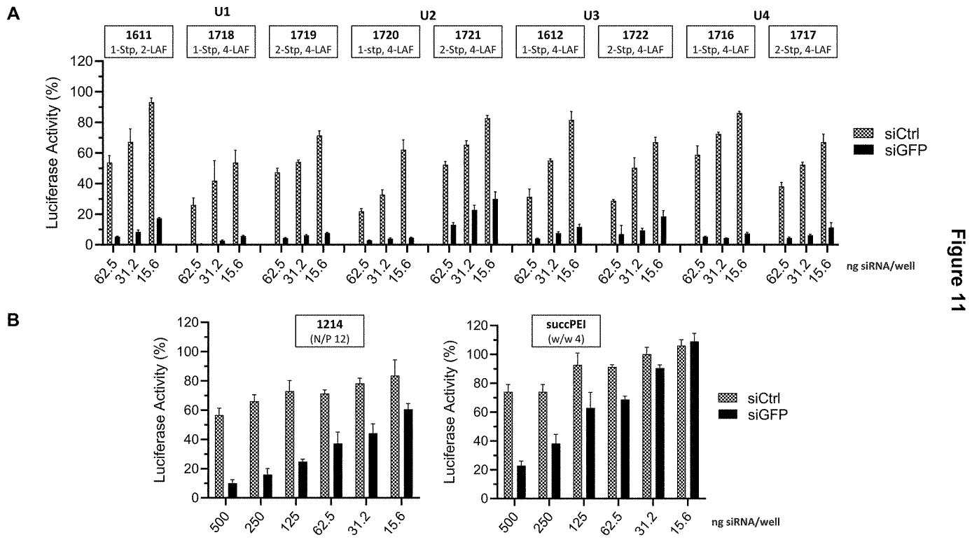

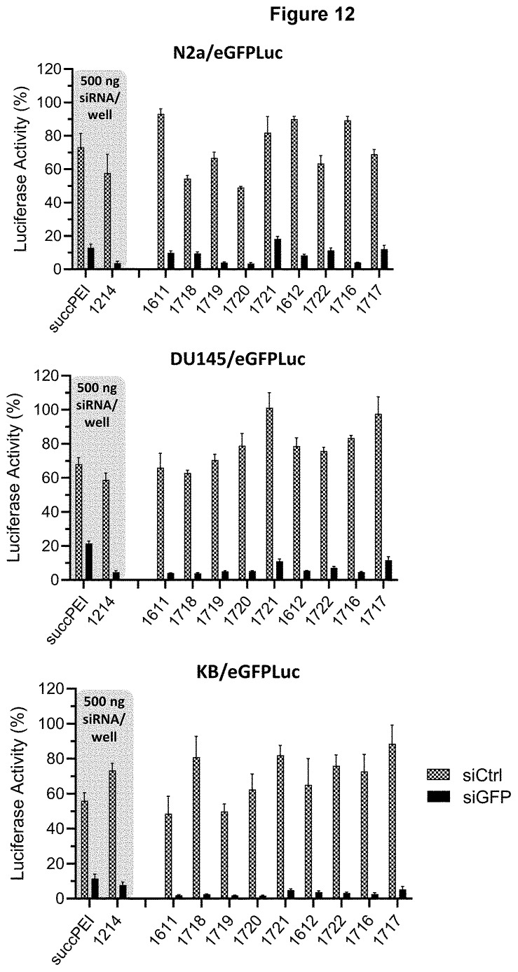

Complexes formed by the novel LAF carriers with siRNA were characterized and transfections efficiencies of 12Oc carriers with different topologies were compared and analyzed using the luciferase expression assay. Only LAF carriers with U-shape topology showed encouraging results. These carriers were able to form defined and homogenous siRNA complexes with positive zeta potential (FIG. 10). They mediated promising gene silencing efficiency in N2a/eGFPLuc cells at low siRNA doses (62.5, 31.2, and 15.6 ng/well) (FIG. 11A). In contrast, in the case of positive control carriers succPEI and 1214, 4- to 8-fold higher siRNA doses were required for sufficient gene silencing (FIG. 11B). For some LAF carriers, unspecific reporter silencing by siCtrl formulations were recognized at high dose. However, by lowering the siRNA dose, the unspecific effects could be solved without reducing the specific siGFP-mediated gene silencing. The high efficiency at low siRNA dose could be confirmed in two additional cell lines (DU145/eGFPLuc, KB/eGFPLuc; FIG. 12). Most potent LAF carrier was 1716 (U4, 12Oc, Stp/LAF=1:4), showing great performance in all three tested cell lines.

To sum up the findings of the physicochemical characterization of polyplexes, distinct LAF carriers could be identified for effective complexation of the three examined cargos pDNA, mRNA, and siRNA. For all three nucleic acids, the U-shape topology seemed to be most promising, whereas an aggregation tendency was observed for the sterically more advanced bundle structures. In the case of bulky carriers like B2 bundles, shorter LAF 8Oc was more beneficial for polyplex formation than longer LAFs such as 12Oc. In general, a Stp/LAF ratio of 1:4 was less suitable for nanoparticle formation, especially for pDNA and mRNA polyplexes. This was the case for all investigated topologies, i.e., combs (1616), bundles (1613) and U-shapes (U1—1718; U2—1720; U3—1612; U4—1716). For many LAF carriers, an N/P ratio of 6 was not enough to sufficiently form defined polyplexes. However, increasing the N/P ratio often led to homogenous particle formation with N/P 12 and 18 turning out to be most suitable for most of the LAF carriers. U-Shapes of the Stp1-series needed higher N/P ratios than those with 2 Stp units to be able to form stable polyplexes. The most reasonable N/P ratios for the distinct LAF carriers and nucleic acids were chosen for the subsequent biological evaluation.

Also in the biological evaluation distinct, but partially overlapping LAF carriers could be identified to be effective for the three examined cargos pDNA, mRNA, and siRNA.

For pDNA polyplexes, U1 structures 1611 (12Oc, Stp/LAF=1:2) and 1719 (12Oc, Stp/LAF=2:4) as well as their 12Bu and 12He analogs were identified as most potent carriers. Also B2 bundle 1752 (12Bu; Stp/LAF=1:4) showed promising activity.

For mRNA polyplexes, U1 carrier 1611 (12Oc, Stp/LAF=1:2) turned out to be the most promising candidate, especially in terms of in vivo application. Furthermore, B2 bundles (Stp/LAF=1:4) 1621 (8Oc) and 1752 (12Bu) were very potent but also toxic. Dose reduction could overcome the toxicity issue at still remained high efficacy.

For siRNA polyplexes, U-shapes were highly effective at very low siRNA doses. U4 carrier 1716 (12Oc, Stp/LAF=1:4) was the best performer in three different cell lines.

### Example 5: mRNA LNPs

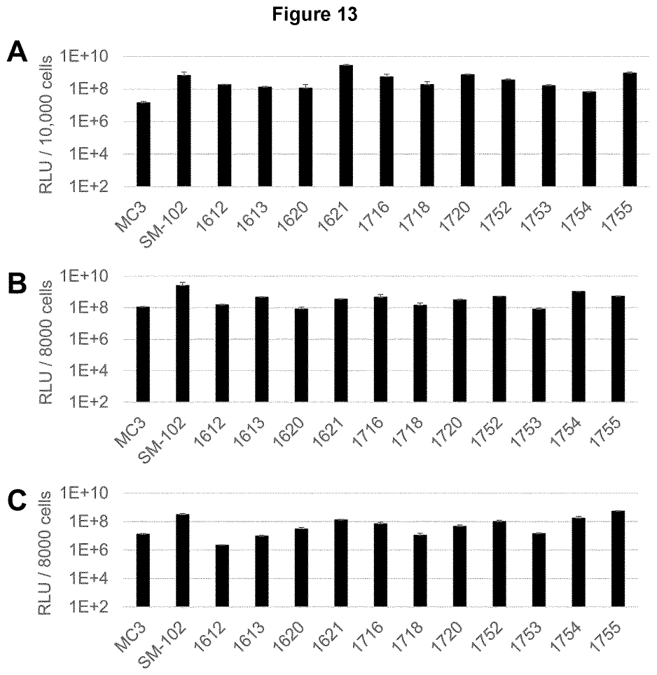

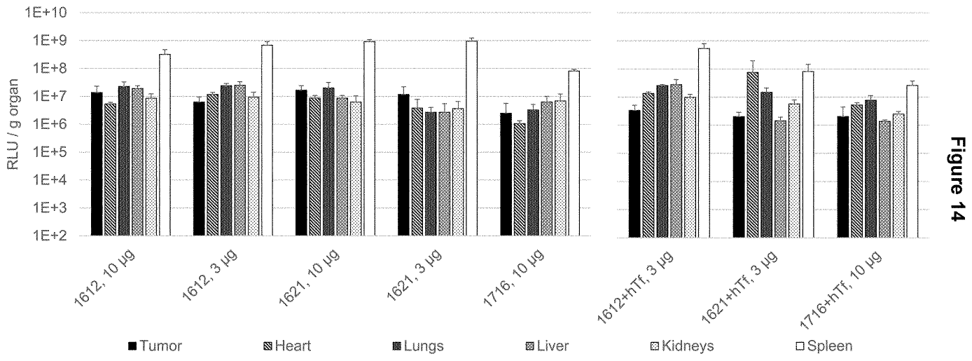

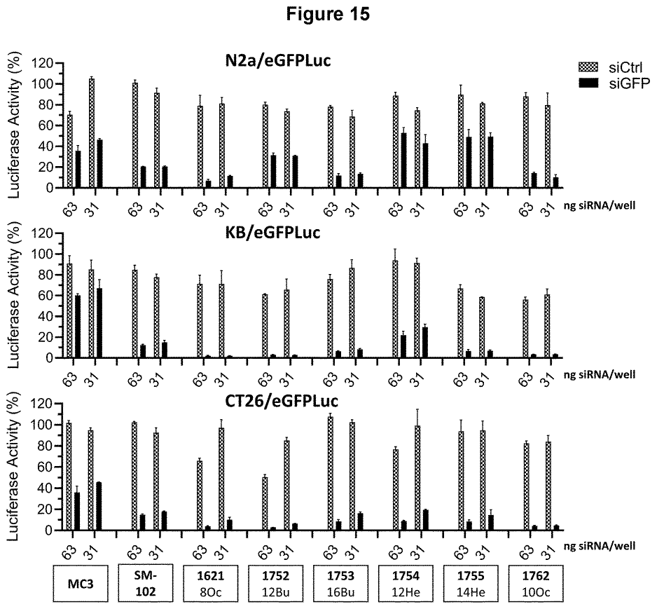

Lipid nanoparticle (LNP) comprising the novel LAF carrier as ionizable compound together with additional lipid components loaded with mRNA were analyzed. mRNA LNPs formulated with the novel LAF carriers resulted in defined, homogeneous nanoparticles with sizes around 150-300 nm and almost neutral zeta potential (Tab. 7). All tested LAF carriers reached higher transfection efficiencies than DLin-MC3-DMA (Patisiran lipid) in N2a cells (FIG. 13A). Especially LAF carrier 1621 (B2, 8Oc, Stp/LAF=1:4) showed a 200-fold higher efficiency than DLin-MC3-DMA and a 4-fold higher efficiency than SM-102 (Moderna lipid). 1621 analogs 1752 (12Bu) and 1755 (14He) as well as U-shape carriers 1716 (U4, 8Oc, Stp/LAF=1:4) and 1720 (U2, 12Oc, Stp/LAF=1:4) showed similar levels of transfection efficiency as SM-102. In HepG2 cells, all carriers were comparable or superior to DLin-MC3-DMA (FIG. 13B). Luciferase expression in Huh7 cells was 2-fold higher for 1755 containing LNPs compared to SM-102 (FIG. 13C). With the exception of 1612, all carriers showed RLU values as high as DLin-MC3-DMA, 1621 and its LAF analogs 1752 (12Bu) and 1754 (12He) even 10-fold higher. Furthermore, mRNA LNPs showed very promising in vivo performance in N2a tumor-bearing mice even at the low dose of 3 μg mRNA/animal, with high luciferase expression levels in all evaluated organs, especially in the spleens but also in lungs, liver, kidneys, heart and tumor (FIG. 14). Targeting with human transferrin led to slightly altered expression patterns and to better biocompatibility.

TABLE 7
Particle size and zeta potential of mRNA LNPs.

| | 1612 | 1613 | 1620 | 1621 | 1716 | 1718 | 1720 | 1752 | 1753 | 1754 | 1755 |
|---|---|---|---|---|---|---|---|---|---|---|---|
| Z- | 275.8 ± | 176.8 ± | 271.1 ± | 288.9 ± | 200.9 ± | 198.1 ± | 209.8 ± | 147.1 ± | 142.1 ± | 171.0 ± | 149.6 ± |
| average | 5.6 | 2.5 | 3.0 | 2.8 | 1.7 | 5.3 | 3.8 | 1.4 | 3.2 | 4.8 | 3.9 |
| in nm | | | | | | | | | | | |
| Pdl | 0.273 ± | 0.159 ± | 0.152 ± | 0.055 ± | 0.150 ± | 0.140 ± | 0.196 ± | 0.204 ± | 0.227 ± | 0.187 ± | 0.220 ± |
| | 0.008 | 0.032 | 0.051 | 0.035 | 0.034 | 0.025 | 0.031 | 0.007 | 0.018 | 0.039 | 0.025 |
| Zeta | 1.88 ± | −3.03 ± | −1.41 ± | −2.96 ± | −0.25 ± | 0.03 ± | 0.70 ± | 3.48 ± | 3.39 ± | 1.11 ± | 2.26 ± |
| potential | 0.11 | 0.42 | 0.21 | 0.11 | 0.13 | 0.19 | 0.17 | 0.12 | 0.60 | 0.81 | 0.33 |
| in mV | | | | | | | | | | | |

Physicochemical characterization regarding size, polydispersity and zeta potential of LNPs containing 12.5 μg/mL CleanCap ® FLuc mRNA (5moU) at N/P 9 and in different molar ratios (see Table A in methods section “mRNA and siRNA lipid nanoparticle (LNP) formulations”).

### Example 6: siRNA LNPs

Lipid nanoparticle (LNP) comprising the novel LAF carrier as ionizable compound together with additional lipid components loaded with siRNA were analyzed. siRNA LNPs formulated with the novel LAF carriers resulted in homogeneous and defined formulations with almost neutral zeta potential (Tab. 8). siRNA LNPs mediated highly promising gene silencing at low siRNA doses (63 and 31 ng/well) without unspecific knockdown and toxicity. The observed variability in efficiency levels of siRNA LNPs in comparison to the positive controls seems to cell line-dependent and may be overcome by further optimizing the conditions. Several structures such as 1621 (B2—8Oc, Stp/LAF=1:4), 1753 (B2—16Bu, Stp/LAF=1:4) and 1762 (B2—10Oc, Stp/LAF=1:4) offer approximately the same potential for eGFP silencing in the three tested cell lines.

TABLE 8
Particle size and zeta potential of siRNA LNPs.

| | 1621 | 1752 | 1753 | 1754 | 1755 | 1762 |
|---|---|---|---|---|---|---|
| Z-average | 350.6 ± | 156.0 ± | 195.1 ± | 173.0 ± | 160.6 ± | 244.3 ± |
| [nm] | 10.7 | 1.0 | 2.0 | 0.6 | 0.2 | 5.9 |
| Pdl | 0.165 ± | 0.192 ± | 0.292 ± | 0.144 ± | 0.287 ± | 0.104 ± |
| | 0.076 | 0.007 | 0.028 | 0.021 | 0.024 | 0.024 |
| Zeta | −2.8 ± | 3.6 ± | 2.6 ± | 2.2 ± | 3.4 ± | −3.2 ± |
| potential | 0.2 | 0.3 | 0.5 | 0.2 | 0.3 | 0.4 |
| [mV] | | | | | | |

Physicochemical characterization regarding size, polydispersity and zeta potential of LNPs containing siCtrl (25 μg/mL) at N/P 9 (see Table A in methods section “mRNA and siRNA lipid nanoparticle (LNP) formulations”).

### Example 7: Cas9 mRNA/sgRNA Polyplexes and Cas9 mRNA/sgRNA/ssDNA Polyplexes

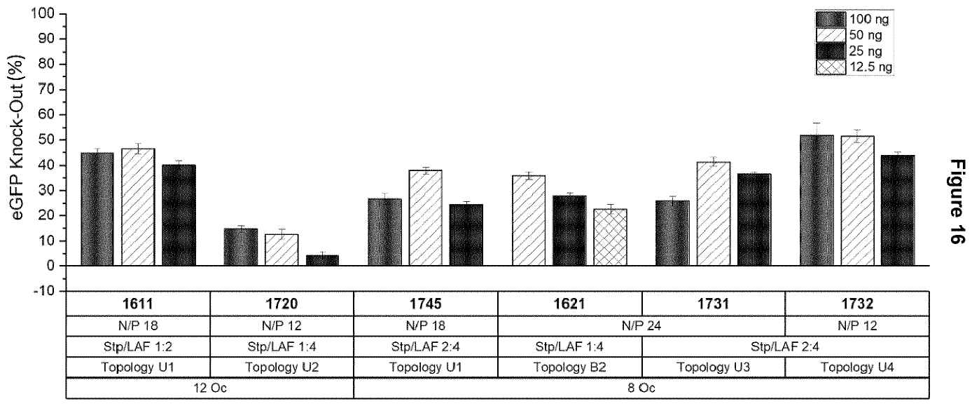

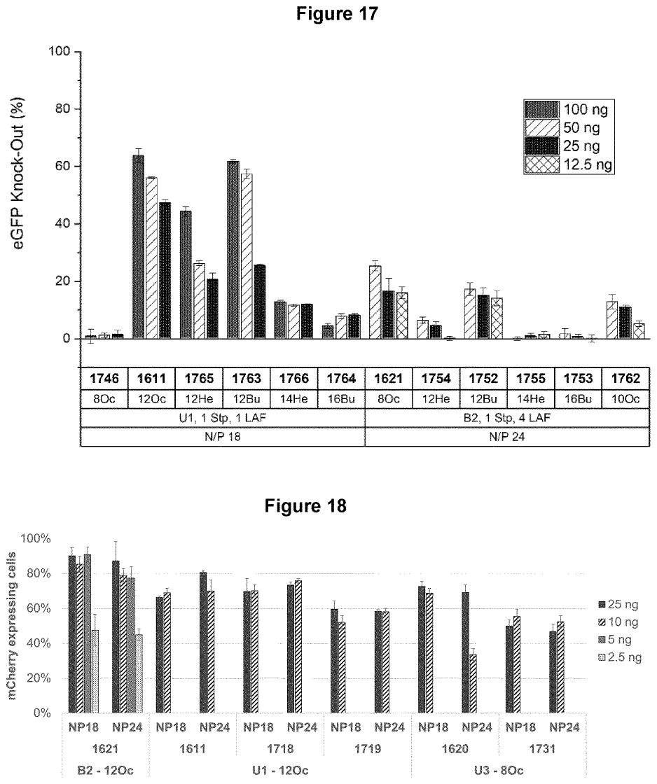

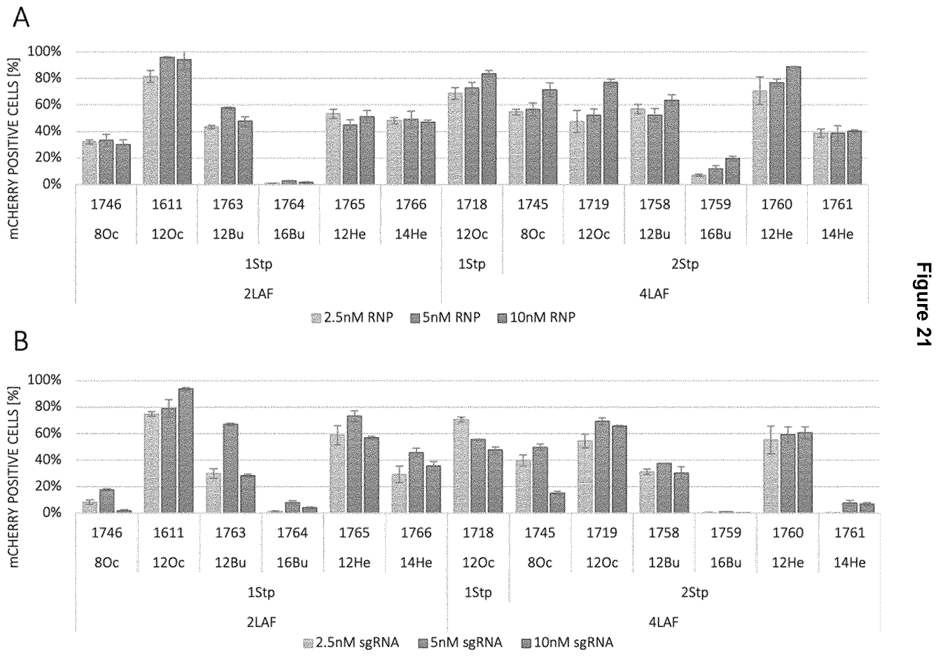

Complexes formed by the novel LAF carriers with Cas9mRNA/sgRNA or Cas9mRNA/sgRNA/ssDNA were characterized and transfections efficiencies of LAF carriers with different topologies were compared and analyzed using the luciferase expression assay. Despite containing nucleic acid cargos of different size (Cas9 mRNA and sgRNA), polyplexes formed with LAF carriers were small, defined nanoparticles (Tab. 9, 10). Even the addition of single stranded donor DNA (ssDNA) did not have a negative influence on the nanoparticle sizes (Tab. 11). Screening of different LAF carriers in N2a/eGFPLuc cells revealed good knock-out efficiency after 72 h for 1611 (U1, 12Oc, Stp/LAF=1:2) and 1732 (U4, 8Oc, Stp/LAF=2:4) (FIG. 16). Again, LAFs with shorter alkyl chains (octyl, decyl, dodecyl) seemed to be more promising than those with longer ones (tetradecyl, hexadecyl) (FIG. 17). Furthermore, LAF carriers promoted high gene editing in HeLa mCherry-DMDex23-eGFP cells at very low total RNA doses (FIG. 18). B2 carrier 1621 (12Oc, Stp/LAF 1:4) mediated gene editing even at very low doses of 5 and 2.5 ng of total RNA per well. The high gene editing efficiency at very low total RNA doses remained even after incubation in full serum (FIG. 19), indicating persisting polyplex integrity and protection of the nucleic acid cargo from serum nucleases. For HDR experiments (FIG. 20), 1611 was chosen due to high mRNA/pDNA co-delivery efficiency (FIG. 9). Very high editing percentage of cells but also high HDR-mediated conversion could be achieved with 1611 polyplexes. A w/w ratio of total RNA to ssDNA of 1:0.5 was figured out to be most suitable (FIG. 20B).

TABLE 9
Particle size and zeta potential Cas9 mRNA/sgRNA polyplexes.

| | 1611 | 1745 |
|---|---|---|
| | (N/P 18) | N/P 18) |
| Z-average in nm | 98.1 ± 1.8 | 99.4 ± 7.9 |
| Pdl | 0.235 ± 0.011 | 0.266 ± 0.046 |
| Zeta potential in mV | 14.1 ± 2.5 | 11.9 ± 1.6 |

Comparison of different LAF carriers. Cas9 mRNA/sgGFP1 polyplexes formed at indicated N/P ratios and a combined nucleic acid concentration of 5.0 μg/mL. Data presented as average of three sub-runs ± SD.

TABLE 10
Particle size and zeta potential Cas9 mRNA/sgRNA polyplexes.

| | 1621 | 1611 | 1718 | 1719 | 1620 | 1731 |
|---|---|---|---|---|---|---|
| Z-average | 226.7 ± | 88.2 ± | 107.5 ± | 80.8 ± | 134.3 ± | 67.5 ± |
| in nm | 5.1 | 4.8 | 3.1 | 1.4 | 0.2 | 2.3 |
| Pdl | 0.570 ± | 0.219 ± | 0.223 ± | 0.268 ± | 0.130 ± | 0.201 ± |
| | 0.147 | 0.029 | 0.015 | 0.022 | 0.034 | 0.027 |
| Zeta potential | 20.8 ± | 36.6 ± | 36.0 ± | 42.6 ± | 22.7 ± | 37.5 ± |
| in mV | 0.5 | 0.9 | 2.4 | 0.9 | 2.1 | 3.8 |

Comparison of different LAF carriers.
Cas9 mRNA/sgDMDex23 polyplexes formed at N/P 24 and a combined nucleic acid concentration of 12.5 μg/mL.
Data presented as average of three sub-runs ± SD.

TABLE 11
Particle size and zeta potential
Cas9 mRNA/sgRNA/ssDNA polyplexes.

| RNA:ssDNA | 1:0 | 1:0.5 | 1:1 |
|---|---|---|---|
| Z-average in nm | 84.3 ± 1.5 | 86.5 ± 0.1 | 88.5 ± 1.2 |
| Pdl | 0.234 ± 0.009 | 0.288 ± 0.017 | 0.348 ± 0.013 |
| Zeta potential in mV | 30.2 ± 1.3 | 26.3 ± 1.9 | 25.7 ± 1.0 |

Comparison of different weight ratios of RNA (CleanCap ® Cas9 mRNA (5 moU) and sgGFP) to ssDNA template. Co-formulation of Cas9 mRNA/sgRNA and ssDNA with carrier 1611 into polyplexes at N/P 18 and a combined nucleic acid concentration of 5 μg/mL. Data presented as average of three sub-runs ± SD.

### Example 8: Cas9 mRNA/sgRNA Polyplexes and Cas9 mRNA/sgRNA/ssDNA Polyplexes

CRISPR Cas9 gene editing lipoplexes containing either Cas9 protein/sgRNA ribonucleoprotein (RNP) (FIG. 21A) or Cas9 mRNA and sgRNA (FIG. 21B) were tested on HeLa mCherry-DMDex23-eGFP. High gene editing efficiency with similar tendencies for different LAF carriers were demonstrated for both delivery platforms. The LAF 1611 exhibits the highest efficiency for both RNP and Cas9mRNA/sgRNA lipopolyplexes.

### Example 9: Carriers Replacing Stp by Different Polar Building Blocks (Stp Analogs)

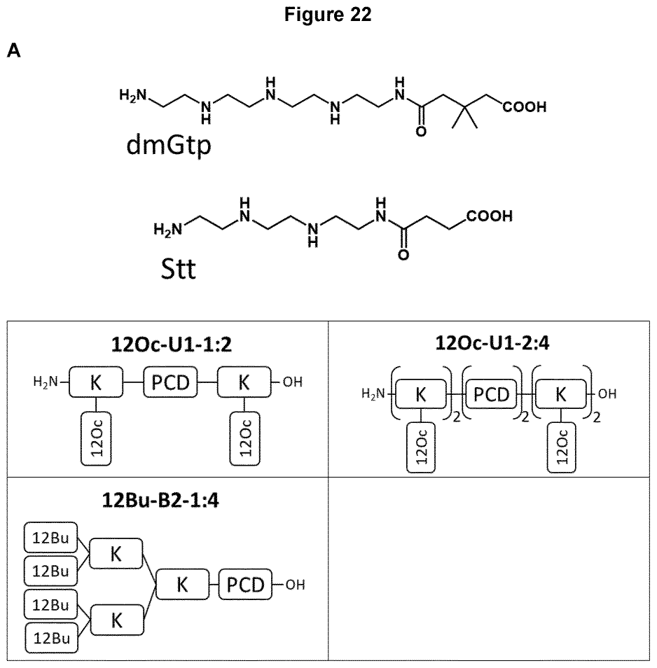

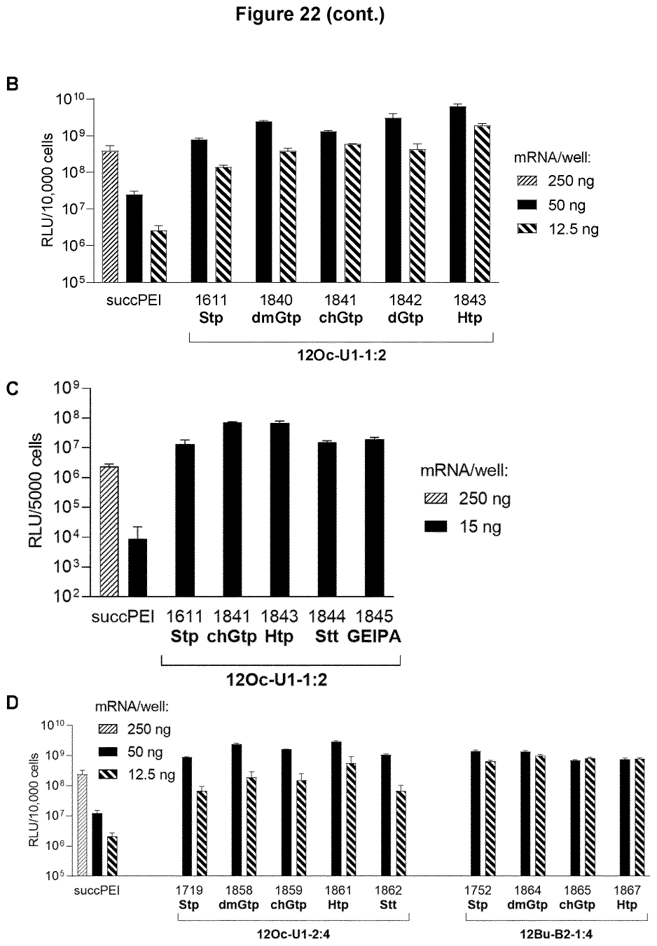

LAF carriers of different topologies containing various polar cationizable domains replacing Stp were synthesized (FIG. 22A, Tab. 2). Polyplexes were formed as described in 6.1 with dilution of the carrier in purified water instead of HBG. Transfection efficiency was tested as described in 6.9 with transfection of either 20, 4, 1.2 or 1 μL of polyplex solution (12.5 μg/mL mRNA). Before treatment, cell culture medium was replaced by suitable volumes of fresh medium containing 10% (v/v) FBS to reach a final volume of 100 μL per well aftertreatment. The effectiveness of the different polar cationizable domains for mRNA delivery was demonstrated in a luciferase assay in N2a cells for different topologies (i.e., 12Oc-U1-1:2, 12Oc-U1-2:4 and 12Bu-B2-1:4) (FIGS. 22B and D). Corresponding mRNA polyplexes outperformed the positive control succPEI even at low doses and reached comparable or even higher luciferase expression levels than their Stp analogs (FIG. 22B-D). Carriers of the topology 12Oc-U1-1:2 also displayed high efficiency in DC2.4 dendritic cells (FIG. 22C). These results show the possibility of tuning and improving the LAF carriers by variations of the polar cationizable domain which can be beneficial for different applications.

### Example 10: Novel Carriers Connecting Stp and LAF Via Ornithine as Branching Connector

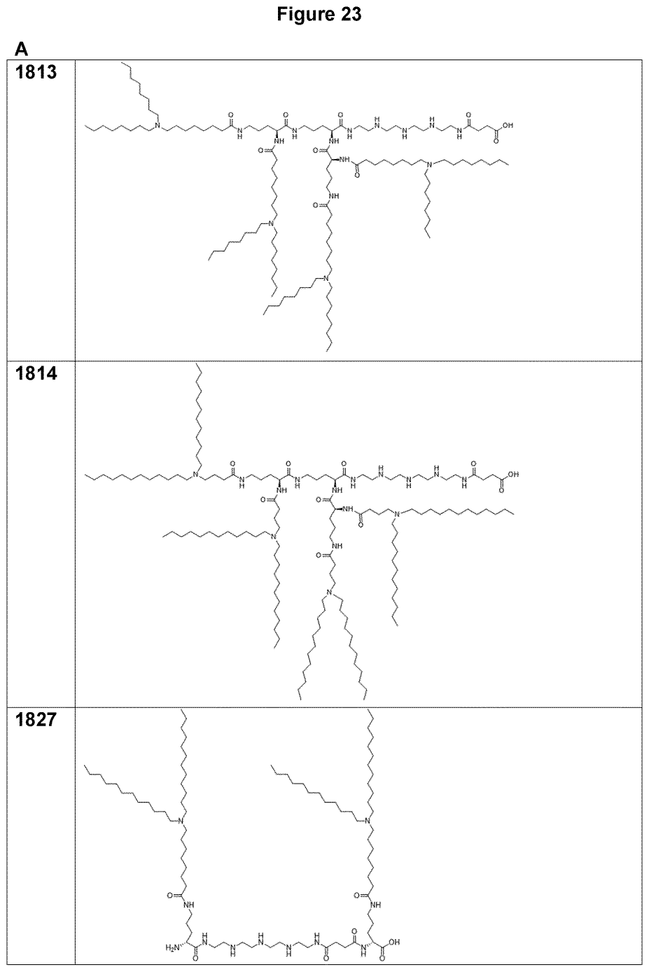

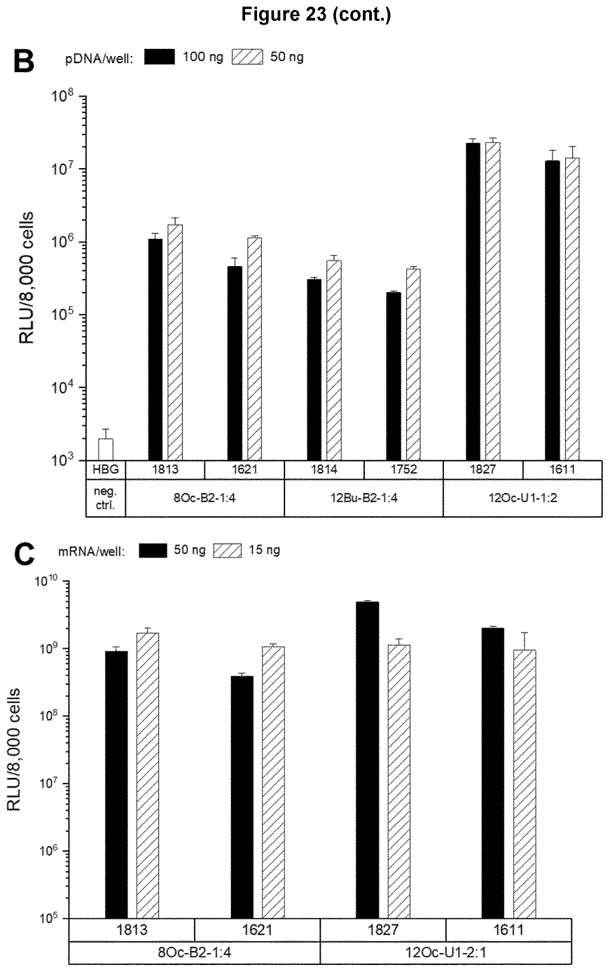

The novel carriers were synthesized by connecting the polar (Stp) and apolar (LAF) domain with ornithine instead of lysine (FIG. 23A, Tab. 3.1). The introduction of ornithine lead to shorter interspaces within the two domains. Polyplexes were formed as described in 6.1, diluting the carriers in purified water instead of HBG. Transfection efficacy was evaluated by adaption of the protocol described in 6.9. For pDNA either 5 or 1.5 μL of polyplex solution (10 μg/mL pDNA) whereas for mRNA 4 or 1.2 μL of polyplex solution (12.5 μg/mL mRNA) were transfected. Before treatment, cell culture medium was replaced by fresh medium containing 10% (v/v) FBS to reach a final volume of 100 μL per well after treatment. In terms of transfection efficacy, the novel carriers outperformed corresponding lysine analogs for both pDNA (FIG. 23B) and mRNA (mRNA, FIG. 23C), especially at the mRNA dose of 50 ng/well. In case of B2 carriers, the lower dose of 15 ng/well lead to even higher RLU values than 50 ng/well. In direct comparison, carriers benefit from replacing lysine with ornithine.

### Example 11: Bioreducible LAF Carriers Containing Disulfide Building Blocks

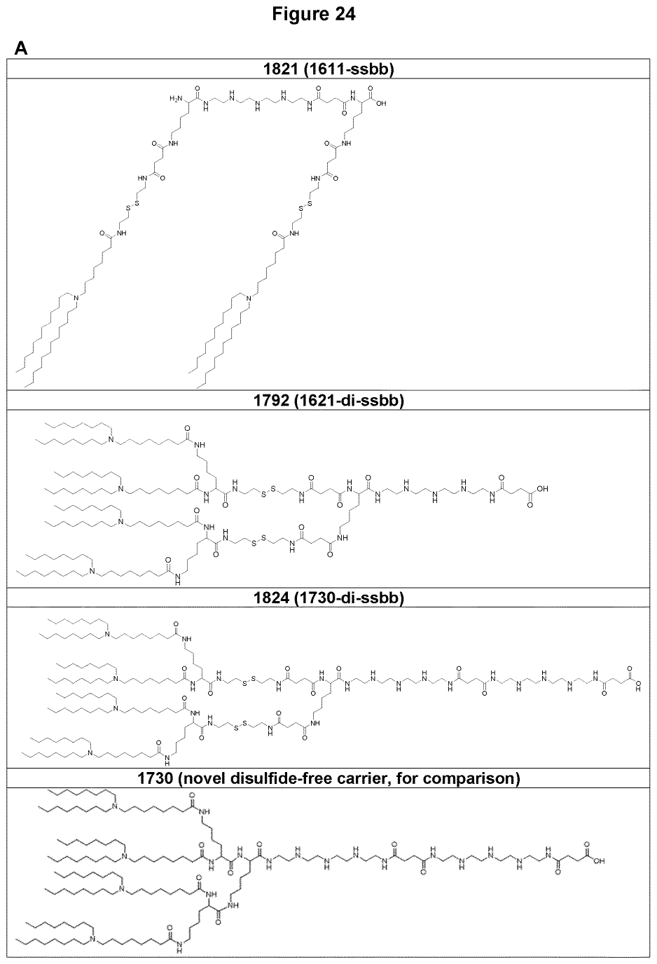

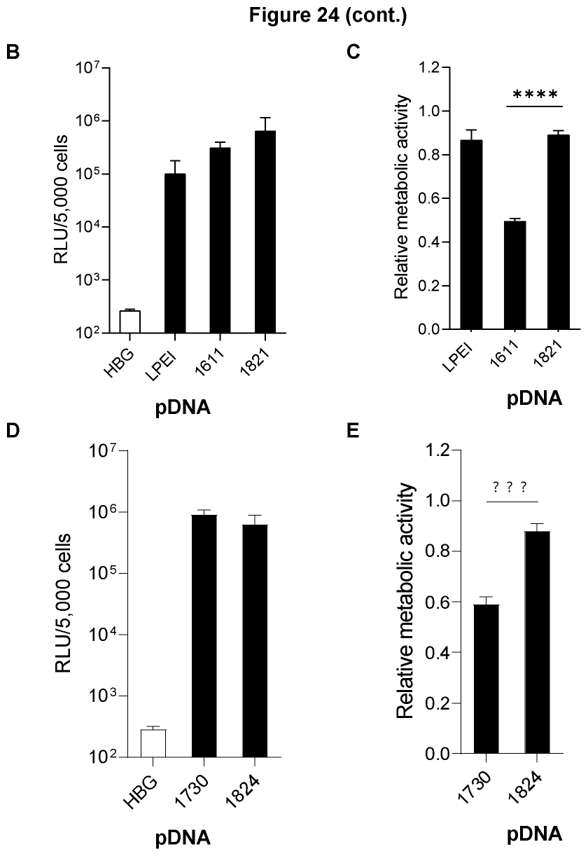

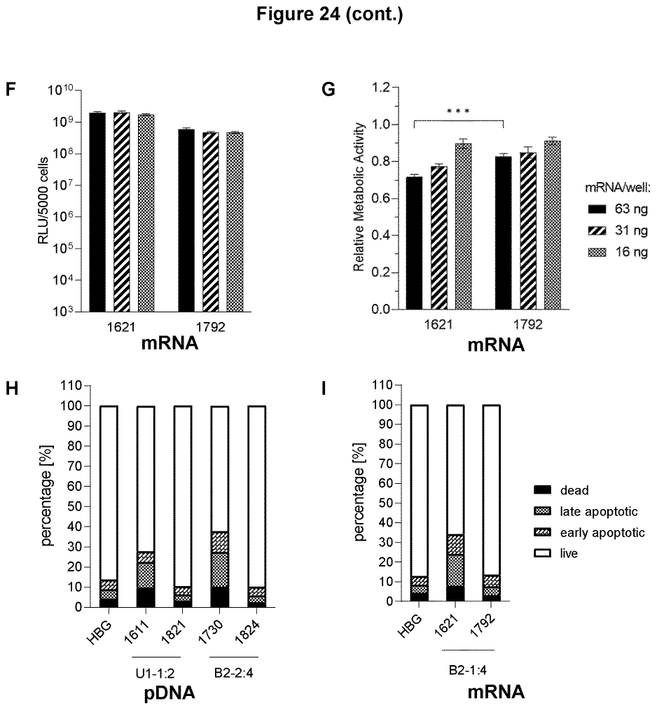

Carriers containing bioreducible elements were synthesized with a precise positioning of the disulfide building blocks in the sequences to allow reductive cleavage into mostly positively charged Stp-fragments and lipophilic LAF-domains (FIG. 24A, Tab. 3.2). The disulfide-containing carriers were able to form defined homogenous polyplexes with pDNA and mRNA with positive zeta potential (Tab. 12). Polyplexes were formed as described in 6.1 with dilution of the carrier in purified water instead of HBG. Transfection efficiency was tested as described in 6.9 with transfection of 20 μL of pDNA polyplex solution (10 μg/mL pDNA) or either 5, 2.5 or 1.25 μL of polyplex solution (12.5 μg/mL mRNA). Before treatment, cell culture medium was replaced by fresh medium containing 10% (v/v) FBS to reach a final volume of 100 μL per well after treatment. They outperformed positive control LPEI in terms of transfection efficiency and reached comparable high luciferase expression levels than their non-reducible analogs, both for pDNA (carriers 1821 and 1824, FIGS. 24B and D) and mRNA (carrier 1792, FIG. 24F) but were significantly less toxic, as determined via the CellTiter Glo® assay (pDNA, FIG. 24C, E; mRNA, FIG. 24G) as described in 6.16. For example, at 25% of the standard mRNA dose of 250 ng/well, the disulfide-containing structure 1792 showed significantly improved cell viability compared to the non-reducible carrier 1621 (FIG. 24G). The benefit of the bioreducible LAF analogs regarding improved biocompatibility was further confirmed in an annexin/propidium iodide (PI) assay (FIGS. 24H and I) as described in 6.17. In direct comparison of LAF carriers with or without the disulfide motif within one topology, complexed either with pDNA or mRNA, the amount of late or early apoptotic (i.e., annexin-positive) cells as well as dead cells was greatly reduced for the bioreducible analogs of each set.

TABLE 12
Particle size and zetapotential of pDNA and mRNA polyplexes
formed with disulfide containing LAF carriers.

| | pDNA (N/P 18) | | | mRNA (N/P 24) |
|---|---|---|---|---|
| | 1730 | 1824 | 1821 | 1792 |
| Z-average [nm] | 95.3 ± 1.6 | 93.7 ± 2.6 | 122.9 ± 1.7 | 117.0 ± 1.1 |
| PDI | 0.10 ± 0.01 | 0.10 ± 0.03 | 0.08 ± 0.03 | 0.11 ± 0.01 |
| Zetapotential [mV] | 21.7 ± 0.6 | 20.2 ± 0.4 | 21.4 ± 2.4 | 14.7 ± 0.3 |

Comparison of different LAF containing carriers formulated as mRNA or pDNA polyplexes at indicated N/P ratios and at a concentration of 10 μg/mL of pCMVLuc or 12.5 μg/mL of CleanCap ® FLuc mRNA (5 moU), respectively. Data presented as average of three sub-runs ± SD.

### Example 12: Novel Carrier Containing εK as Polar Cationizable Domain (PCD) Instead of Stp

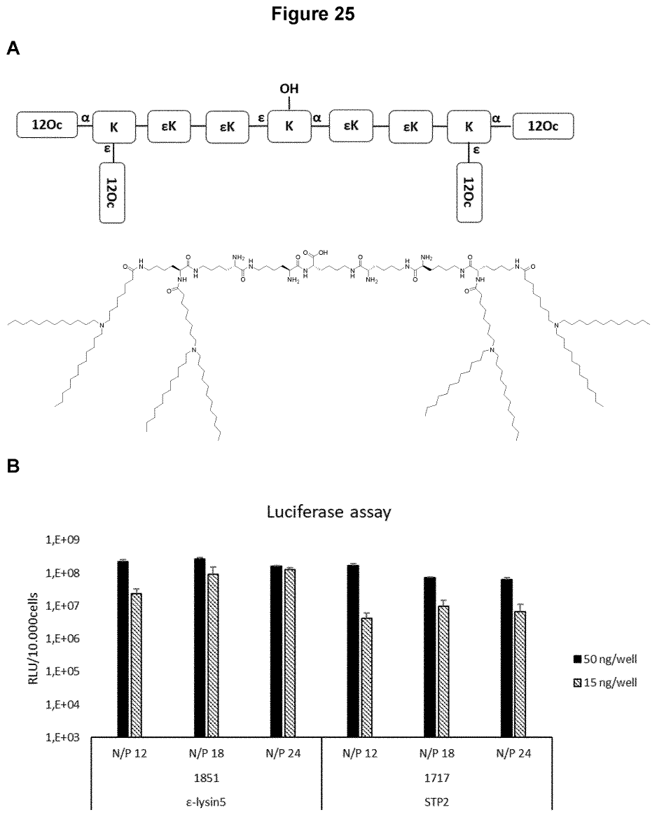

The novel εK containing carrier 1851 was synthesized to create a symmetric structure with two identical arms. As polar cationizable domain (comprising two εK2 motives linked via a lysine) was introduced to mimic Stp2 (FIG. 25A, Tab. 3.3). Polyplexes were formed as described in 6.1, diluting the carriers in purified water instead of HBG. Transfection efficacy was evaluated by adaption of the protocol described in 6.9. For mRNA polyplexes, 4 or 1.2 μL of polyplex solution (12.5 μg/ml mRNA) were transfected and suitable amounts of purified water/HBG mixture (1:1) were added to reach a final volume of 100 μL per well after treatment. The εK containing polyplexes outperformed its Stp2 analog (carrier 1717; 12Oc U4 2:4) in terms of transfection efficiency at all tested N/P ratios and mRNA doses per well. At the lower tested mRNA dose of 15 ng/well, εK carriers reached around 10-fold higher RLU values than their Stp2 analog.

### Example 13. Carriers Applied for Delivery of Cytotoxic Poly(Inosine:Cytosine), Poly(I:C)

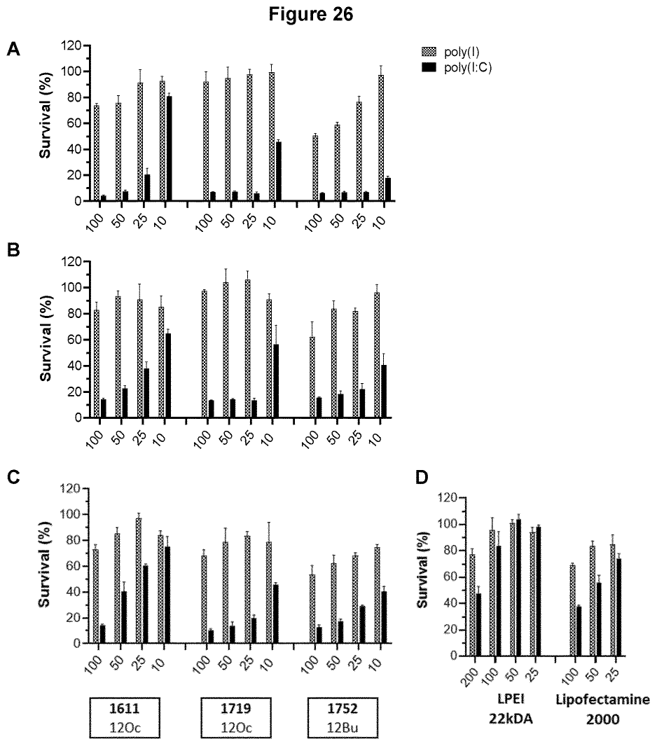

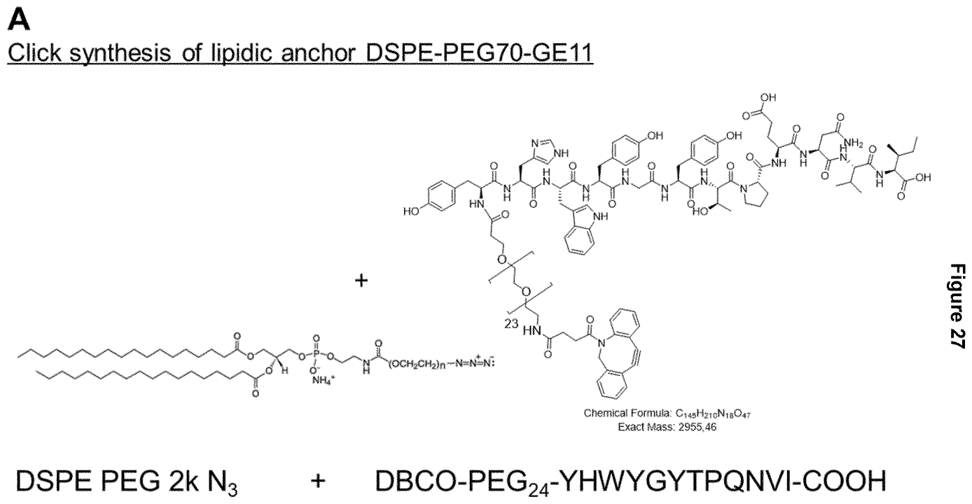

The cytotoxic effect of double-stranded RNA (poly(I:C)) was evaluated in cancer cells using LAF carriers 1611, 1719, and 1752. The toxicity induced by poly(I:C) and carriers were differentiated by using non-toxic single-stranded poly(I). LPEI and the LAF-Stp carriers were transfected in medium supplemented with 10% (v/v) FBS. In the case of lipofectamine, particles were prepared and transfected according to the manufacturer's protocol (particle formation as well as transfection in serum-free medium; ThermoFisher). At 4 h after lipofectamine transfection, medium was replaced with fresh medium supplemented with 10% (v/v) FBS followed by incubation of 48 h in total. The percentage of cell survival was calculated relative to the HBG buffer-treated control cells (n=3, mean+SD). The well-formed, homogenous polyplexes (Tab. 13) could efficiently deliver poly(I:C) into the target cells, leading to tumor cell death in a dose- and cell line-dependent manner (FIGS. 26A, B and C). At the similar poly(I:C) doses of 100, 50, and 25 ng per well, all LAF polyplexes outperformed the positive controls LPEI and lipofectamine (representative positive controls in KB cells are shown in FIG. 26D). Interestingly, a significant decrease in the cell viability was induced by LAF carriers 1719 and 1752 at lower concentration of 10 ng poly(I:C) per well, especially in KB cells. No significant toxicity was observed by control poly(I) polyplexes at low effective doses.

TABLE 13
Particle size and zetapotential of poly(I:C) and poly(I) polyplexes
formed with LAF carriers of different topologies.

| | | 1611 (N/P 18) | 1719 (N/P 12) | 1752 (N/P 24) |
|---|---|---|---|---|
| Z-average in nm | poly(I) | 125.9 ± 1.8 | 86.8 ± 11.4 | 108.6 ± 2.4 |
| | poly(I:C) | 58.3 ± 0.8 | 95.9 ± 2.1 | 72.3 ± 2.1 |
| Pdl | poly(I) | 0.32 ± 0.04 | 0.32 ± 0.08 | 0.18 ± 0.05 |
| | poly(I:C) | 0.27 ± 0.02 | 0.25 ± 0.05 | 0.27 ± 0.03 |
| Zetapotential in mV | poly(I) | 16.7 ± 1.8 | 23.2 ± 0.4 | 23.3 ± 0.5 |
| | poly(I:C) | 20.5 ± 3.4 | 13.8 ± 2.9 | 23.9 ± 3.0 |

Comparison of different LAF containing carriers formulated as poly(I:C) and poly(I) polyplexes at indicated N/P ratios and at a concentration of 12.5 μg/mL of poly(I:C) and poly(I), respectively. Data presented as average of three sub-runs ± SD.

### Example 14. EGF Receptor-Targeted PEG-Shielded pDNA Polyplexes

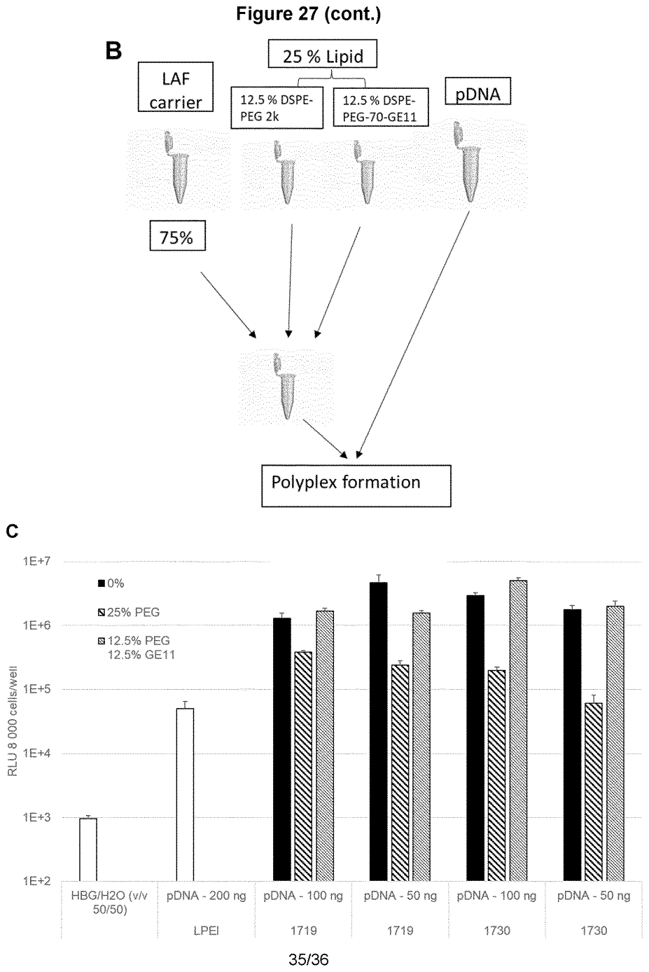

Unmodified polyplexes of carriers 1719 and 1730 (Tab. C) were capable of forming homogeneous defined particles with positive zeta potential (Tab. 14). Synthesis of the lipidic anchor DSPE-PEG70-GE11 comprising the targeting ligand GE11 (YHWYGYTPQNVI; SEQ ID NO: 9) from DSPE PEG 2K N3 and DBDO-PEG24-GE11 via click chemistry as well as formulated of polyplexes comprising said ligand are described in 6.19 and as illustrated in FIGS. 27A and B. Transfection efficiency was evaluated by adaption of the protocol described in 6.9. For pDNA polyplexes, 10 or 5 μL of polyplex solution (10 μg/mL pDNA) were transfected. Before treatment, cell culture medium was replaced by fresh medium containing 10% (v/v) FBS to reach a final volume of 100 μL per well upon treatment. The incorporation of 25% DSPE-PEG2k-N3 or the incorporation of DSPE-PEG70-GE11 conjugate as EGF receptor targeting lipid did not lead to a notable change in size or Pdl, but the zeta potential was notably reduced (Tab. 14). Furthermore, the three different formulations were tested regarding their transfection efficiency in Huh7 cells (FIG. 27). As expected, shielding led to drastically reduced transfection levels (RLU) compared to unmodified polyplexes. However, transfection efficiency could be maintained or even slightly improved by introducing the targeting ligand GE11 via the DSPE-PEG70-GE11 conjugate. In comparison to shielded polyplexes, targeted 1719 polyplexes reached 4.4-fold (pDNA dose 100 ng/well) and 6.5-fold (pDNA dose 50 ng/well) higher RLU values. In the case of 1730, this was even more prominent, where a 25.5-fold increase (pDNA dose 100 ng/well) and a 32.8 increase (pDNA dose 50 ng/well) was seen.

TABLE 14
Particle size and zeta potential of shielded and targeted
pDNA polyplexes in comparison to unmodified polyplexes.

| | | Unmodified | PEGylated | Targeted Polyplexes |
|---|---|---|---|---|
| | | Polyplexes | Polyplexes | 12.5% PEG 2k, |
| | | 0% | 25% PEG | 12.5% PEG-GE11 |
| 1719 | Size | 77 | 76 | 88 |
| | Pdl | 0.09 | 0.13 | 0.13 |
| | Zeta | 29.5 | 10.5 | 10.5 |
| 1730 | Size | 109 | 100 | 125 |
| | Pdl | 0.21 | 0.22 | 0.17 |
| | Zeta | 15.9 | 2.0 | 2.0 |

Different LAF containing carriers formulated as pDNA polyplexes at indicated N/P ratios and at a concentration of 10 μg/mL of pCMVLuc.

### Example 15. In Vivo Expression of Luciferase mRNA Polyplexes or LNPs after Intramuscular Injection

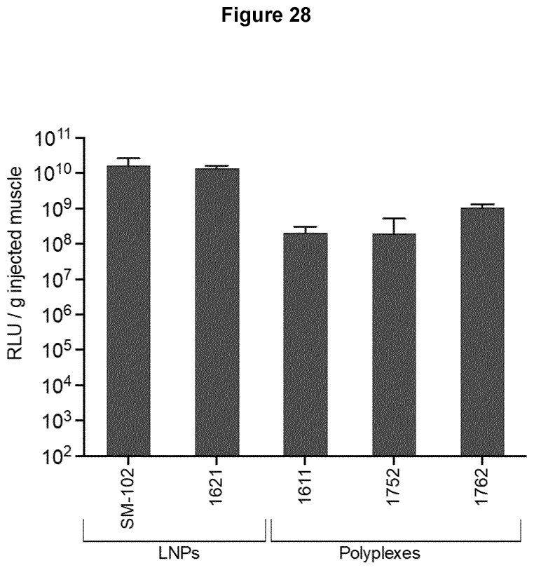

Intramuscular application of 3 μg CleanCap FLuc (5moU) mRNA polyplexes or LNPs in 50 μL HBG into the musculus biceps femoris of 6 week old BALB/c mice (n=4; mean+SD). Luciferase expression was evaluated at 6 h post treatment via an ex vivo luciferase assay of the injected muscle. Polyplexes of LAF carriers 1611, 1752, and 1762 were all formed at an N/P ratio of 24. For LNPs, the N/P ratio of LAF carrier 1621 and SM-102 was 24 and 6, respectively. Molar ratios of lipidic components in applied LNP formulations were 38.5:10:1.5:50 mol % (Chol:DSPC:PEG-DMG:SM-102) and 47.6:23.8:4.8:23.8 mol % (Chol:DOPE:PEG-DMG:1621) for SM-102 and 1621 LNPs, respectively. mRNA polyplexes and LNPs formulated with LAF carriers mediated very high luciferase expression levels in the injected muscle at 6 h after intramuscular injection (FIG. 28), comparable to the positive control SM-102 LNP.

1. A carrier comprising at least one polar cationizable domain (PCD), two or more apolar cationizable domains (ACD) and at least one branching connector (BC), wherein the two or more ACDs are linked by at least one branching connector to at least one PCD,
wherein
(a) the PCD
(i) is an oligo(alkylamino) acid of formula I: 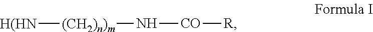
wherein n=2 or 3, alternating 2 and 3 or alternating 3 and 2; and m=3 or 4; and
wherein R is
—(CH₂)_{y}—CO₂H, wherein y=2, 3 or 4; 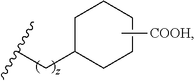
wherein z=0 or 1;
—CH₂-(cyclohexylene)-CH₂—CO₂H;
—CH₂—C(CH₃)₂—CH₂—CO₂H or CH₂—C(CH₂—CH₃)₂—CH₂—CO₂H;
—CH₂—O—CH₂—CO₂H;
—CH₂—NH—CH₂—CO₂H; or
(ii) is an epsilon-poly-L-lysine comprising structure, preferably (ε-lysine)ₙ-6-amino hexanoic acid ((^{ε}K)ₙ-6-Ahx), (ε-lysine)ₙ₊₁ or (ε-lysine)ₙ, wherein n=2, 3 or 4, preferably 2 or 3;
(b) the BC is a bisamide connector selected from the group consisting of L-lysine, L-ornithine and an artificial amino acid comprising two amino groups and a carboxyl group; and
(c) the ACD is a lipo amino fatty acid (LAF) comprising a tertiary amine of formula II: 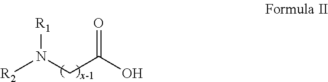
wherein x is 4-12, and R1 and R2 is an acyclic alkane or alkene, wherein R1 is C6-C16 and R2 is C1-C16 and wherein R1 and R2 may be the same or different.

2. The carrier of claim 1, wherein the oligo(alkylamino) acid is a tetraethylenepentamine or a triethylenetetramine of formula Ib:

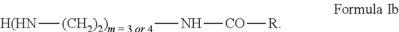

3. The carrier of claim 1, wherein the carrier further comprises (a) a disulfide building block between the at least one PCD and the two or more ACDs, and/or (b) a spacer between the at least one PCD and the two or more ACDs.

4. The carrier of claim 1, wherein the carrier further comprises a terminal functional group selected from an azido-group and a thiol group, preferably wherein the carrier comprises a terminal azido-lysine, azido-hexane, cysteine or homocysteine and/or a targeting ligand coupled via the azido-group or the thiol group.

5. The carrier of claim 1, wherein the carrier may further comprise one or more histidine, cysteine and/or arginine.

6. The carrier of claim 1, wherein the carrier has a topology selected from the group consisting of:
(a) a bundle structure 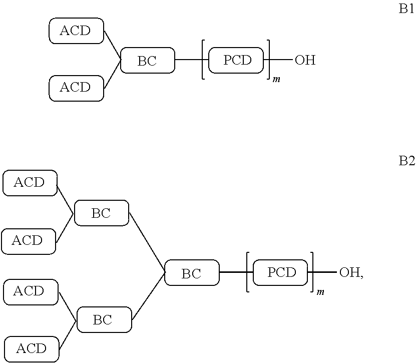
wherein m=1, 2 or 3; and
(b) a U-shape structure 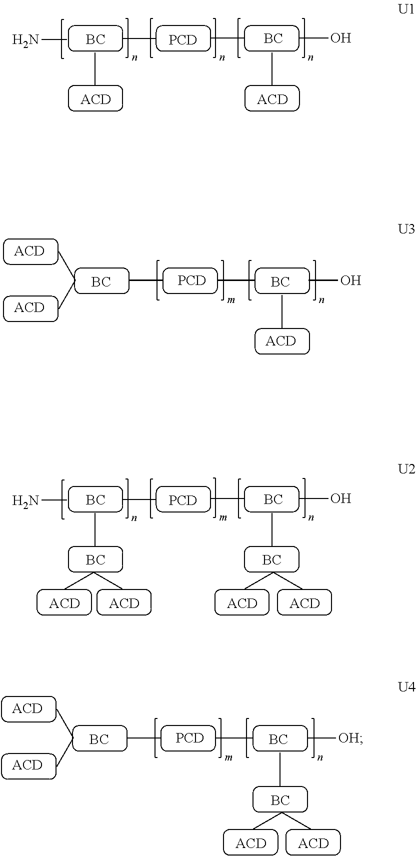
wherein n=1 or 2 and m=1, 2 or 3, preferably 1 or 2.

7. The carrier of claim 1, wherein the PCD to ACD ratio is between 1:2 to 1:4, preferably 1:2 for U-shapes and/or 1:2 to 1:4 for B2 structures, more preferably 1:2 for U-shapes comprising one or two PCDs, 1:4 for B2 structures comprising one PCD or 1:2 for B2 structures comprising two PCDs.

8. The carrier of claim 1, for nucleic acid and/or protein delivery, preferably for nucleic acid delivery.

9. A nanoparticle comprising the carrier of claim 1, further comprising a cargo, wherein the cargo comprises a nucleic acid and/or a protein, preferably the cargo comprises RNA or DNA, preferably the cargo comprises mRNA, Cas mRNA/gRNA, siRNA, miRNA, polyinosinic:polycytodylic acid (poly(I:C)), phosphodiamidate-morpholino-oligomer (PMO), a non-viral DNA expression vector, Cas protein/gRNA ribonucleoprotein (RNP) or mixtures thereof.

10. The nanoparticle of claim 9, wherein the nanoparticle is a complex (polyplex) formed by the carrier mixed with the cargo, a lipid nanoparticle (LNP) comprising the carrier loaded with the cargo, or a complex formed by the cargo covalently coupled to the carrier.

11. A pharmaceutical composition comprising the nanoparticle of claim 9 further comprising at least one pharmaceutically acceptable excipient.

12. A method of therapy comprising administering the nanoparticle of claim 9 to a subject.

13. A method of treating or preventing cancer, a genetic disease, an infectious disease or an autoimmune disease, the method comprising administering to a subject in need thereof, the nanoparticle of claim 9.

14. An in vitro method for introducing a nucleic acid and/or a protein into mammalian cells comprising contacting a mammalian cell in vitro with the nanoparticle according to claim 9 comprising a nucleic acid and/or a protein as cargo, preferably mRNA, Cas mRNA/gRNA, siRNA, miRNA, polyinosinic:polycytodylic acid (poly(I:C), phosphodiamidate-morpholino-oligomer (PMO), a non-viral DNA expression vector, or Cas protein/gRNA ribonucleoprotein (RNP).

15. A method of cellular delivery comprising use of the carrier of claim 1 for cellular delivery of a nucleic acid and/or a protein, preferably of a nucleic acid.

16. A method of cellular delivery comprising use of the nanoparticle of claim 9 for cellular delivery of a nucleic acid and/or a protein, preferably of a nucleic acid.
